(19) United States
(12) Patent Application Publication
(10) Pub. No.: US 2026/0224459 A1
(43) Pub. Date: Aug. 6, 2026

(54) PROCESS FOR TREATING KERATIN FIBRES USING A COMPOUND DERIVED FROM THE CONDENSATION OF POLY(THI)OL AND OF ACETOACETATE AND A CROSSLINKING AGENT

(71) Applicant: L'OREAL, Paris (FR)

(72) Inventors: Boris STEVANT, Aulnay-sous-Bois (FR); Laurent SABATIE, Aulnay-sous-Bois (FR); Etienne SASONNET, Aulnay-sous-Bois (FR)

(73) Assignee: L'OREAL, Paris (FR)

(21) Appl. No.: 19/135,641

(22) PCT Filed: Dec. 12, 2023
(86) PCT No.: PCT/EP2023/085432

§ 371 Date: Jun. 4, 2025

(30) Foreign Application Priority Data
Dec. 13, 2022 (FR) FR2213265

(51) Int. Cl. A61K 8/37; A61K 8/19; A61K 8/34; A61K 8/41; A61K 8/58; A61K 8/892; A61K 8/898; A61K 8/899; A61Q 5/06

(52) U.S. Cl. CPC A61K 8/375; A61K 8/19; A61K 8/34; A61K 8/41; A61K 8/585; A61K 8/898; A61K 8/899; A61Q 5/065; A61K 8/892; A61K 2800/43; A61K 2800/884; A61K 2800/95

(57) ABSTRACT

The present invention relates more particularly to the cosmetic field of keratin fibres, and notably to that of caring for, styling and/or dyeing keratin fibres and preferably the hair. The invention is thus directed towards proposing a process using on keratin fibres in one or more steps i) at least one compound of formula (I) derived from the condensation of poly(thi)ol or polyamine and of acetoacetate (ACAC) and ii) at least one crosslinking agent. The invention also relates to a composition comprising ingredients i) and ii), and also to a kit comprising i) and ii).

### RELATED APPLICATIONS

The present application is a national stage application that claims priority to International Application No. PCT/EP2023/085432 filed Dec. 12, 2023, which claims priority to French Application No. 2213265 filed Dec. 13, 2022, the disclosures of which are incorporated herein by reference in their entirety.

### TECHNICAL FIELD OF THE INVENTION

The present invention relates more particularly to the cosmetic field of keratin fibres, and notably to that of caring for, styling and/or dyeing keratin fibres and preferably the hair. The invention is thus directed towards proposing a process using on keratin fibres in one or more steps i) at least one compound of formula (I) derived from the condensation of poly(thi)ol and acetoacetate (ACAC) and ii) at least one crosslinking agent. The invention also relates to a composition comprising ingredients i) and ii), and also to a kit comprising i) and ii).

### BACKGROUND OF THE INVENTION

Cosmetic products conventionally require the use of one or more film-forming polymers in order to obtain a quality deposit of these products on keratin fibres, and in particular to satisfy the expectations detailed below.

Moreover, in the field of haircare, a new range of products known as “Hair Makeup” has recently been developed. These products guarantee temporary hair dyeing that lasts after 1 to 3 shampoo washes. They are thus a particularly attractive alternative for consumers to permanent hair dyeing, provided, of course, that the colouring effect is effectively guaranteed to last after contact with water and a few shampoo washes. This requirement is also notably satisfied by the use of effective film-forming agents. Thus, FR 2 741 530, for example, proposes for this purpose, for the temporary dyeing of keratin fibres, the use of a dispersion of film-forming polymer particles including at least one acidic function and at least one pigment dispersed in the continuous phase of said dispersion. The colourings obtained via this dyeing method are not always satisfactory in terms of persistence, notably with respect to shampoo washing. Specifically, in general, these polymers do not afford deposits on keratin fibres that satisfy all the abovementioned requirements, namely very good water resistance, in particular with respect to shampoo washing for the hair, which make it possible to adjust the gloss or invisibility qualities and which, in the case of haircare use, provide very satisfactory styling hold.

US 2006/0079599 describes a polymer-based tissue adhesive for medical use, including certain polymers bearing ACAC groups, which are combined with polyamines in the form of a hydrogel. EP 3 250 177 A1 describes the use of acetoacetylated compounds in the field of keratin fibres. Keratin fibres treated with said acetoacetylated compounds do not always give satisfactory results, notably in terms of persistence with respect to water and to shampoo washing.

There is also a need for cosmetic dye compositions intended for application to the hair which have good resistance to water and to shampoo washing in order to ensure colour persistence over time notably in terms of intensity and/or chromaticity.

There is also a need for cosmetic compositions, notably non-colouring compositions, intended for hair application, which have styling properties, notably hair hold, particularly curl hold, and which are resistant with respect to water and to shampoo washing.

The present invention is precisely directed towards meeting all or some of these needs.

### SUMMARY OF THE INVENTION AND DETAILED DESCRIPTION

These problems are solved by using a process for treating keratin fibres, notably human keratin fibres such as the hair, by applying to said fibres:
i) at least one compound of formula (I) and also the optical or geometrical isomers thereof, the salts thereof, and the solvates thereof, such as the hydrates:

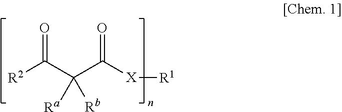

in which formula (I):
R¹ represents a linear or branched, saturated or unsaturated, conjugated or non-conjugated, acyclic or cyclic, aromatic or non-aromatic C₂ to C₁₄, in particular C₃ to C₁₂, polyvalent hydrocarbon-based radical,
R¹ also being:
a) optionally substituted with one or more hydroxyl OH or dialkylamino —N(R)₂ groups with R representing a (C₁-C₄)alkyl group such as methyl, carboxyl —C(O)—OH, a vinyl group, and/or
b) optionally interrupted with one or more heteroatoms or groups chosen from O, S, carbonyl —C(O)—, amine —N(R′), or combinations thereof such as ester —C(O)—O—, —O—C(O)—, amide —C(O)—N(R′)—, —N(R′)—C(O)—, urethane —N(R′)—C(O)—O— or —O—C(O)—N(R′)—, urea —N(R′)—(CO)—N(R′)—, or carbonate —O—C(O)—O—, in which R′ represents a hydrogen atom, a linear or branched alkyl group containing from 1 to 4 carbon atoms optionally substituted with at least one hydroxyl group (OH), or optionally substituted with —X—C(O)—C(Rₐ)(R_{b})—C(O)—R²; preferably, R′ represents a hydrogen atom or a (C₁-C₄)alkyl group;
R² represents a linear or branched, saturated or unsaturated C₁ to C₆ monovalent hydrocarbon-based radical, preferably a (C₁-C₄)alkyl group such as methyl or tert-butyl, more preferentially methyl;
R^{a} and R^{b}, which may be identical or different, represent a hydrogen atom or a (C₁-C₄)alkyl group, preferably hydrogen;
X is a heteroatom or group chosen from O, S, N(R′) or —S—CH₂—C(O)—O—, —O—C(O)—CH₂—S—, —O—C(O)—N(R′)—, —N(R′)—C(O)—O— or —N(R′)—C(O)—N(R′)—; preferably, X represents O;
n denotes an integer ranging from 2 to 10, particularly ranging from 3 to 8; and
ii) at least one crosslinking agent; and
iii) optionally at least one cosmetic active agent.

The inventors have thus found, surprisingly, that the application of ingredients i), ii) and optionally iii) to keratin fibres affords resistance of the effect on the keratin fibres treated with this combination, notably with respect to water and/or to shampoo washing.

For the purposes of the present invention and unless otherwise indicated:
The term “cosmetic active agent” means an organic or organosilicon compound or a mineral compound which can be incorporated into a cosmetic composition to give an effect on keratin fibres, whether this effect is immediate or provided by repeated applications. As examples of cosmetic active agents, mention may be made of coloured or uncoloured, fluorescent or non-fluorescent compounds such as optical brighteners, or UVA and/or UVB screening agents, active agents intended for providing a benefit to keratin fibres, antioxidant active agents, moisturizing active agents, active agents intended for combating the effects of pollution, antimicrobial or bactericidal active agents, fragrances, active agents for caring for keratin fibres such as the hair, and dyestuffs such as direct dyes or pigments, preferably pigments. Preferentially, the cosmetic active agents are chosen from a) dyestuffs chosen from pigments, direct dyes, and mixtures thereof, b) active agents for caring for keratin fibres, preferably the hair, c) UV-screening agents, and d) mixtures thereof.
For the purposes of the present invention, the term “colouring that is persistent with respect to shampoo washing” means that the colouring obtained persists after at least one shampoo wash, particularly after at least three shampoo washes, preferably after at least five shampoo washes.
For the purposes of the present invention, the term “fatty substance” means an organic compound that is insoluble in water at ordinary temperature (25° C.) and at atmospheric pressure (760 mmHg) (solubility of less than 5%, preferably less than 1% and even more preferentially less than 0.1%); in addition, the fatty substances are soluble in organic solvents under the same temperature and pressure conditions, for instance in halogenated solvents such as chloroform or dichloromethane, lower alcohols such as ethanol or aromatic solvents such as benzene or toluene.
an “aryl” radical represents a monocyclic or fused or non-fused polycyclic hydrocarbon-based group comprising from 6 to 14 carbon atoms, and at least one ring of which is aromatic; preferentially, the aryl radical is a phenyl, biphenyl, naphthyl, indenyl, anthracenyl or tetrahydronaphthyl.
a “heteroaryl” radical represents a monocyclic or fused or non-fused polycyclic, 5- to 14-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
the term “(hetero)aryl” means aryl or heteroaryl groups;
the term “(hetero)cycloalkyl” means cycloalkyl or heterocycloalkyl groups;
the “aryl” or “heteroaryl” radicals or the aryl or heteroaryl part of a radical may be substituted with at least one substituent borne by a carbon atom, chosen from:
   a C₁-C₆ and preferably C₁-C₄alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆ and preferably C₁-C₄ alkyl radicals;
   an acylamino radical (—N(R)—C(O)—R′) in which the radical R is a hydrogen atom;
   a C₁-C₄ alkyl radical and the radical R′ is a C₁-C₄ alkyl radical; a carbamoyl radical (R₂N—C(O)—) in which the radicals R, which may be identical or different, represent a hydrogen atom or a C₁-C₄ alkyl radical;
   an alkylsulfonylamino radical (R′—S(O)₂—N(R)—) in which the radical R represents a hydrogen atom or a C₁-C₄ alkyl radical and the radical R′ represents a C₁-C₄ alkyl radical, or a phenyl radical;
   an aminosulfonyl radical (R₂N—S(O)₂—) in which the radicals R, which may be identical or different, represent a hydrogen atom or a C₁-C₄ alkyl radical;
   a carboxylic radical in acid form or salified (preferably with an alkali metal or a substituted or unsubstituted ammonium) form;
   a cyano group (CN);
   a polyhalo(C₁-C₄)alkyl group, preferentially trifluoromethyl (CF₃);
the cyclic or heterocyclic part of a non-aromatic radical may be substituted with at least one substituent borne by a carbon atom, chosen from the groups:
   hydroxyl,
   C₁-C₄ alkoxy, C₂-C₄ (poly)hydroxyalkoxy;
   alkylcarbonylamino (RC(O)—NR′—), in which the radical R′ is a hydrogen atom or a C₁-C₄ alkyl radical and the radical R is a C₁-C₄ alkyl radical, amino substituted with one or two identical or different C₁-C₄ alkyl groups;
   alkylcarbonyloxy (RC(O)—O—), in which the radical R is a C₁-C₄ alkyl radical, amino substituted with one or two identical or different C₁-C₄ alkyl groups;
   alkoxycarbonyl (RO—C(O)—) in which the radical R is a C₁-C₄ alkyl radical;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds, preferably one or more double bonds;
a “cyclic” or “cycloalkyl” radical is a monocyclic or fused or non-fused polycyclic, non-aromatic cyclic hydrocarbon-based radical containing from 5 to 14 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl and dithiolane;
an “alkyl” radical is a linear or branched, in particular C₁-C₆ and preferably C₁-C₄ saturated hydrocarbon-based radical;
an “alkoxy” radical is an alkyl-oxy radical for which the alkyl radical is a linear or branched C₁-C₆ and preferentially C₁-C₄ hydrocarbon-based radical;
a “(poly)(hydroxy)alkyl” radical denotes a C₁-C₆ and preferably C₁-C₄ alkyl radical optionally substituted with one or more hydroxyl radicals, preferably substituted with from 1 to 4 hydroxyl groups, more particularly between 1 and 3;
a “sugar” radical is a monosaccharide or disaccharide radical. Sugar radicals that may be mentioned include: sucrose, glucose, galactose, ribose, fucose, maltose, fructose, mannose, arabinose, xylose and lactose;
the term “monosaccharide” refers to a mono-oside sugar comprising at least 5 carbon atoms of formula Cₓ(H₂O)ₓ with x an integer greater than or equal to 5, preferably x is greater than or equal to 6, in particular x is between 5 and 7 inclusive, preferably x=6, they may be of D or L configuration, and of alpha or beta anomer, and also the salts thereof and the solvates thereof such as hydrates;
the term “disaccharide” refers to a di-oside sugar which is a compound constituted of two saccharides bonded together via O-oside bonds, said compounds being constituted of two monosaccharide units (also known as mono-osides) as defined previously, said monosaccharide units comprising at least 5 carbon atoms, preferably 6; in particular, the mono-oside units are linked together via a 1,4 or 1,6 bond as α (alpha) or β□ (beta) anomer, it being possible for each oside unit to be of L or D configuration, and also the salts thereof and the solvates thereof such as the hydrates of said monosaccharides; more particularly, they are polymers formed from two saccharides (or monosaccharides) having the general formula: —[Cₓ(H₂O)y)]₂— or —[(CH₂O)x]₂—, with x being an integer greater than or equal to 5, preferably x is greater than or equal to 6, in particular x is between 5 and 7 inclusive, preferably x=6, and y is an integer representing x−1;
the term “polysaccharide” refers to a poly-oside sugar which is a polymer constituted of at least 3 saccharides bonded together via O-oside bonds, said polymers being constituted of monosaccharide units (also known as mono-osides) as defined previously, said monosaccharide units comprising at least 5 carbon atoms, preferably 6; in particular, the mono-oside units are linked together via a 1,4 or 1,6 bond as α (alpha) or β□ (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— or —[(CH₂O)ₓ]_{w}—, where x is an integer greater than or equal to 5, preferably x is greater than or equal to 6, in particular x is between 5 and 7 inclusive and preferably x=6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200;
the term “organic or mineral acid salt” more particularly means a) addition salts with an organic or mineral acid in particular chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{∘}—C(O)—OH (I′z), in which formula (I′z) R^{∘} represents a (hetero)aryl group such as phenyl, a (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{∘} preferably denoting a (C₁-C₆)alkyl group optionally substituted with 1, 2 or 3 hydroxyl or carboxyl groups; more preferentially, the monocarboxylic acids of formula (I′z) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid; or b) addition salts with an organic or mineral base such as alkali metal or alkaline-earth metal hydroxides, alkali metal or alkaline-earth metal carbonates, alkali metal or alkaline-earth metal bicarbonates, addition salts with basic amino acids such as arginine or lysine, addition salts with optionally hydroxylated amines;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methanesulfonate or mesylate and ethanesulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Ar—O—S(O)O— such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate;
the “solvates” represent hydrates and also the combination with linear or branched C₁-C₄ alcohols such as ethanol, isopropanol or n-propanol;
the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm).
the term “UV-B screening agent” means a chromophore derived from a compound which screens out (or absorbs) UV-B ultraviolet rays at a wavelength of between 280 and 320 nm.
the term “anhydrous” implies an amount of less than 5% by weight of water, preferentially less than 3% by weight of water, better still less than 1% by weight of water, relative to the total weight of composition C1, C2, C3 or C4 in question; even more preferentially, the composition is free of water;
the terms “colouring agent” and “dyestuff” are equivalent.

Unless otherwise indicated, the limits delimiting the extent of a range of values are included in that range of values.

Unless otherwise indicated, the amounts indicated are expressed as mass percentages.

### Process for Treating Keratin Fibres

The first subject of the invention is a process in one or more steps using, on keratin fibres, notably the hair:
i) at least one compound of formula (I) as defined previously and hereinbelow;
iii) at least one crosslinking agent as defined hereinbelow; and
iii) optionally at least one cosmetic active agent as defined previously and hereinbelow.

According to a particular embodiment of the invention, the process of the invention involves the step of simultaneously applying ingredients i), ii) and optionally iii).

According to another particular embodiment of the invention, the process comprises at least two steps in which ingredients i), and ii) and optionally iii) (the latter ingredient may be present together with ingredient i) and/or with the ingredient(s) ii)) are applied in separate, successive steps to the keratin fibres.

According to a particular embodiment, the ingredient(s) i) are applied to the keratin fibres, and the ingredient(s) ii) are then applied to the keratin fibres, it being understood that the ingredient(s) iii), when they are present, may be applied together with i) and/or ii).

According to another particular embodiment, the ingredient(s) ii) are applied to the keratin fibres, and the ingredient(s) i) are then applied to the keratin fibres, it being understood that the ingredient(s) iii), when they are present, may be applied together with i) and/or ii).

According to a particular embodiment of the invention, the process for treating keratin fibres is a process for caring for, styling and/or colouring keratin fibres, preferably the hair.

According to another embodiment of the invention, the process for treating keratin fibres is a hair dyeing process.

According to one aspect of the invention, the process of the invention for treating keratin fibres using the ingredients i), ii) and optionally iii) is a haircare treatment.

According to another aspect of the invention, the process of the invention for treating keratin fibres using the ingredients i), ii) and optionally iii), is a process for styling and/or dyeing keratin fibres.

According to one embodiment of the invention, the process for treating keratin fibres uses:
a composition, termed “C1”, comprising i) at least one compound of formula (I) or (Ia) as defined previously and hereinbelow, and optionally iii) at least one cosmetic active agent as defined previously and hereinbelow; preferably, composition C1 does not comprise any crosslinking agent;
a composition, termed “C2”, comprising i) at least one compound of formula (I) or (Ia) as defined previously and hereinbelow, and ii) at least one crosslinking agent as defined hereinbelow, and optionally iii) at least one cosmetic active agent as defined previously and hereinbelow;
a composition termed “C3” comprising i) at least one compound of formula (I) or (Ia) as defined previously and hereinbelow, ii) at least one crosslinking agent as defined hereinbelow, and iii) at least one cosmetic active agent as defined previously; and/or
a composition, “C4”, comprising ii) at least one crosslinking agent as defined hereinbelow, and optionally iii) at least one cosmetic active agent as defined previously and hereinbelow; preferably, composition C4 does not comprise any compound of formula (I) or (Ia);
a composition “C5” comprising iii) at least one cosmetic active agent as defined previously and hereinbelow; preferably, composition C5 does not comprise i) any compound of formula (I), or ii) any crosslinking agent as defined hereinbelow;
   it being understood that:
   the process uses together or separately i) at least one compound of formula (I) or (Ia) as defined previously and hereinbelow and ii) at least one crosslinking agent as defined hereinbelow; and
   compositions C1, C2, C3, C4 and C5 may be anhydrous, aqueous such as aqueous-alcoholic and/or comprise one or more fatty substances iv), notably one or more preferably volatile oils as defined hereinbelow.

According to one aspect of the invention, the process for treating keratin fibres, in particular for caring for, styling and/or dyeing keratin fibres, notably the hair, uses a composition, termed “C1”, comprising i) as defined previously and hereinbelow, and optionally iii) at least one cosmetic active agent as defined hereinbelow, notably chosen from a) colouring agents, such as pigments, direct dyes, and mixtures thereof, b) active agents for caring for keratin fibres, c) UV-screening agents, and d) mixtures thereof, and notably at least one colouring agent, more particularly at least one pigment.

According to another aspect, the process of the invention for treating keratin fibres, in particular for caring for, styling and/or dyeing keratin fibres, uses a composition, termed “C2”, comprising i) at least one compound of formula (I) as defined previously and hereinbelow, and ii) at least one crosslinking agent as defined hereinbelow, and optionally iii) at least one cosmetic active agent as defined previously.

According to another aspect, the process of the invention for treating keratin fibres, in particular for caring for, styling and/or dyeing keratin fibres, uses a composition, termed “C3”, comprising i) at least one compound of formula (I) as defined previously and hereinbelow, ii) at least one crosslinking agent as defined hereinbelow, and iii) at least one cosmetic active agent as defined previously.

According to another aspect, the process of the invention for treating keratin fibres, in particular for caring for, styling and/or dyeing keratin fibres, uses a composition termed “C1” and a composition termed “C4”, said composition “C1” comprising i) at least one compound of formula (I) as defined previously and hereinbelow and optionally iii) at least one cosmetic active agent as defined previously, and said composition “C4” comprising ii) at least one crosslinking agent as defined hereinbelow, and optionally iii) at least one cosmetic active agent as defined previously.

According to another variant of the process of the invention, the ingredient(s) i) and optionally iii) are applied together (i.e. simultaneously) to the keratin fibres during a first step, then, during a subsequent step, the ingredient(s) ii) and optionally iii) are applied to said keratin fibres. According to a particular embodiment of the process, in the first step composition “C1” is applied to the keratin fibres, and a composition “C4” comprising ii) at least one crosslinking agent and optionally iii) at least one cosmetic active agent as defined previously is then applied to said fibres.

According to yet another variant of the process of the invention, ingredient(s) ii) and optionally iii) are applied to said keratin fibres, and the ingredient(s) i) and optionally iii) at least one cosmetic active agent as defined previously are then applied to said fibres. In particular, composition C4 is applied to the keratin fibres and composition C1 is then applied.

Compositions C1, C2, C3, C4 and/or C5 of the process of the invention, which comprise at least one fatty substance, notably at least one oil, and water, may be in the form of a direct or inverse emulsion.

Compositions C1, C2, C3, C4 and/or C5 of the process according to the invention may thus be applied directly as such to the target keratin fibres or may even be formed directly on the surface of these keratin fibres.

According to yet another variant of the process of the invention, ingredient ii), notably metal alkoxides and more particularly titanium alkoxides such as titanium butoxide, and optionally iii) are applied to the keratin fibres, and ingredient i) and optionally iii) at least one cosmetic active agent as defined previously are then applied to said fibres. In particular, composition C4 is applied to the keratin fibres and composition C1 is then applied.

According to another variant of the process of the invention, ingredients i), ii) and optionally iii) are applied together during a first step, and ingredient ii), which is identical to or different from ingredient ii) used in the first step, is then applied again to said keratin fibres during a subsequent step. According to a particular embodiment of the process, in the first step a composition “C2” or a composition “C3” is applied to the keratin fibres, and a composition “C4” comprising ii) at least one crosslinking agent and optionally iii) at least one cosmetic active agent as defined previously is then applied to said fibres.

According to the invention, three application methods known as the “one-gesture application mode”, the “two-gesture application mode” and the “three-gesture application mode” are thus distinguished.

According to one embodiment of the process of the invention, it is performed in one gesture by applying composition C2 or C3 as defined previously to the keratin fibres.

The term “one-gesture application mode” means the direct application to the target keratin fibres of a single composition in accordance with the invention, namely composition C2 or C3.

After application of composition C2 or C3, a deposit is advantageously obtained which is persistent with respect to water and/or to shampoo washing, and/or which has good retention of the shape of the keratin fibres, notably of the curls, and/or which makes styling easy and/or gives the keratin fibres a pleasant feel, and which can give body to said fibres. The deposit obtained is also resistant to water and/or to shampoo washing.

According to another embodiment of the process of the invention, it is performed in two gestures.

The term “two-gesture application mode” means the successive application, to the target keratin fibres, of two different compositions, for example C1 and C4, or C2 and C4, or C3 and C4. Preferably C1 and then C4, C2 and then C4, or C3 and then C4. The two-gesture application mode may notably comprise the successive application to the keratin fibres of a composition C2 or C3 and a composition C4, preferably C2 or C3 and then C4.

In the two-gesture application mode, the composition applied first to the keratin fibres, for example C1, is conventionally referred to as the “base coat”, and the composition superposed thereon, for example C4, is generally referred to as the “top coat”.

According to another embodiment, the process of the invention is performed in three gestures.

The term “three-gesture application mode” means the sequential application of three different compositions C1 to C5. According to this application mode, for example, successive application is performed on the keratin fibres, a) of a composition C1, then p) of a composition C4, then y) of a composition C5, preferably C1 then C4 then C5, or C1 then C5 then C4. According to another option, sequential application is performed on the keratin fibres, a) of a composition, for example C4, and p) of a composition C1 or C2, and even y) of a composition C5; preferably, composition C4 is applied before composition C1.

In the two- or three-gesture application modes, the composition applied first, for example C1, is conventionally referred to as the “base coat”, and the composition(s) superposed thereon are generally referred to as the “top coat”.

After application of the various compositions C1 to C5, a deposit is advantageously obtained which is persistent on keratin fibres and/or which has good retention of the shape of the keratin fibres, notably of the curls, and/or which makes styling easy and/or has a pleasant feel. The deposit obtained on said fibres is also resistant notably to water and/or to shampoo washing.

According to a particular embodiment, the compositions are applied to dry keratin fibres after combing and drying naturally, or with a hair dryer, preferably a hairdryer.

According to a particular embodiment, the keratin fibres are dried after application of compositions C1 to C5, in particular after application of each different composition. The drying step may be performed with a drying device such as a hood, a hairdryer or a Climazon. When the drying step is performed with a hood or a hairdryer, the drying temperature is above 40° C.

After the drying step, a step of shaping the keratin fibres may be performed with a straightening iron, a crimping or curling iron or a steam iron, preferably a straightening iron or a steam iron. Preferably, the shaping step is performed at a temperature above 100° C. The iron may be applied to the keratin fibres by separate successive strokes of a few seconds, or by moving or gliding gradually along the hair. Preferably, the iron is applied in a continuous movement from the root to the end of the hair, in one or more passes.

According to one aspect, the present invention relates to a process for dyeing keratin fibres, notably the hair, comprising a step of applying to said hair a composition C2 or C3, notably containing at least one dyestuff notably as defined previously and more particularly at least one pigment.

According to another of its aspects, the present invention relates to a process for dyeing keratin fibres, in particular human keratin fibres, notably the hair, comprising at least the sequential application:
of a composition C1 as defined previously; and then
of a composition C4 as defined previously;
it being understood that compositions C1 and/or C4 contain at least one dyestuff as defined hereinbelow, preferably at least one pigment. Preferably, composition C1 comprises at least one pigment.

According to another of its aspects, the present invention relates to a process for dyeing keratin fibres, in particular human keratin fibres, notably the hair, comprising at least the sequential application:
of a composition C1 as defined previously; and then
of a composition C4 as defined previously; and
of a composition C5 as defined previously;
it being understood that compositions C1 and/or C4 and/or C5 contain at least one dyestuff as defined hereinbelow, preferably at least one pigment. Preferably, composition C5 comprises at least one pigment.

According to another of its aspects, the present invention relates to a process for dyeing keratin fibres, in particular human keratin fibres, notably the hair, comprising at least the sequential application:
of a composition C2 as defined previously; and then
of a composition C4 as defined previously;
it being understood that compositions C2 and/or C4 contain at least one dyestuff as defined hereinbelow, preferably at least one pigment. Preferably, composition C4 comprises at least one pigment.

According to yet another of its aspects, the present invention relates to a process for dyeing keratin fibres, in particular human keratin fibres, notably the hair, comprising at least the sequential application:
of a composition C2 as defined previously;
of a composition C4 as defined previously; and
of a composition C5 as defined previously;
it being understood that compositions C2 and/or C4 and/or C5 contain at least one dyestuff as defined hereinbelow, preferably at least one pigment. Preferably, composition C5 comprises at least one pigment.

According to another of its aspects, the present invention relates to a process for making up keratin materials, in particular keratin fibres, notably human keratin fibres, in particular the hair, comprising at least the sequential application:
of a composition C1, C2 or C3 as defined previously; and
of a composition C4 as defined previously;
it being understood that at least one of the compositions comprises at least one cosmetic active agent iii) chosen from dyestuffs, preferably from pigments.
   i) Compound of formula (I)

The first ingredient of the process of the invention is the compound of formula (I) as defined previously.

According to a particular embodiment of the invention, ingredient i) is chosen from those of formula (Ia) and also the optical isomers or geometrical isomers thereof, the salts thereof, and the solvates thereof, such as hydrates:

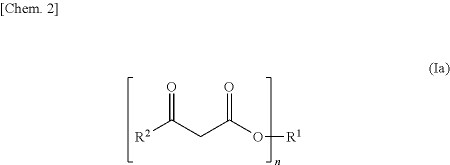

in which formula (Ia):
R¹ is as defined previously, and particularly represents a linear or branched, saturated or unsaturated, conjugated or non-conjugated, acyclic or cyclic, aromatic or non-aromatic C₂ to C₁₄, preferably C₃ to C₁₂ and preferably C₄ to C₁₀ polyvalent hydrocarbon-based radical, R¹ also being:
a) optionally substituted with one or more hydroxyl groups OH, and/or
b) optionally interrupted with one or more heteroatoms or groups chosen from O, S, —N(R′), preferably interrupted with one or more oxygen atoms, in which R′ is as defined in formula (I);
R² is as defined previously, preferably R² represents a linear or branched (C₁-C₆)alkyl group, preferably a (C₁-C₄)alkyl group, particularly methyl or tert-butyl, such as methyl; and
n being as defined previously; preferably, n denotes an integer ranging from 2 to 8.

According to one embodiment, the compounds of formulae (I) and (Ia) are such that R1 represents a linear or branched, unsaturated monocyclic or bicyclic, preferably monocyclic, C6 to C8 polyvalent hydrocarbon-based radical, and an aromatic radical such as phenyl.

According to another embodiment, the compounds of formulae (I) and (Ia) are such that R1 represents a saturated monocyclic or bicyclic C5 to C12 polyvalent hydrocarbon-based radical, optionally interrupted with one or more heteroatoms or groups chosen from O, S and —N(R′)—, in which R′ represents a hydrogen atom or an alkyl group containing from 1 to 4 carbon atoms, preferably interrupted with one or more oxygen atoms. Preferably, R1 represents a monosaccharide or polysaccharide unit, notably a disaccharide, on which n hydroxyl groups have been substituted with n groups —O—C(O)—CH2-C(O)—R2, with R2 as defined previously and n as defined previously.

Suitable monosaccharides according to the invention include, but are not limited to, ribose, glucose, mannose, galactose, fructose and sorbose.

According to one embodiment, the disaccharides are chosen from cellobiose, maltose, lactose, raffinose, sucrose, trehalose, melibiulose, melibiose, mannobiose, kojibiose, nigerose, isomaltose, rutinose, rutinulose and xylobiose.

According to another preferred embodiment of the invention, the compounds of formulae (I) and (Ia) are such that R1 represents a linear or branched, saturated or unsaturated, preferably saturated, acyclic C2-C12, preferably C3-C10, polyvalent hydrocarbon-based radical.

According to a particular embodiment of the invention, the compounds of formulae (I) and (Ia) are such that n denotes an integer from 2 to 8; more particularly, n denotes an integer from 3 to 6, more particularly from 3 to 5. According to one embodiment, the compounds of formulae (I) and (Ia) are such that n is 2. According to another embodiment, the compounds of formulae (I) and (Ia) are such that n is 3. According to another embodiment, the compounds of formulae (I) and (Ia) are such that n is 4. According to another embodiment, the compounds of formulae (I) and (Ia) are such that n is 5. According to another embodiment, the compounds of formulae (I) and (Ia) are such that n is 6.

In one variant of the invention, the compound of formula (I) or (Ia) as defined previously is a mixture of compounds of different chemical structures.

More particularly, the compound(s) of formulae (I) and (Ia) are chosen from the following compounds, and also the optical isomers thereof, the salts thereof, and the solvates thereof, such as hydrates:

| (Compound) | | |
|---|---|---|
| CAS No. | Chemical name | Chemical structure |
| (1) 700865-73-2 | butanoic acid 3-oxo- 1,1′-(1,7-heptanediyl) ester | 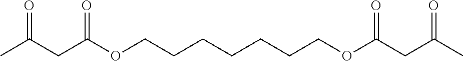 [SMILES: CC(=O)CC(=O)OCCCCCCCOC(=O)CC(C)=O] |
| (2) 632336-20-0 | butanoic acid 3-oxo- 1,1′-(1,8-octanediyl) ester | 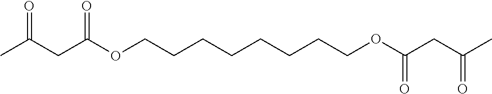 [SMILES: CC(=O)CC(=O)OCCCCCCCCOC(=O)CC(C)=O] |
| (3) 501426-98-8 | butanoic acid 3-oxo- 1,1′-(1,10-decanediyl) ester | 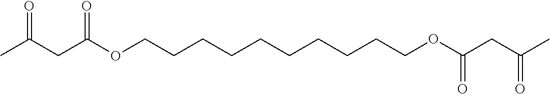 [SMILES: CC(=O)CC(=O)OCCCCCCCCCCOC(=O)CC(C)=O] |
| (4) 174498-06-7 | butanoic acid 3-oxo- 1,1′-(1,3-propanediyl) ester | 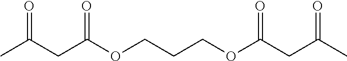 [SMILES: CC(=O)CC(=O)OCCCOC(=O)CC(C)=O] |
| (5) 160187-52-0 | butanoic acid 3-oxo- 1,2-dimethyl-1,2- ethanediyl ester | 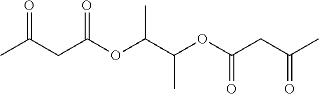 [SMILES: CC(=O)CC(=O)OC(C)C(C)OC(=O)CC(C)=O] |
| (6) 145020-19-5 | butanoic acid 3-oxo- 2-butyl-2-ethyl-1,3- propanediyl ester | 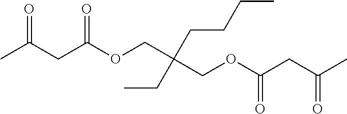 [SMILES: CCCCC(CC)(COC(=O)CC(C)=O)COC(=O)CC(C)=O] |
| (7) 87530-36-7 | butanoic acid 3-oxo- 1,1,2,2-tetramethyl- 1,2-ethanediyl ester | 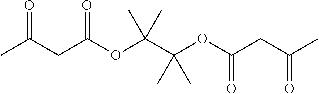 [SMILES: CC(=O)CC(=O)OC(C)(C)C(C)(C)OC(=O)CC(C)=O] |
| (8) 58213-75-5 | butanoic acid 3-oxo- 1,1′-(1-methyl-1,3- propanediyl)ester | 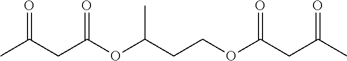 [SMILES: CC(=O)CC(=O)OCCC(C)OC(=O)CC(C)=O] |
| (9) 58213-74-4 | butanoic acid 3-oxo- 1,1′-[2,2-dimethyl-1- (1-methylethyl)-1,3- propanediyl]ester | 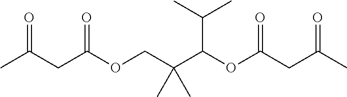 [SMILES: CC(=O)CC(=O)OCC(C)(C)C(OC(=O)CC(C)=O)C(C)C] |
| (10) 39564-28-8 | butanoic acid 3-oxo- 1,1′-(1,5-pentanediyl) ester | 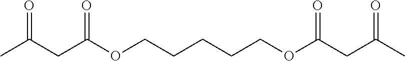 [SMILES: CC(=O)CC(=O)OCCCCCOC(=O)CC(C)=O] |
| (11) 14276-67-6 | butanoic acid 3-oxo- 1,1′-(2,2-dimethyl-1,3- propanediyl)ester | 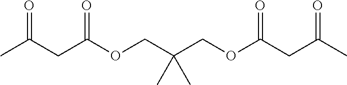 [SMILES: CC(=O)CC(=O)OCC(C)(C)COC(=O)CC(C)=O] |
| (12) 13018-41-2 | butanoic acid 3-oxo- 1,4-butanediyl ester | 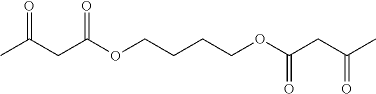 [SMILES: CC(=O)CC(=O)OCCCCOC(=O)CC(C)=O] |
| (13) 6079-90-9 | butanoic acid 3-oxo- 1-methyl-1,2- ethanediyl ester | 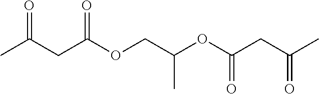 [SMILES: CC(=O)CC(=O)OCC(C)OC(=O)CC(C)=O] |
| (14) 5459-04-1 | butanoic acid 3-oxo- 1,2-ethanediyl ester | 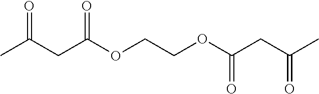 [SMILES: CC(=O)CC(=O)OCCOC(=O)CC(C)=O] |
| (15) 2388-18-3 | butanoic acid 3-oxo- 1,1′-(1,6-hexanediyl) ester butanoic acid 3-oxo- 1,6-hexanediyl ester (9CI) | 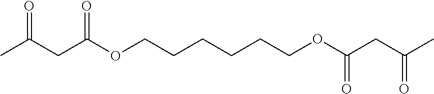 [SMILES: CC(=O)CC(=O)OCCCCCCOC(=O)CC(C)=O] |
| (16) 32818-60-3 | 1,1′-[2,2-bis[(1,3- dioxobutoxy)methyl]- 1,3-propanediyl] bis(3-oxobutanoate) | 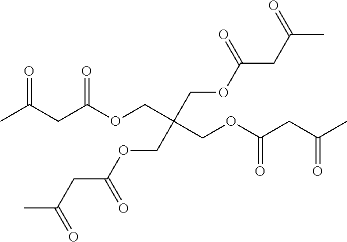 [SMILES: CC(=O)CC(=O)OCC(COC(=O)CC(C)=O)(COC(=O)CC(C)=O)COC(=O)CC(C)=O] |
| (17) 51980-20-2 | 1,1′-[2,2- bis(hydroxymethyl)- 1,3-propanediyl] bis(3-oxobutanoate) | 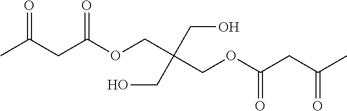 [SMILES: CC(=O)CC(=O)OCC(CO)(CO)COC(=O)CC(C)=O] |
| (18) | | 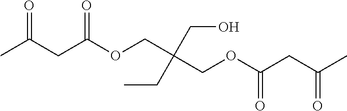 [SMILES: CCC(CO)(COC(=O)CC(C)=O)COC(=O)CC(C)=O] |
| (19) 130973-05-6 | butanoic acid 3-oxo- 1,3,5-benzenetriyl ester | 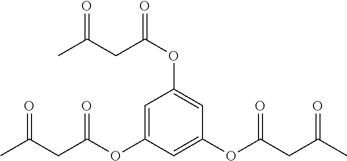 [SMILES: CC(=O)CC(=O)Oc1cc(OC(=O)CC(C)=O)cc(OC(=O)CC(C)=O)c1] |
| (20) 139264-86-1 | butanoic acid 3-oxo- 1,2,4-benzenetriyl ester | 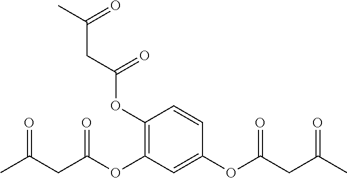 [SMILES: CC(=O)CC(=O)Oc1ccc(OC(=O)CC(C)=O)c(OC(=O)CC(C)=O)c1] |
| (21) 22208-25-9 | trimethylolpropane triacetoacetate K-FLEX 7301 | 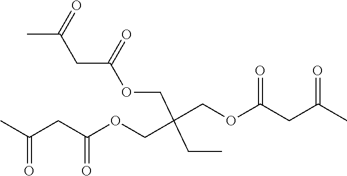 [SMILES: CCC(COC(=O)CC(C)=O)(COC(=O)CC(C)=O)COC(=O)CC(C)=O] |
| (22) 2360524-48- 5 | — | 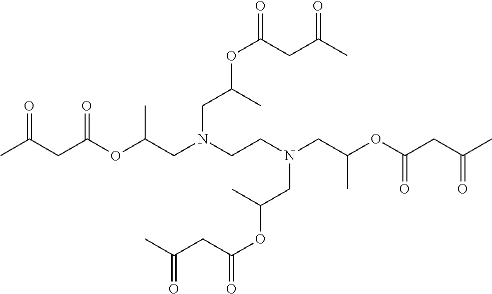 [SMILES: CC(=O)CC(=O)OC(C)CN(CCN(CC(C)OC(=O)CC(C)=O)CC(C)OC(=O)CC(C)=O)CC(C)OC(=O)CC(C)=O] |
| (23) 66229-33-2 | butanoic acid 3-oxo- 2-butyl-2-[(1,3- dioxobutoxy)methyl]- 1,3-propanediyl ester | 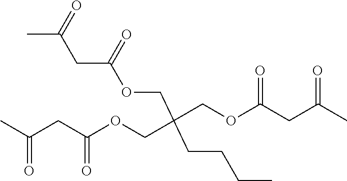 [SMILES: CCCCC(COC(=O)CC(C)=O)(COC(=O)CC(C)=O)COC(=O)CC(C)=O] |
| (24) 1579231-50- 7 | pentanoic acid 4,4- dimethyl-3-oxo-, 1,1′- [2,2-bis[[(4,4- dimethyl-1,3- dioxopentyl)oxy] methyl]-1,3- propanediyl]ester | 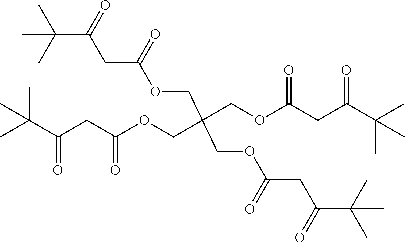 [SMILES: CC(C)(C)C(=O)CC(=O)OCC(COC(=O)CC(=O)C(C)(C)C)(COC(=O)CC(=O)C(C)(C)C)COC(=O)CC(=O)C(C)(C)C] |
| (25) 6079-98-7 | 1,1′,1″-(1,2,3- propanetriyl)tris(3- oxobutanoate) | 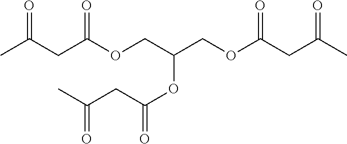 [SMILES: CC(=O)CC(=O)OCC(COC(=O)CC(C)=O)OC(=O)CC(C)=O] |
| (26) — | meso-erythrityl tetraacetoacetate | 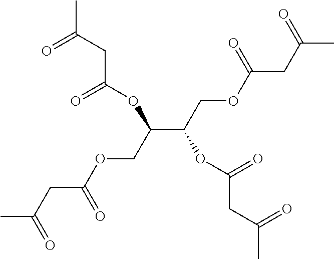 [SMILES: CC(=O)CC(=O)OC[C@H](OC(=O)CC(C)=O)[C@@H](COC(=O)CC(C)=O)OC(=O)CC(C)=O] |
| (27) 169533-29-3 | α-D-glucopyranose, 1,2,3,4,6-pentakis(3- oxobutanoate) | 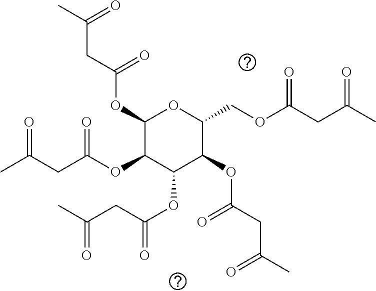 [SMILES: CC(=O)CC(=O)OC[C@H]1O[C@H](OC(=O)CC(C)=O)[C@H](OC(=O)CC(C)=O)[C@@H](OC(=O)CC(C)=O)[C@@H]1OC(=O)CC(C)=O] |
| (28) 2254710-55- 7 | benzoic acid 5-amino- 2-[[2,3,4-tris-O-(1,3- dioxobutyl)-D- xylopyranosyl]oxy]- | 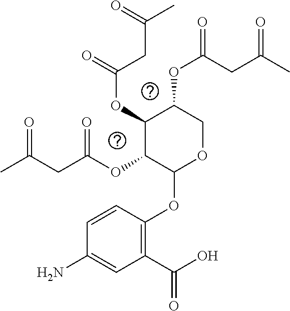 [SMILES: CC(=O)CC(=O)O[C@H]1[C@H](OC(=O)CC(C)=O)COC(Oc2ccc(N)cc2C(=O)O)[C@@H]1OC(=O)CC(C)=O] |
| (29) 1236300-10- 9 | 1,3,4,6-tetrakis-O- (1,3-dioxobutyl)-β-D- fructofuranosyl 2,3,4,6-tetrakis(3- oxobutanoate) α-D- glucopyranoside | 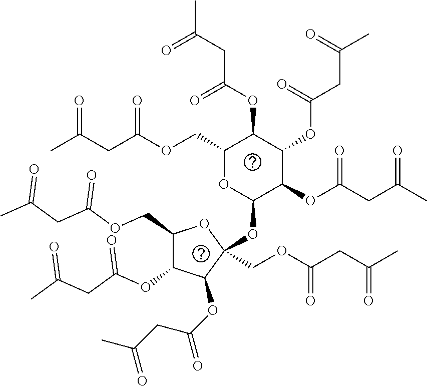 [SMILES: CC(=O)CC(=O)OC[C@H]1O[C@@](COC(=O)CC(C)=O)(O[C@H]2O[C@H](COC(=O)CC(C)=O)[C@@H](OC(=O)CC(C)=O)[C@H](OC(=O)CC(C)=O)[C@H]2OC(=O)CC(C)=O)[C@@H](OC(=O)CC(C)=O)[C@@H]1OC(=O)CC(C)=O] |
| (30) 96481-26-4 | D-glucopyranose pentakis(3- oxobutanoate) | 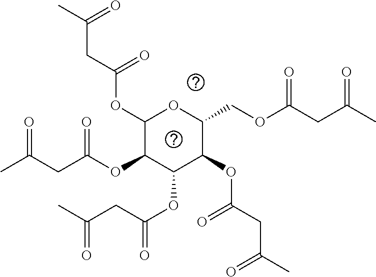 [SMILES: CC(=O)CC(=O)OC[C@H]1OC(OC(=O)CC(C)=O)[C@H](OC(=O)CC(C)=O)[C@@H](OC(=O)CC(C)=O)[C@@H]1OC(=O)CC(C)=O] |
| (31) 944473-12-5 | D-glucitol, 1,4:3,6- dianhydro-, 2,5-bis(3- oxobutanoate) | 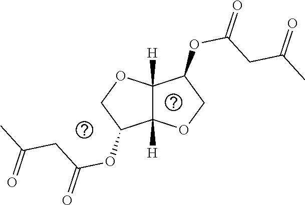 [SMILES: CC(=O)CC(=O)O[C@H]1CO[C@H]2[C@@H]1OC[C@H]2OC(=O)CC(C)=O] |
| (32) 2254710-49- 9 | — | 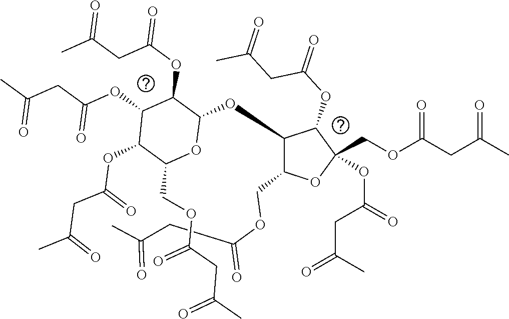 [SMILES: CC(=O)CC(=O)OC[C@H]1O[C@@H](O[C@@H]2[C@@H](COC(=O)CC(C)=O)O[C@@](COC(=O)CC(C)=O)(OC(=O)CC(C)=O)[C@H]2OC(=O)CC(C)=O)[C@H](OC(=O)CC(C)=O)[C@@H](OC(=O)CC(C)=O)[C@H]1OC(=O)CC(C)=O] |
| (33) — | sorbitol hexaacetoacetate | 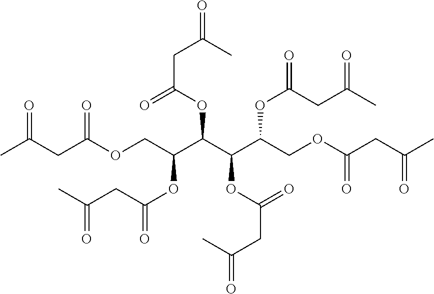 [SMILES: CC(=O)CC(=O)OC[C@H](OC(=O)CC(C)=O)[C@@H](OC(=O)CC(C)=O)[C@H](OC(=O)CC(C)=O)[C@@H](COC(=O)CC(C)=O)OC(=O)CC(C)=O] |
| (34) 216756-89-7 | D-glucitol, 4-O-β-D- galactopyranosyl-, polyacetoaceate | 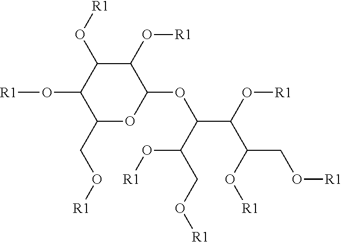 |
| | | with R1 chosen from —C(O)—CH₂— |
| | | C(O)—CH₃, H |
| | | with at least two radicals R1 |
| | | representing —C(O)—CH₂—C(O)—CH₃ |
| (35) | sucrose polyacetoacetate | 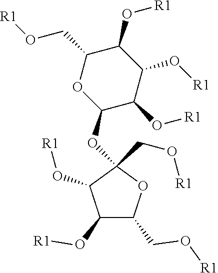 |
| | | with R1 chosen from —C(O)—CH₂—C(O)—CH₃, H |
| | | with at least two radicals R1 representing |
| | | —C(O)—CH₂—C(O)—CH₃ |
| (36) | glucose polyacetoacetate | 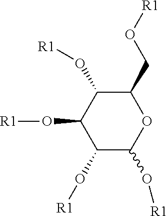 |
| (37) | mannose polyacetoacetate | 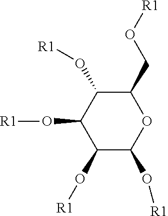 |
| (38) | xylitol polyacetoacetate | 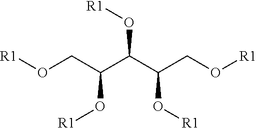 |
| (39) | sorbitol polyacetoacetate | 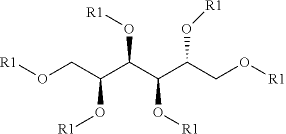 |
| | | preferably six radicals R1 representing |
| | | —C(O)—CH₂—C(O)—CH₃ |
| (40) | | 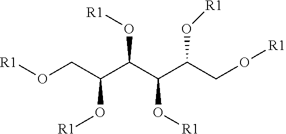 |
| | | three radicals R1 representing —C(O)—CH₂—C(O)—CH₃, |
| | | and three radicals R1 representing a |
| | | hydrogen atom |
| (41) | 1471-94-9 | 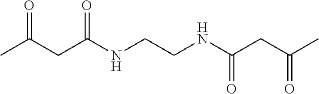 [SMILES: CC(=O)CC(=O)NCCNC(=O)CC(C)=O] |
| (42) | 2552-36-5 | 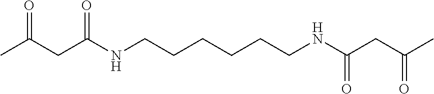 [SMILES: CC(=O)CC(=O)NCCCCCCNC(=O)CC(C)=O] |
| (43) | 139614-56-5 | 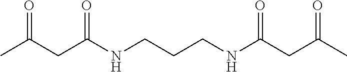 [SMILES: CC(=O)CC(=O)NCCCNC(=O)CC(C)=O] |
| (44) | 96169-30-1 | 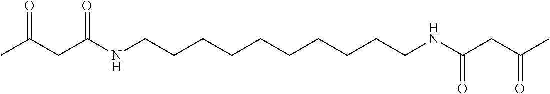 [SMILES: CC(=O)CC(=O)NCCCCCCCCCCNC(=O)CC(C)=O] |
| (45) | 75639-01-9 | 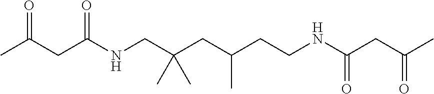 [SMILES: CC(=O)CC(=O)NCCC(C)CC(C)(C)CNC(=O)CC(C)=O] |
| (46) | dipentaerythritol polyacetoacetate | 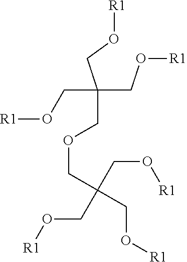 |
| | | preferably five or six radicals R1, more |
| | | preferentially six radicals R1 representing |
| | | —C(O)—CH₂—C(O)—CH₃ |

in which R1, which may be identical or different, represents a hydrogen atom or a —C(O)—CH₂—C(O)—CH₃ group, it being understood that at least two radicals R1 represent —C(O)—CH₂—C(O)—CH₃, preferably at least three radicals R1 represent —C(O)—CH₂—C(O)—CH₃.
   More preferentially, the compound(s) of formulae (I) and (Ia) are chosen from compounds (16), (18), (21), (35), (36), (37), (38), (39), (40) and (46) and better still (16), (21), (35), (38), (39) and (46).
   Even more preferentially, the compound(s) of formulae (I) and (Ia) are chosen from the compounds (16), (18), (21), (35), (36), (37), (38), (39) and (40) and better still (16), (21), (35), (38) and (39).
   Advantageously, the compound(s) of formula (I) and (Ia) are chosen from the compounds (18), (21), (36), (37), (38), (39) and (40) and better still (21), (38) and (39).

The compound(s) of formula (I) or (Ia) as defined previously are preferably present in a mass ratio of from 0.2% to 100%, particularly from 0.5% to 50%, more particularly from 2% to 35% relative to the total weight of composition C1, C2 or C3 containing same. In particular, the crosslinking agent(s) and the compound(s) of formula (I) or (Ia) are preferably present in a mass ratio of from 7% to 35% relative to the total weight of the composition comprising same.

The compound(s) of formula (I) may be obtained by (poly)condensation(s) of n equivalents of dicarbonyl reagent with a nucleophilic reagent R¹(XH)ₙ including n equivalents of nucleophilic functions —XH according to the following scheme:

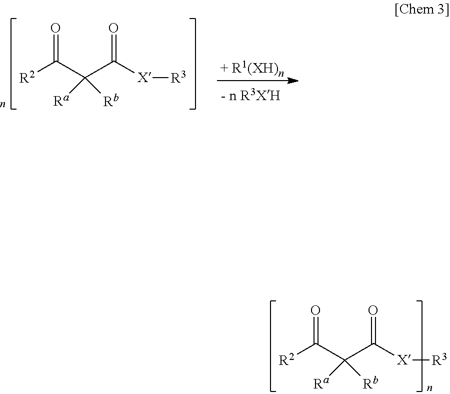

in which scheme:
R¹, R², R^{a}, R^{b} and n are as defined for the compound of formula (I); and
R³ represents a hydrogen atom, a (C₁-C₄)alkyl group such as methyl, ethyl, isobutyl, t-butyl, or an electrofugal group such as CHaI₃, or Hal₃C—SO₂— such as triflate, with Hal, which may be identical or different, representing a halogen atom;
X′ represents an oxygen or sulfur atom, preferably O.

These (poly)condensation methods are known to those skilled in the art: mention may be made, for example, of “From rigid and flexible foams to elastomers via Michael addition chemistry”, Polymer, 106, 128-139, (2016), “Dynamic Curing Agents for Amine-Hardened Epoxy Vitrimers with Short (Re)processing Times”, Macromolecules, 53(7), 2485-2495 (2020), “Super photo-base initiated organic-inorganic hybrid coatings by plural-cure mechanisms”, Progress in Organic Coatings, 127, 222-230 (2019) or patent application PL159100: Method for manufacturing acetylacetate esters.

According to a particular embodiment of the invention, R1(XH)n is chosen from acyclic polyols R1(OH)n notably chosen from glycerol, trimethylolpropane, ethylene glycol, diethylene glycol, triethylene glycol, tetraethylene glycol, propylene glycol, dipropylene glycol, butylene glycol, trimethylolethane, pentamethylene glycol, trimethylpentanediol, pentyl glycol, isosorbide, pentaerythritol, dipentaerythritol, hexamethylene glycol, hexylene glycol, hexanediol, neopentyl glycol, 1,2-propanediol; 1,3-propanediol; 1,2-butanediol, 1,3-butanediol, 2,3-butanediol, 1,4-butanediol; 2,4-butanediol, 3,4-butanediol; 1,4-pentanediol; 1,5-pentanediol, 2,2,4-trimethyl-1,3-pentanediol, 1,6-hexanediol, 1,2-octanediol, 1,8-octanediol, 1,10-decanediol, 2-methyl-1,3-propanediol, hexylene glycol and isoprene glycol.

In one variant, R1(XH)n is chosen from cyclic polyols R1(OH)n notably chosen from monosaccharides and disaccharides, as defined previously, alone or as mixtures.

Preferentially, R1(XH)n is chosen from polyols R1(OH)n alone or as mixtures, notably from trimethylolethane, glycerol, trimethylolpropane, pentaerythritol, glycerol, glucose, sucrose and sorbitol.

According to another variant, R1(XH)n is chosen from diamines R1(NH₂)₂ notably hydroxyalkylated aliphatic diamines such as o,o′-bis(diethanolaminomethyl)-p-nonylphenol, N,N,N,N′-tetrakis(2-hydroxypropyl)ethylenediamine (Quadrol L, available from BASF) and N,N,N,N-tetrakis(2-hydroxyethyl)ethylenediamine, and mixtures thereof.

According to another advantageous variant, the compound(s) of formula (I) or (Ia) are obtained by (poly)condensation(s) of n equivalents of propylenedioxy carbonyl reagent with a polyamine nucleophilic reagent R1(NH)n including n equivalents of —NH₂ nucleophilic functions according to the following scheme:

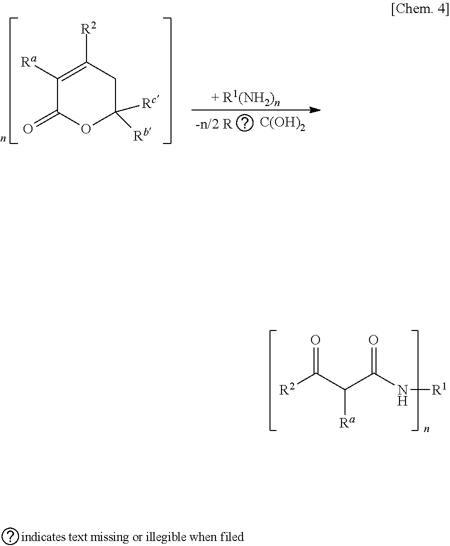

in which scheme:
R¹, R², Rₐ and n are as defined for the compound of formula (I);
R¹—NH₂ is notably chosen from ethylenediamine, 1,3-diaminopropane, cadaverine, hexamethylenediamine, 1,2-diaminopropane, 1,2-diaminocyclohexane and phenylenediamine; and
R^{b′} and R^{c′}, which may be identical or different, represent a hydrogen atom or a (C₁-C₄)alkyl group.

This type of synthetic process according to scheme 2 is known to those skilled in the art: for example, mention may be made of “Acetoacetylation with 2,2,6-trimethyl-4H-1,3-dioxin-4-one: a convenient alternative to diketene”, Journal of Organic Chemistry, 50(14), 2431-5, (1985) or “Synthesis of new Biginelli polycondensates: renewable materials with tunable high glass transition temperatures”, Polymer International, 70(5), 506-513 (2021).

According to another advantageous variant, the compound(s) of formula (I) or (Ia) are obtained by (poly)condensation(s) of n equivalents of diketene reagent with a nucleophilic reagent R1(X)n including n equivalents of nucleophilic functions —XH according to the following scheme:

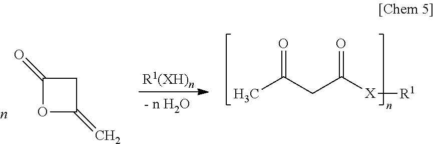

in which scheme:
R¹ and n are as defined for the compound of formula (I);
X′ represents an oxygen or sulfur atom, or N(R′) with R′═H.

This type of process is known to those skilled in the art. Mention may be made, for example, of “Acetoacetamides, 2-hydroximinoacetoacetamides, and 2,3-bis(hydroximino)butyramides”, Journal fur Praktische Chemie (Leipzig), 323(2), 337-44, (1981) or Angewandte Makromolekulare Chemie, 9, 96-105, (1969).

### ii) Crosslinking Agent

The process of the invention uses ii) one or more crosslinking agents.

For the purposes of the invention, the term “crosslinking agent”, also termed “R”, denotes a compound that is capable of establishing with at least one ACAC function of the compound of formula (I) or (Ia):
at least one covalent bond,
at least one donor-acceptor (dative) bond, and/or
at least one coordination bond
and thus of crosslinking the compound of formula (I) or (Ia).

Preferably, the term “crosslinking agent” denotes a compound that is capable of establishing at least one covalent bond with a function ACAC of the compounds of formula (I) or (Ia).

For the purposes of the present invention, it is understood that the terms “crosslinking agent” and “crosslinker” are equivalent.

Compositions C2, C3 and C4 contain at least one crosslinking agent. The compositions of the invention may comprise a fatty phase, an aqueous phase or may be in the form of a direct or inverse emulsion. Composition C4 may be an aqueous composition.

According to one aspect, the process of the invention uses a composition termed “C3”, notably a cosmetic composition for keratin fibres, in particular for caring for, styling and/or colouring keratin fibres, comprising i) at least one compound of formula (I) as defined previously, ii) at least one crosslinking agent and iii) at least one cosmetic active agent as defined previously, and notably at least one colouring agent, more particularly at least one pigment.

Compositions C2, C3 and C4 may be obtained by mixing an aqueous dispersion and/or a composition comprising at least one crosslinking agent R, and at least one cosmetic active agent chosen from a) to d) as defined previously.

The crosslinking agent(s) as defined previously are preferably present in a mass ratio of from 0.2% to 60%, particularly from 0.5% to 40% and more particularly from 5% to 40% relative to the total weight of the composition containing same. In particular, the crosslinking agent(s) and the compound(s) of formula (I) or (Ia) are preferably present in a mass ratio of from 7% to 35% relative to the total weight of the composition comprising same.

More precisely, the crosslinking agent(s) R that are suitable for use in the invention may be chosen from compounds bearing amine, thiol, acrylate and/or carbonyl functions, such as a ketone or aldehyde function. A crosslinking agent R may also denote a metal alkoxide.

Thus, according to a particular embodiment, the crosslinking agent R is chosen from (poly)amine, (poly)thiol, (poly)carbonyl, (poly)acrylate and/or metal alkoxide compounds, and mixtures thereof, and preferably chosen from (poly)amine, (poly)thiol and (poly)acrylate compounds, metal alkoxides, and mixtures thereof.

The term “(poly)amine, (poly)thiol, (poly)carbonyl and (poly)acrylate compounds” is intended to denote compounds including at least one primary or secondary amine, thiol, carbonyl (such as a ketone or aldehyde function) or acrylate function, respectively.

### A) (Poly)Amine Compounds

According to a preferred embodiment, the crosslinking agent R is chosen from (poly)amine compounds.

The (poly)amine compound may be chosen in particular from polyamine compounds bearing several primary and/or secondary amine groups or from amino alkoxysilanes, and more particularly from amino alkoxysilane compounds, diamine compounds, triamine compounds, and mixtures thereof.

The (poly)amine compound may be a compound comprising from 2 to 20 carbon atoms, notably a nonpolymeric compound; they may be acyclic or cyclic, linear or branched, saturated or unsaturated, conjugated or non-conjugated, aromatic or non-aromatic, optionally interrupted with one or more heteroatoms chosen from O, S, Si(R′)₂, N(R″) preferably O, Si(R′)₂, or combinations thereof such as —Si(R′)₂—O— or —O—Si(R′)₂—, with R′, which may be identical or different, representing a (C₁-C₄)alkyl group such as methyl, and R″ representing a hydrogen atom or a (C₁-C₄)alkyl group, preferably hydrogen.

The term “non-polymeric compound” means a compound which is not directly obtained via a monomer polymerization reaction.

(Poly)amine compounds that may be mentioned in particular include N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine, 1,3-bis(aminomethyl)cyclohexane, 1,4-bis(aminomethyl)cyclohexane, diaminopropanol, 4,7,10-trioxa-1,13-tridecanediamine, spermidine and C36-alkylenediamines (Priamine™ 1071, 1073, 1074 and 1075, respectively, sold by Croda).

According to a particular embodiment of the invention, the (poly)amine compound(s) are monoamine compounds, i.e. they contain only one primary and/or secondary amine group, preferably a primary amine group (NH2).

The (poly)amine compound(s) may be chosen from amino alkoxysilanes, notably of formula R′₁Si(OR′₂)_{z}(R′₃)ₓ in which:
R′₁ is a linear or branched, saturated or unsaturated, cyclic or acyclic C1-C6 hydrocarbon-based chain substituted with a group chosen from primary amine groups NH₂ or secondary amine groups —N(H)R with R representing a C₁-C₄ alkyl, an aryl or a benzyl substituted with an amino group or with a C₁-C₄ aminoalkyl group; R′₁ may be interrupted in its chain with a heteroatom (O, S, NH) or a carbonyl group (CO), R′₁ being linked to the silicon atom directly via a carbon atom,
R′₂ and R′₃, which may be identical or different, represent a linear or branched (C₁-C₆)alkyl group,
z denotes an integer ranging from 1 to 3, and
x denotes an integer ranging from 0 to 2,
with z+x=3.

In particular, R′1 is an acyclic chain. Preferably, R′1 is a linear or branched, saturated or unsaturated C1-C6 hydrocarbon-based chain substituted with an amine —NH2 or —N(H)R group, with R representing a C1-C6 alkyl, a C3-C6 cycloalkyl or a C6 aromatic group. More preferentially, R′1 is a saturated linear C1-C6 hydrocarbon-based chain substituted with an amine group NH2. Even more preferentially, R′1 is a saturated linear C2-C4 hydrocarbon-based chain substituted with an amine group NH2.

In particular, R′2 represents an alkyl group comprising from 1 to 4 carbon atoms; preferably, R′2 represents a linear alkyl group comprising from 1 to 4 carbon atoms and more preferentially R′2 represents an ethyl group.

In particular, R′3 represents an alkyl group comprising from 1 to 4 carbon atoms; preferably, R′3 represents a linear alkyl group comprising from 1 to 4 carbon atoms and more preferentially R′3 represents methyl or ethyl groups. Preferably, z is equal to 3.

In particular, the (poly)amine compound(s) are chosen from amino alkoxysilanes including only one primary and/or secondary, preferably primary (NH2), amine group, such as 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, and N-(2-aminoethylaminomethyl)phenethyltrimethoxysilane.

Preferably, the (poly)amine compound(s) are chosen from 3-aminopropyltriethoxysilane (APTES), 3-aminoethyltriethoxysilane (AETES), 3-aminopropylmethyldiethoxysilane, and N-(2-aminoethyl)-3-aminopropyltriethoxysilane, and more preferentially 3-aminopropyltriethoxysilane (APTES) or oligomers thereof.

The (poly)amine compound may also be chosen from amino polymers, notably having a weight-average molecular weight ranging from 500 g·mol−1 to 1 000 000 g·mol−1, preferably ranging from 500 g·mol−1 to 500 000 g·mol−1, and preferentially ranging from 500 g·mol−1 to 100 000 g·mol−1.

According to a particular embodiment of the invention, the (poly)amine compound(s) are monoamine compounds and are chosen from polydialkylsiloxanes, notably of formula (IV)

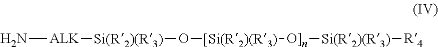

in which formula (IV) ALK represents a linear or branched, preferably linear, (C₁-C₄)alkylene group such as propylene, R′₂ and R′₃, which may be identical or different, preferably identical, represent a (C₁-C₄)alkyl group such as methyl, and R′₄ represents a linear or branched (C₁-C₆)alkyl group, preferably a C₄ group such as n-butyl, n represents an integer greater than or equal to 2; preferably, the value of n is such that the weight-average molecular weight of the polydimethylsiloxane ranges from 500 to 3000 g·mol⁻¹. As examples of polydimethylsiloxanes (IV), mention may be made of the products sold under the names MCR-A11 and MCR-A12 by the company Gelest.

According to a particular embodiment of the invention, the (poly)amine compounds are diamine compounds, i.e. they contain two primary and/or secondary amine groups, preferably primary amine groups (NH₂). More particularly, they are chosen from the compounds of formulae (V) and (VI):

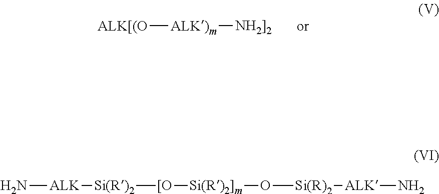

in which formulae (V) and (VI) ALK and ALK′, which may be identical or different, represent a linear or branched, preferably linear, (C₁-C₆)alkylene group, such as propyl, R′, which may be identical or different, represents a (C₁-C₄)alkyl group, such as methyl, m representing an integer greater than or equal to 0; preferably, the value of m is such that the weight-average molecular weight of compound (V) or (VI) ranges from 500 g·mol⁻¹ to 55 000 g·mol⁻¹. As examples of compounds of formula (VI), mention may be made of those sold under the names DMS-A11, DMS-A12, DMS-A15, DMS-A21, DMS-A31, DMS-A32 and DMS-A35 by the company Gelest.

The (poly)amines compounds that are diamines are particularly polyether diamines notably of formula H₂N-ALK-O-[ALK′-O]m-ALK″-NH₂ with ALK, ALK′ and ALK″, which may be identical or different, representing a linear or branched (C₁-C₆)alkylene group, and m representing an integer greater than or equal to 0, such as 4,7,10-trioxa-1,13-tridecanediamine or the compounds known in particular under the reference Jeffamine from the company Hunstman, and more particularly α,ω-diamino polyethylene glycol and/or polypropylene glycol (with an amine function at the end of the chain) such as the products sold under the names Jeffamine D-230, D-400, D-2000, D-4000, ED-600, ED-9000 and ED-2003.

According to a particular embodiment of the invention, the (poly)amine compound(s) are triamine compounds, i.e. they contain three primary and/or secondary amine groups, preferably primary amine groups (NH2). More particularly, the polyether triamines notably of formula ALK′″[(O-ALK′)m-NH2]3 with ALK′ as defined previously and ALK′″ representing a linear or branched trivalent (C1-C6)alkylene group, and m representing an integer greater than or equal to 0.

As (poly)amine compounds that are triamine compounds, mention may be made in particular of polyether triamines, notably of formula, known under the name Jeffamine from the company Hunstman; and notably α,ω-diamino polyethylene glycol and/or polypropylene glycol (with an amine function at the end of the chain) such as the products sold under the names Jeffamine T-403.

As (poly)amine compounds that are triamine compounds, mention may be made in particular of polyether triamines, and notably α,ω-diamino polyethylene glycol and/or polypropylene glycol (with an amine function at the end of the chain) such as the products sold under the names Jeffamine T-403.

According to another particular embodiment of the invention, the (poly)amine compound(s) include more than three primary and/or secondary amine groups, preferably primary amine groups (NH2).

In this variant, the (poly)amine compound(s) are chosen from poly(meth)acrylates or poly(meth)acrylamides bearing lateral primary or secondary amine functions, such as poly(3-aminopropyl)methacrylamide and poly(2-aminoethyl) methacrylate.

Preferably, the (poly)amine compounds are chosen from chitosans (notably poly(D-glucosamine)) and polydimethylsiloxanes comprising primary amine groups at the end of the chain and/or on side chains.

According to this variant, the (poly)amine compound(s) are chosen in particular from poly((C2-C5)alkyleneimines), and preferably polyethylenimines and polypropyleneimines, notably poly(ethyleneimine), in particular the product sold under reference 408700 by the company Aldrich Chemical or under the trade name Lupasol by BASF, notably with a molecular weight of between 1200 and 25 000; poly(allylamine), in particular the product sold under reference 479136 by the company Aldrich Chemical; polyvinylamines and copolymers thereof, notably with vinylamides, in particular vinylamine/vinylformamide copolymers such as those sold under the name Lupamin® 9030 by the company BASF; polyamino acids containing NH₂ groups, such as polylysine, in particular the product sold by the company JNC Corporation (formerly Chisso); amino dextran, in particular the product sold by the company CarboMer Inc; amino polyvinyl alcohol, in particular the product sold by the company CarboMer Inc; acrylamido(C₁-C₆)alkylamine-based copolymers, notably acrylamidopropylamine-based copolymers; and poly(D-glucosamine), for example sold under the reference Kionutrime CSG® by the company Kytozyme.

According to one embodiment, the polydimethylsiloxanes comprising primary amine groups at the end of the chain and/or on side chains are chosen from formula (VII) below:

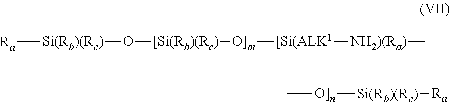

in which formula (VII) Rₐ, which may be identical or different, represents a hydroxyl or (C₁-C₄)alkyl group, R_{b} and R_{c}, which may be identical or different, preferably identical, represent a (C₁-C₄)alkyl group such as methyl, ALK¹ represents a linear or branched (C₁-C₆)alkylene group, optionally interrupted with an N(H) group, m and n are integers greater than or equal to 1; preferably, m and n are such that the weight-average molecular mass of the compound of formula (VII) ranges from 1000 g·mol⁻¹ to 500 000 g·mol⁻¹.

According to an advantageous variant, formula (VII) is such that Rₐ, R_{b} and R_{c} represent a methyl group, ALK1 represents a propylene group, the values of n and m are such that the weight-average molecular weight of the polydimethylsiloxane ranges from 1000 g·mol−1 to 55 000 g·mol−1. As examples of polydimethylsiloxanes of formula (VI), mention may be made of those sold under the names AMS-132, AMS-152, AMS-162, AMS-163, AMS-191 and AMS-1203 by the company Gelest.

According to another variant, formula (VII) is such that Rₐ represents a hydroxyl or (C1-C4)alkyl group, such as methyl, ALK1 represents a (C5-C6)alkylene group substituted with an NH group; preferably, ALK1 represents —(CH2)3-N(H)—(CH2)2-, and m and n are such that the weight-average molecular mass of the compound of formula (VI) ranges from 5000 g·mol−1 to 500 000 g·mol−1.

As amine polymer, mention may also be made of α,ω-diamino polytetrahydrofurans (or polytetramethylene glycol) and α,ω-diamino polybutadienes.

According to a particular embodiment of the invention, the (poly)amine compounds are chosen from hyperbranched polymers comprising at least one amino group and dendrimers bearing at least one amino group, such as PAMAM polyamidoamine dendrimers with an ethylenediamine core and a terminal amine function.

Thus, according to a preferred embodiment, the composition according to the invention comprises a crosslinking agent R chosen from (poly)amine compounds, in particular chosen from chitosans, aminoalkoxysilanes, polydimethylsiloxanes comprising primary amine groups at the end of the chain or on side chains, amodimethicones, polyglucosamines, and mixtures thereof.

More preferentially, the composition according to the invention comprises a crosslinking agent R chosen from spermidine, aminoalkoxysilanes and polydialkylsiloxanes comprising primary amine groups at the end of the chain or on side chains, and even more preferentially chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane and polydimethylsiloxanes comprising terminal aminopropyl groups at the end of the chain, and even more preferentially 3-aminopropyltriethoxysilane (APTES) or oligomers thereof.

### B) (Poly)Thiol Compounds

According to a preferred embodiment, the crosslinking agent R is chosen from (poly)thiol compounds, also known as “(poly)mercapto” compounds.

The (poly)thiol compound may in particular be organic or inorganic, preferably organic.

In a preferred embodiment, the (poly)thiol compound is silicon-based, i.e. it includes one or more thiol groups and it also includes at least one siloxane chain.

In a particular embodiment, the (poly)thiol compound is inorganic. Mention may be made, for example, of polythiol silicones.

The (poly)thiol compound used in a composition according to the invention may in particular be chosen from non-polymeric (poly)thiol compounds.

For the purposes of the present invention, the term “non-polymeric compounds” means compounds which are not directly obtained via a monomer polymerization reaction.

According to one embodiment of the invention, the (poly)thiol compound(s) are organic, non-polymeric and of formula (VIII) below and also the solvates thereof such as hydrates:

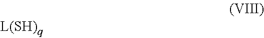

in which formula (VIII):
   q represents an integer greater than or equal to 2; preferably, n is between 2 and 10 and preferably between 2 and 5 inclusive;
   L denotes a saturated or unsaturated linear or branched, or a saturated or unsaturated (hetero)cyclic, multivalent (at least divalent) group, in particular comprising from 1 to 500 carbon and/or silicon atoms, more particularly between 2 and 40 carbon and/or silicon atoms, even more particularly between 3 and 30 carbon and/or silicon atoms, preferably between 6 and 20 carbon atoms;
   L being optionally interrupted and/or terminated with one or more heteroatoms or groups chosen from O, S, N, Si and C(X), and combinations thereof such as —O—, —O—C(X)—, —N(R)—C(X)— or —Si(R_{c})(R_{d})—O— with R representing a hydrogen atom or a (C₁-C₆)alkyl group such as methyl; and/or
   L being optionally substituted with one or more groups chosen from:

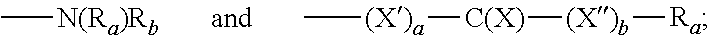

with X, X′ and X″, which may be identical or different, representing an oxygen or sulfur atom, or a group N(R_{b}); a and b being equal to 0 or 1; preferably, the sum of a+b is equal to 1;
Rₐ and R_{b}, which may be identical or different, represent a hydrogen atom or a (C₁-C₆)alkyl or aryl(C₁-C₄)alkyl group, such as benzyl, preferably Rₐ and R_{b} represent a hydrogen atom; and Rₑ and R_{d}, which may be identical or different, represent a (C₁-C₆)alkyl, aryl(C₁-C₄)alkyl or (C₁-C₆)alkoxy group.

According to a particular embodiment of the invention, the (poly)thiol compound(s) are chosen from polythiol compounds, notably polythiol compounds comprising from 2 to 20 carbon atoms.

According to a preferred embodiment, the (poly)thiol compound(s) are non-polymeric and notably of formula (VIII) defined above, in which q is an integer greater than or equal to 2, and preferably p is an integer between 2 and 10 and preferably between 2 and 5 inclusive.

The (poly)thiol compound(s) that are suitable for use in the invention are preferably dithiol compounds.

Preferably, L denotes a C8-C18 multivalent radical, which is notably linear. Preferentially, the liposoluble polythiol is a notably linear C8-C18 dithiol. Advantageously, the C8-C18 chain is a hydrocarbon-based chain, i.e. formed from carbon and hydrogen. In particular, the liposoluble polythiol is a linear C8-C16 and notably C10-C14 dithiol. As (poly)thiol compounds of formula (VII), mention may be made more particularly of 1,8-octanedithiol, 1,10-decanedithiol, 1,12-dodecanedithiol, 1,14-tetradecanedithiol, 1,16-hexadecanedithiol and 1,18-octadecanedithiol. Use is preferably made of 1,10-decanedithiol, 1,12-dodecanedithiol or 1,14-tetradecanedithiol, preferentially 1,12-dodecanedithiol.

According to another particular embodiment of the invention, the (poly)thiol compound(s) are chosen from thiolated alkoxysiloxanes, such as those of formula (VIII′) below: R′₁—Si(OR′₂)z(R′₃)x (VIII′)
in which formula (VIII′):
   R′₁ is a linear or branched, saturated or unsaturated, cyclic or acyclic C₁-C₁₂ hydrocarbon-based chain substituted with one or more groups chosen from the following groups:
      thiol, and
      aryl, aryloxy, arylthio or arylamino, the aryl group being substituted with one or more thiol or thio(C₁-C₆)alkyl groups, preferably thio(C₁-C₆)alkyl, and
      R′₁ is optionally interrupted in its hydrocarbon-based chain with one or more heteroatoms such as O, S, N, a carbonyl group C(O), or a combination thereof such as ester —C(O)—O—, or amide —C(O)—N(H)—, R′₁ being bonded to the silicon atom directly via a carbon atom,
      R′₂ and R′₃, which may be identical or different, represent a linear or branched alkyl group comprising from 1 to 6 carbon atoms, preferably from 1 to 4 carbon atom(s) such as methyl,
      z denotes an integer ranging from 1 to 3, and
      x denotes an integer ranging from 0 to 2, with z+x=3.

Preferably, R′2 represents a linear or branched, preferably linear, alkyl group comprising from 1 to 4 carbon atoms, such as ethyl.

Preferably, R′3 represents a linear or branched, preferably linear, alkyl group comprising from 1 to 4 carbon atoms, such as methyl or ethyl.

Preferably, R′1 is an acyclic chain, in particular R′1 is a linear or branched, saturated or unsaturated, preferably saturated, C1-C6 hydrocarbon-based chain substituted with one or more thiol groups, preferably substituted with one thiol group.

Preferably, R′1 is a saturated linear C1-C6 hydrocarbon-based chain substituted with a thiol group, and R′2 represents an alkyl group comprising from 1 to 4 carbon atoms.

R′3 represents an alkyl group comprising from 1 to 4 carbon atoms. Preferably, z is equal to 3.

According to a more particular embodiment of the invention, the thiolated alkoxysiloxanes are chosen from those of formula (IX) below:

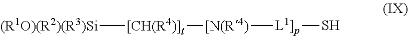

in which formula (IX):
   p is 0 or 1; and t is an integer between 1 and 4, preferably 2;
   R¹ represents a (C₁-C₆)alkyl radical;
   R² and R³, which may be identical or different, preferably identical, are chosen from:
      a (C₁-C₆)alkyl group, in particular a C₁-C₄ alkyl group such as methyl;
      a (C₁-C₆)alkoxy group, in particular a (C₁-C₄)alkoxy group such as methoxy;
   R⁴ and R′⁴, which may be identical or different, represent a hydrogen atom or a (C₁-C₆)alkyl group, such as methyl;
   L¹ represents a divalent, saturated, linear or branched C₁-C₂₀ hydrocarbon-based radical.

According to a particular embodiment of the invention, the thiolated alkoxysiloxanes are chosen from those of formula (IX′) below:

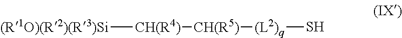

in which formula (IX′):
   q is 0 or 1;
   X represents an oxygen or sulfur atom, preferably a sulfur atom;
   R′¹ denotes a (C₁-C₆)alkyl radical;
   R′² and R′³, which maybe identical or different, preferably identical, are chosen from:
      a (C₁-C₆)alkoxy group, in particular C₁-C₄;
      a (C₁-C₆)alkyl radical;
   R⁵ represents a hydrogen atom or a C₁-C₄ alkyl group optionally substituted with an amino, thiol or hydroxyl group;
   R⁴ represents a hydrogen atom or a C₁-C₄ alkyl group, in particular methyl;
   L² represents a linear or branched, saturated C₁-C₂₀ divalent hydrocarbon-based group, optionally interrupted with a heteroatom such as —N(H)—, and/or optionally substituted with one or more hydroxyl, thiol or amino groups.

Preferably, the thiolated alkoxysilane(s) are chosen from 4-(trimethoxysilyl)-1-butanol, 3-(trimethoxysilyl)-1-propanol, 3-(triethoxysilyl)-1-propanol, 11-(trimethoxysilyl)-1-undecanethiol, 4-(trimethoxysilyl)-2-butanethiol, 2-(triethoxysilyl)ethanethiol, 3-(triethoxysilyl)-1-propanethiol, 2-(trimethoxysilyl)ethanethiol, 3-(trimethoxysilyl)-1-propanethiol and 3-(dimethoxymethylsilyl)-1-propanethiol.

More preferentially, the thiolated alkoxysilane(s) are chosen from 2-(triethoxysilyl)ethanethiol (18236-15-2) and 3-(triethoxysilyl)-1-propanethiol (14814-09-6).

According to a preferred embodiment of the invention, the (poly)thiol compound(s) are chosen from polymeric (poly)thiol compounds.

The polymeric (poly)thiol compounds may be star, comb, brush and dendritic homopolymers or copolymers bearing hydroxyl and/or thiol units. The polymers may be of natural origin such as polysaccharides or polypeptides, or of synthetic origin such as acrylic polymers, polyesters or polyglycols. The thiol units may be present as terminal or side groups.

Examples that may be mentioned include the polymers described in the following scientific articles: Polymers containing groups of biological activity, C. G. Overberger et al., Polytechnic Institute of Brooklyn,
http://pac.iupac.org/publications/pac/pdf/1962/pdf/0402x0521.pdf; EP 1 247 515 A2; U.S. Pat. No. 3,676,440; and EP 1 572 778.

The polymeric (poly)thiol compounds of the invention are preferably organic and/or silicone compounds, more preferentially of formula (X): POLY(SH)_{q} (X)
in which formula (X):
   q is greater than or equal to 2, preferably greater than or equal to 3;
   POLY denotes a polymeric radical which is preferably carbon-based or silicon-based;
POLY being optionally interrupted with one or more heteroatoms or groups chosen from O, S, N, Si and C(X), and combinations thereof such as —O—, —O—C(X)—, —N(R)—C(X)— or —Si(R_{c})(R_{d})—O— with R representing a hydrogen atom or a (C₁-C₆)alkyl group such as methyl; and/or
POLY being optionally substituted with one or more halogen atoms, or a group chosen from Rₐ(R_{b})N— and —(X′)ₐ—C(X)—(X″)_{b}—Rₐ;
   X, X′ and X″, which may be identical or different, represent an oxygen or sulfur atom, or a group N(R_{b});
   a and b being equal to 0 or 1; preferably, the sum a+b is equal to 1;
   Rₐ and R_{b}, which may be identical or different, represent a hydrogen atom or a (C₁-C₁₀)alkyl or aryl(C₁-C₄)alkyl group such as benzyl; preferably, Rₐ and R_{b} represent a hydrogen atom; and
   R_{c} and R_{d}, which may be identical or different, represent a (C₁-C₁₀)alkyl, aryl(C₁-C₄)alkyl or (C₁-C₁₀)alkoxy group.

The methods for preparing the polymeric (poly)thiol compounds used according to the invention are known to those skilled in the art; several methods are reported hereinbelow in a non-limiting manner. The polymeric (poly)thiol compounds used according to the invention may be obtained by polymerization or polycondensation of monomer units bearing thiol or protected thiol functions, optionally as a copolymerization or co-polycondensation of monomer units free of thiol or protected thiol functions.

According to one embodiment of the invention, the polymeric (poly)thiol compounds used according to the invention are polymers which are soluble in cosmetic media, particularly in aqueous or aqueous-alcoholic media. They are more preferentially obtained from amino polymers and the ammonium salts thereof or from polyhydroxylated polymers.

According to another embodiment of the invention, the thiolated polymers used according to the invention are polymers that are soluble in lipophilic media.

According to one embodiment of the invention, the polythiol compound is a polymeric compound of formula (X) in which q denotes an integer greater than or equal to 2, and POLY denotes a carbon-based and/or silicon-based, preferably silicon-based, polymeric radical, POLY also possibly containing one or more heteroatoms chosen from O, N or S, and/or one or more functions chosen from (thio)ester, (thio)ketone, (thio)amide, (thio)urea and (thio)carbamate functions, and/or possibly being substituted with one or more linear or branched (C1-C10)alkyl or linear or branched (C1-C10)alkoxy groups, it being understood that when POLY is substituted, the thiol functions may be borne by the substituent(s).

The weight-average molecular weight of the polythiol polymer compounds, such as those of formula (X), is generally between 500 and 400 000 g·mol−1, preferably between 500 and 150 000 g·mol⁻¹. According to a particular embodiment of the invention, the polythiol compounds are chosen from polyorganosiloxanes including thiol groups on end chains, such as those of formula (XI):

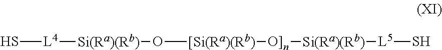

in which formula (XI):
   R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl,
   n represents an integer greater than or equal to 1 and more particularly the value of n is such that the weight-average molecular weight of the silicone ranges from 500 to 55 000 g·mol−1; in particular, n is an integer ranging from 1 to 100, preferably ranging from 5 to 50 and preferentially ranging from 10 to 30, and
   L⁴ and L⁵, which may be identical or different, preferably identical, represent a linear or branched, saturated or unsaturated, optionally cyclic hydrocarbon-based chain comprising from 1 to 100 carbon atoms, optionally interrupted with one or more heteroatoms such as oxygen, sulfur or nitrogen, in particular oxygen, and in particular represent a covalent bond or a (C₁-C₆)alkylene, (C₁-C₆)alkylenoxy, oxy(C₁-C₆)alkylene, (C₁-C₆)alkylenoxy(C₁-C₆)alkylene, (C₁-C₆)alkylenoxy(C₁-C₆)alkylenoxy or oxy(C₁-C₆)alkylenoxy(C₁-C₆)alkylene group, preferably a (C₁-C₆)alkylene, (C₁-C₆)alkylenoxy, oxy(C₁-C₆)alkylene or (C₁-C₆)alkylenoxy(C₁-C₆)alkylene group.

Preferentially, the (poly)thiol compounds are polythiol polyorganosiloxanes, more preferentially polythiol polydimethylsiloxanes, notably chosen from those of formula (XII):

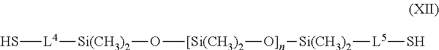

in which formula (XII):
   L⁴ and L⁵ are as defined previously in formula (XI), in particular L⁴ and L⁵ represent a (C₁-C₆)alkylene, (C₁-C₆)alkylenoxy, oxy(C₁-C₆)alkylene or (C₁-C₆)alkylenoxy(C₁-C₆)alkylene group, more preferentially a divalent group chosen from —R₂—, —O—R₂—, —R₂—O— and —R₂—O—R₂—, preferably —R₂—O—R₂—, with R₂ representing a linear or branched, preferably linear, (C₂-C₆)alkylene group, such as ethylene or propylene, preferably n-propylene; and
   n is as defined in formula (XI).

As polythiol compounds of formula (XII), mention may be made of mercaptosiloxanes or thiolated siloxanes in which the thiol functions are at the chain ends, sold by the company Shin-Etsu under the reference X-22-167B, and mercaptosiloxanes in which the mercapto functions are pendent, sold by the company Shin-Etsu under the reference KF-2001, or polydimethylsiloxanes in which the thiol functions are at the chain ends, via thio-n-propyl, 80-120 groups, sold by the company Gelest under the name DMS-SM 21.

Preferentially, the polythiol compounds are polyorganosiloxanes including thiol groups on side chains, such as those of formula (XIII):

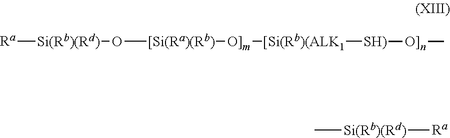

in which formula (XIII):
   R^{a} and R^{b} are as defined in formula (XI) and R^{d} is as defined for R^{a} and R^{b}, preferably R^{a}, R^{b} and R^{d}, which are identical, represent a (C₁-C₆)alkyl group, such as methyl;
   R^{d} may also represent a (C₁-C₆)alkyl group substituted with a (C₁-C₄)alkylamino or amino or thiol group, preferably (C₁-C₄)alkyl such as methyl;
   ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen (in particular O), a (thio)carbonyl group C(X) with X representing O or S, or combinations thereof such as —O—, —O—C(O)— or —C(O)—O—; preferably, ALK₁ represents a (C₁-C₆)alkylene and more preferentially (C₁-C₄)alkylene group such as propylene;
   n and m, which may be identical or different, representing an integer greater than 2 and more particularly the values of m and n are such that the weight-average molecular weight of said polyorganosiloxane is between 1000 and 55 000 g·mol⁻¹;

As examples of polythiol compounds of formula (XIII), mention may be made of those sold by the company Genesee Polymers under the names GP-367, GP-71-SS, GP-800 and GP-710s, preferably GP-367, sold by the company Genesee Polymers.

The polythiol compounds are notably polydimethylsiloxanes including at least two thiol groups, for instance the products SMS-022, SMS-042 and SMS-992 sold by the company Gelest in https://www.gpcsilicones.com/products/silicone-fluids/mercapto-functional, https://www.shinetsusilicone-global.com/products/type/oil/detail/search/deg07.shtml, and 1053_Reactive Silicones_Silanes/Silicones-Gelest.

According to a particular embodiment of the invention, the (poly)thiol compounds are chosen from hyperbranched polymers comprising at least one thiol group and dendrimers bearing at least one thiol group, such as thiolated PAMAM dendrimers.

Preferably, the (poly)thiol compounds used according to the invention are chosen from polydiallylsiloxanes, notably polydimethylsiloxanes, including at least two thiol groups such as those of formula (XIII).

### C) (Poly)Carbonyl Compounds

According to a particular embodiment, the crosslinking agent R is a (poly)carbonyl compound.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde, 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, and nonionic or anionic oxidized polysaccharides such as oxidized inulins, notably those of formula (II) as defined hereinbelow. In particular, the (poly)carbonyl compounds include a saturated or unsaturated, aromatic C5-C7 carbocycle, preferably aromatic, such as phenyl, or non-aromatic and saturated such as cyclohexyl, more preferentially unsaturated and aromatic, such as terephthaldehyde.

According to a particular embodiment, the (poly)carbonyl compound(s) are chosen from nonionic or anionic oxidized polysaccharides comprising one or more aldehyde groups, and optionally one or more anionic groups.

These anionic groups are preferably carboxyl or carboxylate groups.

The nonionic or anionic oxidized polysaccharides according to the invention may be represented by formula (II) below: P—(CHO)ₘ (COOQ)ₙ (II)
in which formula (II):
   P represents a polysaccharide chain preferably consisting of monosaccharides comprising 5 carbon atoms or more than 5 carbon atoms, preferably 6 or more than 6 carbon atoms and more particularly 6 carbon atoms;
   Q is chosen from a hydrogen atom, the ions derived from an alkali metal or an alkaline-earth metal such as sodium or potassium, ammonia, organic amines such as monoethanolamine, diethanolamine, triethanolamine and 3-amino-1,2-propanediol and basic amino acids such as lysine, arginine, sarcosine, ornithine and citrulline;
   m+n is greater than or equal to 1,
   m is such that the degree of substitution of the polysaccharide with one or more aldehyde groups (DS(CHO)) is within the range from 0.001 to 2 and preferably from 0.005 to 1.5;
   n is such that the degree of substitution of the polysaccharide with one or more carboxylic groups (DS(COOX)) is within the range from 0 to 2 and preferably from 0.001 to 1.5.

The expression “degree of substitution DS(CHO) or DS(COOX) of the polysaccharides according to the invention” means the ratio between the number of carbons oxidized to give an aldehyde or carboxylic group for all the repeating units and the number of elementary monosaccharides (even opened by preoxidation) constituting the polysaccharide. The groups CHO and COOX may be obtained during the oxidation of certain carbon atoms, for example on the carbon atoms 2, 3 or 6, of a saccharide unit containing 6 carbon atoms.

Preferably, the oxidation may take place on carbons 2 and 3, more particularly of 0.01% to 75% by number, and preferably of 0.1% to 50% by number of the rings that may have been opened.

The polysaccharide chain, represented by P, is preferably chosen from celluloses, starches, maltodextrins, guar gums, xanthan gums, pullulan gums, agar-agar gums, carrageenan gums, gellan gums, acacia gums, polyxylans and tragacanth gums, and derivatives thereof.

The term “derivative” means the compounds obtained by chemical modification of the mentioned compounds. They may be esters, amides or ethers of said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, pages 1 to 12 (2002).

Another oxidation process is described in the article “Water soluble oxidized starches by peroxide reaction extrusion” Industrial Crops and Products 7, R. E. Wing, J. L. Willet, 45-52 (1997).

According to this embodiment, the (poly)carbonyl compound is associated in its implementation with an amine catalyst as described in the articles Progress in coating 129, 21-25 (2019) and Progress in coating 135, 510-516 (2019); preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

### D) (Poly)Acrylate Compounds

According to a particular embodiment, the crosslinking agent is a (poly)acrylate compound.

The term “(poly)acrylate” means a compound which comprises at least one acrylate ester group H2C═C(Re)—C(O)—Y— with Re representing a hydrogen atom or a (C1-C4)alkyl group, such as methyl, preferably Re representing a hydrogen atom, and Y representing an oxygen atom or an amino group —N(H)—, preferably O.

More particularly, the (poly)acrylate(s) of the invention are of formula (XIV)

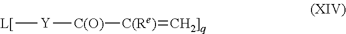

in which formula (XIV) q and L are as defined in formula (VIII); Y and R^{e} are as defined previously, preferably Y═O and R^{e}═H. According to a preferred embodiment, the compounds of formula (XIV) are such that L represents a di- or trivalent, preferably trivalent, hydrocarbon-based chain comprising from 1 to 8 carbon atoms, q is 2 or 3, preferably 3, Y represents O, and Re represents a hydrogen atom.

According to a particular embodiment, the (poly)acrylate compounds are chosen from polyorganosiloxanes including at least one acrylate group on the side chain, such as those of the formula:

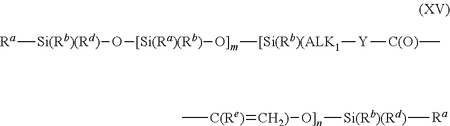

in which formula (XV):
   R^{a}, R^{b} and R^{d} are as defined for formula (XIII), and preferably represent a (C₁-C6)alkyl group such as methyl,
   ALK₁ is as defined for formula (XIII), preferably ALK₁ represents a (C₁-C₆)alkylene group, more preferentially a (C₁-C₄)alkylene group, such as propylene; and
   n and m, which may be identical or different, represent an integer greater than 2 and more particularly the values of m and n are such that the weight-average molecular weight of said polyorganosiloxane is between 1000 and 55 000 g·mol⁻¹.
   Y is as defined previously, preferably O.

More particularly, the (poly)acrylate compound may be chosen from 1,3-butanediol diacrylate, 1,4-butanediol diacrylate, bis(trimethylolpropane) tetraacrylate, glyceryl 1,3-diglycerolate diacrylate, glyceryl propoxylate (1 PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1 PO/OH) diacrylate, pentaerythrityl tetraacrylate, pentaerythrityl triacrylate, poly(propylene glycol) diacrylate, tricyclo[5.2.1.02,6]decanedimethanol diacrylate, trimethylolpropane ethoxylate (1 EO/OH) methyl ether diacrylate, trimethylolpropane propoxylate triacrylate, trimethylolpropane triacrylate, trimethylolpropane trimethacrylate, tri(propylene glycol) diacrylate, trimethylolpropane triacrylate, and tris[2-(acryloyloxy)ethyl]isocyanurate.

The (poly)acrylate compound may also be chosen from N,N′-methylenebisacrylamide.

According to this embodiment, the (poly)acrylate compound is combined in its implementation with an amine catalyst as described, for example, in Progress in coating 129, 21-25 (2019) and Progress in coating 135, 510-516 (2019). Preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

More particularly, the (poly)acrylate compounds are chosen from those of formula (XIV), notably trimethylolpropane triacrylate, and those of formula (XV), notably copolymers of dimethylsiloxane and acryloxypropylmethylsiloxane.

### E) Metal Alkoxides

According to another particular embodiment, the crosslinking agent R is a compound chosen from the metal alkoxides of formulae (XIVₐ), (XIV_{b}), (XIV_{c}) and (XIV_{d}) below and mixtures thereof:

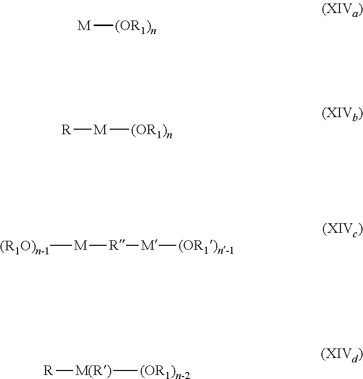

in which formulae (XIVₐ), (XIV_{b}), (XIV_{c}) and (XIV_{d}):
M and M′, which may be identical or different, represent an atom chosen from alkaline-earth metals, transition metals, metals of the lanthanide family, post-transition metals such as aluminium or tin and metalloids such as boron; preferably transition metals such as Ti and post-transition metals such as aluminium;
n and n′ respectively represent the valencies of the atoms represented by M and M′;
R₁ and R₁′, which may be identical or different, represent a linear or branched, saturated or unsaturated hydrocarbon-based group containing from 1 to 30 carbon atoms, preferably from 1 to 6 carbon atoms, said hydrocarbon-based group being optionally interrupted with 1 to 20 heteroatoms chosen from O, N, S and P, notably 0 or N; and/or said hydrocarbon-based group being optionally substituted with one or more hydroxyl or carbonyl groups;
R and R′, which may be identical or different, represent a hydrogen atom or a linear, branched, acyclic or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P, notably 0 or N, and/or said hydrocarbon-based group being optionally substituted with one or more hydroxyl or carbonyl groups;
R″ represents —O—, —N(R2)-, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and P, notably O or N, with R2 representing a linear, cyclic or branched, saturated or unsaturated hydrocarbon-based group containing from 1 to 30 carbon atoms, preferably from 2 to 20 carbon atoms.

Preferably, M and M′, which may be identical or different, represent an atom chosen from transition metals such as titanium or zirconium or alkaline-earth metals such as magnesium, more preferentially chosen from transition metals such as titanium or zirconium, even more preferentially titanium.

Preferably, the organometallic compound(s) are chosen from the alkoxides of formula (XIVa) as defined previously. According to this preferred embodiment, the organometallic compound(s) are more particularly chosen from the alkoxides of formula (XIVa) in which:
M represents an atom chosen from transition metals, metals of the lanthanide family, post-transition metals such as aluminium, tin, metalloids such as boron, or alkaline-earth metals such as magnesium or calcium;
n represents the valency of the atom represented by M;
R₁ represents a linear or branched saturated hydrocarbon-based group containing from 1 to 30 carbon atoms, preferably from 1 to 6 carbon atoms.

According to another more preferred embodiment, the organometallic compound(s) are chosen from the alkoxides of formula (XIVₐ), in which M represents an atom chosen from transition metals, such as zirconium or titanium, metals of the lanthanide family, post-transition metals, such as aluminium or tin, metalloids such as boron, and alkaline-earth metals, such as magnesium, preferably M represents a titanium atom; n represents the valency of the atom represented by M, notably 1, 2, 3 or 4, in particular 4; R₁ represents a methyl, ethyl, 2-ethylhexyl, propyl, isopropyl, n-butyl, isobutyl or t-butyl group. According to an even more preferred embodiment, the organometallic compound(s) are chosen from zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C2H₅)(CH₂)₃CH₃)₄), and mixtures thereof, more preferentially chosen from zirconium propoxide, titanium propoxide, titanium butoxide and mixtures thereof. More preferentially, the crosslinking agent R is a compound of formula (XIVₐ) preferably in which M represents an atom chosen from transition metals, notably titanium such as titanium butoxide.

According to a preferred embodiment, the crosslinking agent is chosen from A) (poly)amine compounds, B) (poly)thiol compounds, C) (poly)carbonyl compounds, D) (poly)acrylate compounds, E) metal alkoxides, and preferably from A) (poly)amine compounds and B) (poly)thiol compounds.

In particular, said (poly)amine compounds A) are chosen from a) chitosans, such as poly(D-glucosamine), b) polyether diamines, particularly α,ω-diamino polyethylene glycols (bearing an amine function at the end of the chain) and 4,7,10-trioxa-1,1-tridecanediamine, c) polyether triamines, such as polyetheramines (or Jeffamine), d) aminoalkoxysilanes, such as APTES, e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH₂) and amodimethicones comprising amine groups on side chains, such as bis-cetearyl amodimethicone, f) polyamine alkylenes such as those of formula (XII′) H₂N-Alk-NH₂, Alk being a (C₁-C₆)alkylene group optionally substituted with one or more amino group(s) and/or optionally interrupted with one or more —N(H)— such as spermidine.

In particular, said (poly)thiol compounds B) are chosen from a) polydialkylsiloxanes bearing thiol functions, and b) alkoxysilanes bearing thiol functions, and in particular are chosen from a) polydialkylsiloxanes bearing thiol functions, preferentially polydimethylsiloxanes comprising thiol groups on the side chain (such as mercaptopropyl), notably those of formula (XIII).

In particular, said (poly)acrylate compounds D) are chosen from those of formula (XIV), notably trimethylolpropane triacrylate, and those of formula (XV), notably copolymers of dimethylsiloxane and acryloxypropylmethylsiloxane, preferably trimethylolpropane triacrylate.

In particular, said metal alkoxide compounds E) are chosen from those of formula (XIVa), such as titanium butoxide.

iii) Cosmetic Active Agent

According to a particular embodiment, the process of the invention uses one or more active agents for caring for keratin fibres.

More particularly, in the process of the invention, according to one embodiment, at least one of the compositions C1, C2, C3, C4 or C5 used comprises one or more active agents for caring for keratin fibres, preferably in a proportion of at least 0.01% by weight relative to the total weight of the composition considered. In particular, the care active agent may be at least one hydrophilic active agent and/or one lipophilic active agent, and preferably a hydrophilic care active agent.

The term “hydrophilic active agent” means a water-soluble or water-dispersible active agent that is capable of forming hydrogen bonds.

According to a particular embodiment of the invention, the process for treating keratin fibres, composition C1, C2, C3, C4 or C5 uses one or more cosmetic active agents. In particular, the cosmetic active agent(s) of the invention are chosen from a) dyestuffs (or colouring agents) chosen from pigments, direct dyes, and mixtures thereof, b) active agents for caring for keratin fibres, preferably the hair, c) UV-screening agents, and d) mixtures thereof.

### UV-Screening Agents

According to one embodiment of the invention, in the process of the invention, at least one of the compositions C1, C2, C3, C4 or C5 used comprises, as cosmetic active agent, at least one UV-screening agent. The UV-screening agent is a UV-screening agent normally used in cosmetics. It may be chosen from the positive list contained in Annex VI of (EC) Regulation No. 1223/2009, which specifies the list of UV-screening agents permitted in cosmetics. The UV-screening agents that are suitable for use in the invention may be of varied nature. They may be lipophilic, hydrophilic or insoluble organic agents.

The term “lipophilic UV-screening agent” means any cosmetic or dermatological screening agent that can be fully dissolved in molecular form in a liquid fatty phase or that can be dissolved in colloidal form (for example in micellar form) in a liquid fatty phase.

The term “hydrophilic UV-screening agent” means any cosmetic or dermatological screening agent that can be fully dissolved in molecular form in a liquid aqueous phase or that can be dissolved in colloidal form (for example in micellar form) in a liquid aqueous phase.

The term “insoluble UV-screening agent” means any cosmetic or dermatological screening agent which is not defined either as a lipophilic UV-screening agent or as a hydrophilic UV-screening agent, and which is in the form of particles in aqueous phase or liquid fatty phase. The UV-screening agents of the composition according to the invention may afford UVA and/or UVB photoprotection.

According to a preferred embodiment, the compositions according to the invention, which are preferably cosmetic compositions, may comprise at least one organic and/or mineral UV-screening agent (for screening out the UV radiation of sunlight).

In particular, the UV-screening agent(s) are chosen from bis-resorcinyl triazine derivatives, dibenzoylmethane derivatives, benzylidenecamphor derivatives, and mixtures thereof. The organic UV-screening agents may also be chosen from anthranilic derivatives; cinnamic derivatives; salicylic derivatives; benzophenone derivatives; phenylbenzotriazole derivatives; benzalmalonate derivatives, notably those mentioned in patent U.S. Pat. No. 5,624,663; phenylbenzimidazole derivatives; imidazolines; 4,4-diarylbutadiene derivatives; bis-benzazolyl derivatives, as described in patents EP 6 693 23 and U.S. Pat. No. 2,463,264; p-aminobenzoic acid (PABA) derivatives; methylenebis(hydroxyphenylbenzotriazole) derivatives, as described in patent applications U.S. Pat. Nos. 5,237,071, 5,166,355, GB 2 303 549, DE 197 26, 184 and EP 893 119; benzoxazole derivatives, such as those described in patent applications EP 0 832 642, EP 1 027 883, EP 1 300 137 and DE 101 62 844; screening polymers and screening silicones such as those notably described in patent application WO 93/04665; α-alkylstyrene-based dimers such as those described in patent application DE 198 55 649; 4,4-diarylbutadienes such as those described in patent applications EP 0 967 200, DE 197 46 654, DE 197 55 649, EP 1 008 586, EP 1 133 980 and EP 133 981; other merocyanine derivatives such as those described in patent applications WO 04/006878, WO 05/058269 and WO 06/032741, and mixtures thereof.

According to a particular embodiment, the concentration of the organic UV-screening agents in the compositions according to the invention ranges from 1% to 50%, preferably from 1% to 40% by weight, and better still, for example, ranges from 5% to 35% by weight, relative to the total weight of the composition.

The UV-screening agent(s) may be mineral UV-screening agents, which are generally pigments. The pigments may or may not be coated.

Thus, the mineral UV-screening agents may be chosen from coated or uncoated pigments, and in particular from coated titanium oxide pigments, silicone-treated titanium oxides, uncoated titanium oxide pigments, uncoated zinc oxide pigments, coated zinc oxide pigments, uncoated cerium oxide pigments, uncoated iron oxide pigments, coated iron oxide pigments, and mixtures thereof.

According to a particular embodiment, compositions C1 to C5 according to the invention are free of mineral UV-screening agents.

According to a particular embodiment, the amount of the mineral UV-screening agent(s) present in the compositions according to the invention may range from 0.01% to 20% by weight relative to the total weight of composition C1 to C5 containing same. It ranges, for example, from 1% to 15% by weight, relative to the total weight of the composition.

According to a particular embodiment, at least one of the compositions C1 to C5 according to the invention also comprises one or more organic UV-screening agents and one or more mineral UV-screening agents.

According to a particular embodiment, the compositions according to the invention comprise a combination of UV-screening agents as described in patent FR 2 977 490, patent application WO 2013/004777 or patent application US 2014/0134120.

According to a particular embodiment, the at least one cosmetic agent is chosen from dyestuffs, preferably chosen from pigments, direct dyes and mixtures thereof, more preferentially pigments.

Needless to say, a person skilled in the art will take care to select this or these optional cosmetic active agent(s), and/or the amount thereof, such that the advantageous properties of the corresponding composition according to the invention are not, or are not substantially, adversely affected by the envisaged addition.

### Dyestuffs

According to a particular embodiment, the process of the invention uses one or more dyestuffs.

More particularly, in the process of the invention, at least one of the compositions C1, C2, C3, C4 or C5 used comprises at least one particulate or non-particulate, water-soluble or water-insoluble dyestuff, preferably in a proportion of at least 0.01% by weight relative to the total weight of the composition considered. For obvious reasons, this amount is liable to vary significantly with regard to the intensity of the desired colour effect and of the colour intensity afforded by the dyestuffs under consideration, and its adjustment clearly falls within the competence of a person skilled in the art.

Preferably, a composition C1, C2, C3, C4 or C5 comprises at least one dyestuff chosen from pigments, direct dyes and mixtures thereof, more preferentially pigments; more preferentially, the pigment(s) of the invention are chosen from carbon black, iron oxides, notably yellow, red and black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides, yellow iron oxides and azo pigments, notably red azo pigments, such as SunPuro Red Iron Oxide.

### Pigments

For the purposes of the invention, the term “pigment” means any compound that is capable of imparting colour to keratin fibres. These compounds have a solubility in water at 25° C. and at atmospheric pressure (760 mmHg) of less than 0.05% by weight, and preferably less than 0.01% by weight.

As pigments that are suitable for use in the invention, mention may notably be made of the organic and/or mineral pigments known in the art, notably those described in Kirk-Othmer's Encyclopedia of Chemical Technology and in Ullmann's Encyclopedia of Industrial Chemistry. These pigments may be synthetic or natural. These pigments may be in pigment powder or paste form. They may be coated or uncoated. These pigments may be chosen, for example, from mineral pigments, organic pigments, lakes, pigments with special effects such as nacres or glitter flakes, and mixtures thereof.

A pigment that is suitable for use in the invention may be chosen from mineral pigments.

The term “mineral pigment” means any pigment that satisfies the definition in Ullmann's encyclopedia in the chapter on inorganic pigments. Among the mineral pigments that are useful in the present invention, mention may be made of manganese violet, ultramarine blue, chromium hydrate, ferric blue and titanium, zirconium or cerium oxides or dioxides, and also of zinc, iron or chromium oxides.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30. They may also be pigments having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Advantageously, the pigments may be iron oxides and/or titanium dioxides.

A pigment that is suitable for use in the invention may be chosen from organic pigments.

The term “organic pigment” refers to any pigment that satisfies the definition in Ullmann's encyclopaedia in the chapter on organic pigments. Among the organic pigments that are useful in the present invention, mention may be made of nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds. In particular, the white or coloured organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525,15580,15620,15630,15800,15850,15865, 15880,26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names: Cosmenyl Yellow IOG: Yellow 3 pigment (CI 11710); Cosmenyl G yellow: Yellow 1 pigment (CI 11680); Cosmenyl GR orange: Orange 43 pigment (CI 71105); Cosmenyl R red: Red 4 pigment (CI 12085); Cosmenyl FB carmine: Red 5 pigment (CI 12490); Cosmenyl RL violet: Violet 23 pigment (CI 51319); Cosmenyl A2R blue: Blue 15.1 pigment (CI 74160); Cosmenyl GG green: Green 7 pigment (CI 74260); Cosmenyl R black: Black 7 pigment (CI 77266).

The pigments in accordance with the invention may also be in the form of composite pigments, as described in patent EP 1 184 426. These composite pigments may particularly be composed of particles including an inorganic core, at least one binder for attaching the organic pigments to the core, and at least one organic pigment which at least partially covers the core.

The organic pigment may also be a lake.

The term “lake” refers to dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium. Among the dyes adsorbed on organic substrates, mention may be made of carminic acid. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985), D & C Green 5 (CI 61 570), D & C Yellow 10 (CI 77 002), D & C Green 3 (CI 42 053), D & C Blue 1 (CI 42 090), FDC Red 4, D & C Red 6, D & C Red 22, D & C Red 28, D & C Red 30, D & C Orange 4, D & C Yellow 8, D & C Green 5, D & C Red 17, D & C Green 6, D & C Yellow 11, D & C Violet 2, Sudan red, carotenes (p-carotene, lycopene), xanthophylls (capsanthin, capsorubin, lutein), palm oil, Sudan brown, quinoline yellow, annatto, curcumin, betanin (beet), carmine, copper chlorophyllin, methylene blue, anthocyanins (enocianine, black carrot, hibiscus, elderberry), riboflavin, beet juice and caramel.

An example of a lake that may be mentioned is the product known under the following name: D&C Red 7 (CI 15 850:1).

The pigment may also be a pigment with special effects.

The term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof. As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C. The pigments used in the composition may be surface-treated with an organic agent. According to a particular embodiment, the dispersant(s) are of amino silicone type other than the alkoxysilanes described previously and are cationic. Preferably, the pigment(s) is (are) chosen from mineral, mixed mineral-organic, or organic pigments.

According to a particular embodiment, the pigment(s) according to the invention are organic pigments, preferentially organic pigments surface-treated with an organic agent chosen from silicone compounds.

According to another embodiment of the invention, the pigment(s) according to the invention are mineral pigments.

According to a particular embodiment of the invention, the cosmetic active agent is a dyestuff chosen from one or more direct dyes.

The term “direct dye” means natural and/or synthetic dyes, other than oxidation dyes. These are dyes which will spread superficially over the fibre. They may be ionic or nonionic, preferably cationic or nonionic.

Among the direct dyes that are suitable for use in the invention, mention may be made of azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (A) and (B) below and the azo cationic dyes of formulae (C) and (D) below:

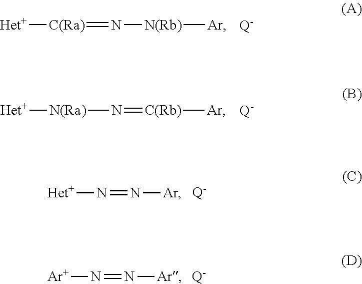

in which formulae (A) to (D):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as optionally substituted (C₁-C₈)alkyl, optionally substituted (C₁-C₈)alkoxy, (di)(C₁-C₈)(alkyl)amino optionally substituted on the alkyl group(s) with a hydroxyl group, aryl(C₁-C₈)alkylamino, and optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group; or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (A) to (D) as defined previously, more particularly the cationic direct dyes bearing an endocyclic cationic charge described in patent applications WO 95/15144, WO 95/01772 and EP 714 954, preferentially the direct dyes of formulae (E) and (F) below:

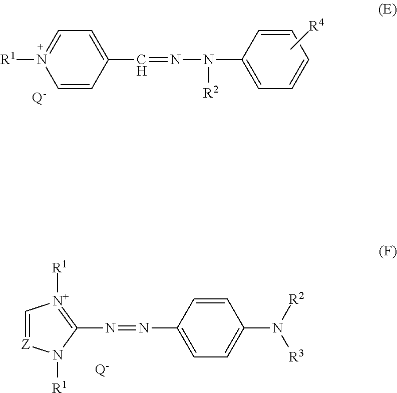

in which formulae (E) and (F):
R¹ represents a (C₁-C₄)alkyl group such as methyl;
R² and R³, which may be identical or different, represent a hydrogen atom or a (C₁-C₄)alkyl group, such as methyl;
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
Z represents a CH group or a nitrogen atom, preferentially CH;
Q⁻ is an anionic counterion as defined previously, in particular a halide, such as chloride, or an alkyl sulfate, such as methyl sulfate or mesyl.

In particular, the dyes of formulae (E) and (F) are chosen from Basic Red 51, Basic Yellow 87 and Basic Orange 31 or derivatives thereof with Q− being an anionic counterion as defined previously, particularly a halide such as chloride, or an alkyl sulfate such as methyl sulfate or mesyl. The direct dyes may be chosen from anionic direct dyes. The anionic direct dyes of the invention are dyes commonly referred to as “acid” direct dyes owing to their affinity for alkaline substances.

The term “anionic direct dye” means any direct dye including in its structure at least one CO₂R′ or SO₃R′ substituent with R′ denoting a hydrogen atom or a cation originating from a metal or an amine, or an ammonium ion.

The anionic direct dyes may be chosen from direct nitro acid dyes, azo acid dyes, azine acid dyes, triarylmethane acid dyes, indoamine acid dyes, anthraquinone acid dyes, indigoid dyes and natural acid dyes.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and particularly henna-based poultices or extracts.

Preferably, the direct dyes are chosen from anionic direct dyes.

The dyestuffs, preferably the pigments, may be present in concentrations ranging from 0.01% to 30% by weight, preferably from 0.2% to 20% by weight and more particularly from 0.5% to 10% relative to the total weight of the composition containing them.

The direct dye(s) may be present in concentrations ranging from 0.001% to 10% by weight relative to the total weight of the composition, preferably from 0.005% to 5% by weight relative to the total weight of the composition containing them.

Preferably, the cosmetic active agent(s), in particular the dyestuff(s) and more particularly the pigment(s), are introduced into compositions C1, C2, C3, C4 or C5.

### Care Active Agents

According to one embodiment of the invention, in the process of the invention, compositions C1, C2, C3, C4 or C5 comprise one or more care active agents, preferably in an amount of at least 0.01% by weight relative to the total weight of the composition considered. In particular, the care active agent may be at least one hydrophilic active agent and/or one lipophilic active agent, and preferably a hydrophilic care active agent.

The term “hydrophilic active agent” means a water-soluble or water-dispersible active agent that is capable of forming hydrogen bonds.

The cosmetic active agent(s) for caring for keratin fibres, notably the hair, may notably be chosen from: a1) vitamins and derivatives thereof; b1) humectants; c1) antioxidant compounds; and mixtures thereof.

The care active agent(s) may in particular be present, in the composition containing same, in a content ranging from 0.01% to 30% by weight, relative to the weight of the composition, and preferably from 0.02% to 25% by weight.

Advantageously, the process for treating keratin fibres is a makeup process, notably for the hair, in particular a process for caring for, styling and/or colouring keratin fibres, and preferably the hair.

### iv) Fatty Phase—Fatty Substances

According to a particular embodiment, the process of the invention uses one or more fatty substances, in particular one or more oils, preferably volatile oils.

More particularly, in the process of the invention, at least one of the compositions C1, C2, C3, C4 or C5 used in the process of the invention comprises a fatty phase.

In particular, at least one of the compositions C1, C2, C3, C4 or C5 used in the process of the invention comprises one or more fatty substances, in particular one or more oils, preferably volatile oils.

The term “oil” refers to a fatty substance that is liquid at room temperature (20° C.) and atmospheric pressure (760 mmHg).

The term “hydrocarbon-based oil” means an oil formed essentially from, or even constituted of, carbon and hydrogen atoms, and possibly oxygen and nitrogen atoms, and not containing any silicon or fluorine atoms. It may contain alcohol, ester, ether, carboxylic acid, amine and/or amide groups.

According to one embodiment of the invention, the oil(s) are chosen from volatile oils, in particular:
hydrocarbon-based oils containing from 8 to 16 carbon atoms, and notably:
   branched C₈-C₁₆ alkanes, for instance isoalkanes such as iso-alkanes (also known as isoparaffins) such as C₁₃-C₁₆ isoparaffin, isododecane, isodecane, isohexadecane, and for example the oils sold under the Isopar or Permethyl trade names, alone or as mixtures, preferably isododecane (also known as 2,2,4,4,6-pentamethylheptane), more preferentially isododecane;
   linear alkanes, for example C₁₁-C₁₆ alkanes, alone or as mixtures, for instance hexane, decane, undecane, tridecane, or n-dodecane (C12) and n-tetradecane (C14), undecane-tridecane mixtures, mixtures of n-undecane (C11) and n-tridecane (C13), and mixtures thereof and also mixtures of n-undecane (C11) and n-tridecane (C13);
   volatile, non-aromatic cyclic C₅-C₁₂ alkanes;
short-chain esters containing from 3 to 8 carbon atoms in total, such as ethyl acetate, methyl acetate, propyl acetate or n-butyl acetate;
carbonate hydrocarbon-based oils of structure R′₁—O—C(O)—O—R′₂ in which R′₁ and R′₂ independently denote a linear, branched or cyclic C₄-C₈ alkyl group, preferably a C₄-C₈ alkyl group, advantageously chosen from dibutyl carbonate or dipentyl carbonate;
ether oils of formula R₁—O—R₂ in which R₁ and R₂ independently denote a linear, branched or cyclic C₄-C₈ alkyl group, preferably a C₄-C₈ alkyl group;
silicone oils comprising in particular from 2 to 7 silicon atoms, these silicone oils optionally including alkyl or alkoxy groups containing from 1 to 10 carbon atoms, such as dimethicones of viscosity 5 and 6 cSt, cyclopentadimethylsiloxane, dodecamethylpentasiloxane, cyclohexadimethylsiloxane, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof;
more preferentially, the volatile oil(s) b) are chosen from C₈-C₁₆ alkanes, notably branched alkanes such as isododecane.

In particular, at least one of the compositions C1, C2, C3, C4 or C5 used in the process according to the invention comprises one or more non-volatile oils, notably chosen from:
non-volatile fluoro oils, notably chosen from fluorinated polyethers and also from fluorosilicone oils, fluorosilicones;
non-volatile silicone oils, notably chosen from non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxy phenyl trimethicone; and also mixtures thereof;
non-volatile apolar hydrocarbon-based oils notably chosen from linear or branched compounds of mineral or synthetic origin: i) liquid paraffin, ii) squalane, isoeicosane, iii) mixtures of linear, saturated hydrocarbons, more particularly C₁₅-C₂₈ hydrocarbons, such as mixtures whose INCI names are (C₁₅-C₁₅)alkane, (C₁₅-C₂₁)alkane, (C₂₁-C₂₈)alkane, iv) hydrogenated or non-hydrogenated polybutenes; v) hydrogenated or non-hydrogenated polyisobutenes, preferably hydrogenated, vi) hydrogenated or non-hydrogenated polydecenes, vii) decene/butene copolymers, butene/isobutene copolymers and viii) mixtures thereof;
non-volatile polar hydrocarbon-based oils, which may be chosen from:
i) saturated, unsaturated, linear or branched C₁₀-C₂₆ fatty alcohols, preferably monoalcohols; advantageously, the C₁₀-C₂₆ alcohols are fatty alcohols, which are preferably branched when they comprise at least 16 carbon atoms; preferably, the fatty alcohol comprises from 10 to 24 carbon atoms, and more preferentially from 12 to 22 carbon atoms, notably such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C₄ to C₃₆, and notably from C₁₈ to C₃₆, these oils possibly being linear or branched, and saturated or unsaturated; by way of example, mention may notably be made of heptanoic or octanoic triglycerides, caprylic/capric acid triglycerides, plant oils such as wheatgerm oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon-based esters of formula R—C(O)—OR′ in which R—C(O)—O— represents the carboxylic acid residue containing from 2 to 40 carbon atoms, and R′ represents a hydrocarbon-based chain containing from 1 to 40 carbon atoms, aliphatic hydrocarbon-based esters of alkylene glycol, in particular ethylene glycol or propylene glycol; the total number of carbon atoms being advantageously at least 10; notably chosen from isoamyl laurate, cetostearyl octanoate, isopropyl myristate, isopropyl palmitate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate or tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate;
iv) hydroxylated esters such as polyglyceryl-2 triisostearate;
v) aromatic esters such as tridecyl trimellitate, C₁₂-C₁₅ alcohol benzoate, the 2-phenylethyl ester of benzoic acid, and butyloctyl salicylate;
vi) linear fatty acid esters with a total carbon number ranging from 35 to 70, for instance pentaerythrityl tetrapelargonate;
vii) esters of C₂₄-C₂₈ branched fatty acids or fatty alcohols such as triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetrakis(2-decyltetradecanoate);
viii) the polyesters obtained by condensation of dimer and/or trimer of unsaturated fatty acid and of diol, such as those with the INCI name Dilinoleic Acid/Butanediol Copolymer or Dilinoleic Acid/Propanediol Copolymer; the polyesters obtained by condensation of fatty acid dimer and of diol dimer, such as dimer dilinoleyl dimer dilinoleate;
ix) synthetic ethers containing from 10 to 40 carbon atoms, such as dicaprylyl ether;
x) dialkyl carbonates, the two alkyl chains possibly being identical or different, such as dicaprylyl carbonate;
xi) vinylpyrrolidone copolymers such as vinylpyrrolidone/1-hexadecene copolymer; and
xii) mixtures thereof;
the non-volatile carbonate oils may be chosen from the carbonates of formula R₈—O—C(O)—O—R₉, with R₈ and R₉, which may be identical or different, representing a linear or branched C₄ to C₁₂ and preferentially C₆ to C₁₀ alkyl chain; the carbonate oils may be dicaprylyl carbonate (or dioctyl carbonate), bis(2-ethylhexyl) carbonate, dipropylheptyl carbonate, dibutyl carbonate, dineopentyl carbonate, dipentyl carbonate, dineoheptyl carbonate, diheptyl carbonate, diisononyl carbonate or dinonyl carbonate and preferably dioctyl carbonate;
oils known as non-volatile ether oils of formula R₁—O—R₂ in which R₁ and R₂ independently denote a linear, branched or cyclic C₆-C₂₄ alkyl group, preferably a C₆-C₁₈ alkyl group, and preferably C₈-C₁₂ alkyl group. It may be preferable for R₁ and R₂ to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethyl butyl ether, cetyl dimethyl butyl ether, cetyl isobutyl ether and mixtures thereof;
more preferentially c) the non-volatile oil(s) are chosen from hydrogenated or non-hydrogenated polyisobutenes, preferably hydrogenated, for instance the non-volatile compounds of the Parleam® range; mixtures of C₁₅-C₁₉ alkanes, and from linear aliphatic hydrocarbon-based esters of formula R—C(O)—OR′ in which R—C(O)—O represents a carboxylic acid residue containing from 2 to 40 carbon atoms, and R′ represents a hydrocarbon-based chain containing from 1 to 40 carbon atoms, as defined previously, notably isononyl isononanoate.

Very preferentially, the process of the invention uses one or more hydrocarbon-based oils, preferably isododecane.

In particular containing from 8 to 16 carbon atoms, and notably branched C8-C16 alkanes, for instance isoalkanes such as C13-C16 isoparaffins, isododecane, isodecane, isohexadecane, alone or as mixtures, the amount of oil(s) in at least one of the compositions C1, C2, C3, C4 or C5 used in the process according to the invention is between 1% and 99% by weight, relative to the total weight of the composition, more particularly between 10% and 90% by weight, preferentially between 25% and 75%, better still between 30% and 70% by weight, relative to the total weight of the composition.

### The Compositions:

According to one embodiment, the process for treating keratin fibres and the composition(s) C1 to C5 used in the process of the invention comprise water.

According to an advantageous variant, C1 is aqueous or aqueous-alcoholic.

According to an advantageous variant, C2 is aqueous or aqueous-alcoholic.

According to an advantageous variant, C3 is aqueous or aqueous-alcoholic.

According to an advantageous variant, C4 is aqueous or aqueous-alcoholic.

According to an advantageous variant, C5 is aqueous or aqueous-alcoholic.

According to another particular embodiment of the invention, C1 is an anhydrous composition. According to another variant, C2 is anhydrous. According to another variant, C3 is anhydrous. According to yet another variant, C4 is anhydrous. According to another variant, C5 is anhydrous. in particular, C1 and/or C4 are anhydrous. In particular, C4 is anhydrous, and it comprises at least one oil, notably a volatile oil such as isododecane.

The compositions C1 to C5 used in the process of the invention may also comprise one or more organic solvents.

The term “organic solvent” means an organic substance that is capable of dissolving another substance without chemically modifying it.

Examples of organic solvents that may be mentioned include a) C₂-C₆ alkanols, such as ethanol and isopropanol; b) polyols that are miscible with water at room temperature (25° C.), notably chosen from polyols notably containing from 2 to 10 carbon atoms, preferably containing from 2 to 6 carbon atoms, such as glycerol, propylene glycol, 1,3-propanediol, butylene glycol, pentylene glycol, hexylene glycol, dipropylene glycol, diethylene glycol or diglycerol; c) polyol ethers, such as 2-butoxyethanol, propylene glycol monomethyl ether, diethylene glycol monoethyl ether or diethylene glycol monomethyl ether; and also d) aromatic alcohols, such as benzyl alcohol or phenoxyethanol, and mixtures thereof.

According to a particular embodiment, the composition also comprises one or more polyols notably chosen from polyols notably containing from 2 to 10 carbon atoms, preferably containing from 2 to 6 carbon atoms, such as glycerol.

In particular, the compositions C1, C2, C3, C4 or C5 (preferably C2 or C3) are aqueous or aqueous-alcoholic (ethanol/water mixture in particular in a volume ratio of between 1/99 to 99/1, more particularly between 10/90 to 90/10, even more particularly between 20/80 and 80/20, preferably 40/60 to 60/40 such as 50/50).

Compositions C1, C2, C3, C4 or C5 may be in anhydrous, water-in-oil emulsion or oil-in-water emulsion form.

### The Adjuvants

Compositions C1, C2, C3, C4 or C5 used in the process of the invention may also comprise one or more adjuvants chosen from fragrances, preserving agents, fillers, free-radical scavengers, polymers, thickeners or film-forming agents, surfactants, trace elements, softeners, sequestrants and propellants.

Another subject of the invention is a composition C1, which is preferably aqueous or aqueous-alcoholic, which comprises i) at least one compound of formula (I) or (Ia) and iii) optionally at least one active agent as defined previously, preferably at least one dyestuff, such as at least one pigment as defined previously; and optionally iv) one or more fatty substances as defined previously.

Another subject of the invention is a composition C2, which is preferably aqueous or aqueous-alcoholic, which comprises i) at least one compound of formula (I) or (Ia) as defined previously, ii) at least one crosslinking agent as defined previously and iii) optionally at least one active agent as defined previously; and optionally iv) one or more fatty substances as defined previously.

Another subject of the invention is a composition C3, which is preferably aqueous or aqueous-alcoholic, which comprises i) at least one compound of formula (I) or (Ia) as defined previously, ii) at least one crosslinking agent as defined previously and iii) at least one active agent as defined previously; and optionally iv) one or more fatty substances as defined previously.

Another subject of the invention is the use of at least one ingredient i) combined with at least one ingredient ii) and optionally combined with at least one ingredient iii) and optionally combined with at least one ingredient iv), said ingredients i) to iv) being as defined previously, for treating keratin fibres, in particular for caring for, styling, holding the shape (curls) of and/or colouring keratin fibres, in particular the hair.

Another subject of the invention is the cosmetic use of composition C3 as defined previously, for treating keratin fibres, in particular for colouring keratin fibres, notably the hair.

Another subject of the invention is the cosmetic use of composition C2 as defined previously, for treating keratin fibres, in particular for styling keratin fibres, preferably the hair.

The invention is illustrated in greater detail in the examples that follow. The amounts are indicated as weight percentages.

### EXAMPLES

### Compound 1: K-FLEX 7301 Sold by King Industrie

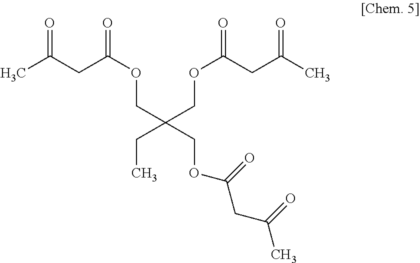 [SMILES: CCC(COC(=O)CC(C)=O)(COC(=O)CC(C)=O)COC(=O)CC(C)=O]

### Compound 2: Glucose Bearing Acetoacetate Functions (5 eq)

Compound 2 was synthesized according to the following scheme:

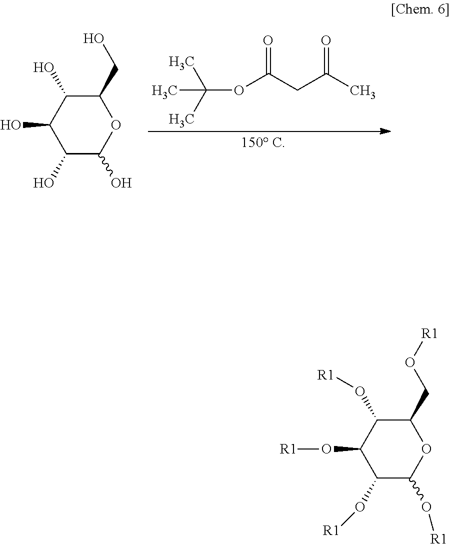

in which scheme R1, which may be identical or different, represents a hydrogen atom or a-methylcarbonylmethylcarbonyl group —C(O)—CH2-C(O)—CH3, it being understood that at least two radicals R1 represent —C(O)—CH2-C(O)—CH3.

100 g of D-glucose and 482.90 g of tert-butyl acetoacetate are placed in a 1-litre three-necked flask equipped with a mechanical stirrer and a distillation column. The reaction medium is heated using an oil bath (oil bath temperature 150° C.-160° C.) for 5 h. After 5 h, NMR confirmed the grafting of the acetoacetate function. The reaction medium is then concentrated on a rotavapor at 150° C. under continuous vacuum. A highly viscous dark brown liquid is obtained, corresponding after analysis to the expected product.

### Compound 3: Mannose Bearing Acetoacetate Functions (5 eq)

Compound 3 was synthesized according to the following scheme:

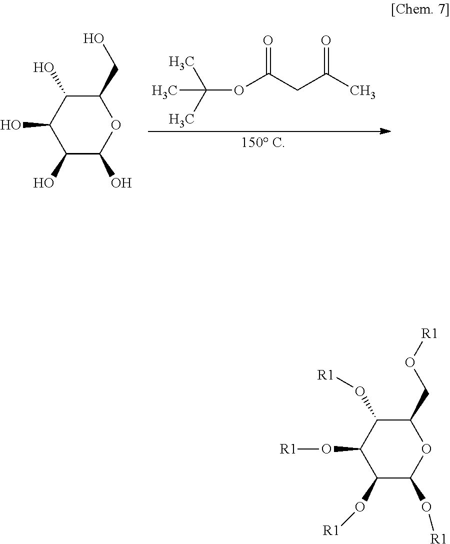

in which scheme R1, which may be identical or different, represents a hydrogen atom or a —C(O)—CH₂—C(O)—CH₃ group, it being understood that at least two radicals R1 represent —C(O)—CH₂—C(O)—CH₃.

100 g of D-mannose and 482.90 g of tert-butyl acetoacetate are placed in a 1-litre three-necked flask equipped with a mechanical stirrer and a distillation column. The reaction medium is heated using an oil bath (oil bath temperature 140° C.-150° C.) for 5 h. After 5 h, NMR confirmed the grafting of the acetoacetate function. The reaction medium is then concentrated on a rotavapor at 150° C. under continuous vacuum. A viscous yellow liquid is obtained, corresponding after analysis to the expected product.

### Compound 4: Xylitol Bearing Acetoacetate Functions (5 eq)

Compound 4 was synthesized according to the following scheme:

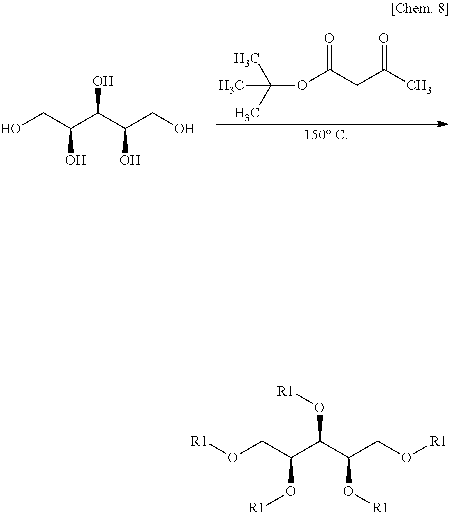

in which scheme R1, which may be identical or different, represents a hydrogen atom or a —C(O)—CH2-C(O)—CH3 group, it being understood that at least two radicals R1 represent —C(O)—CH2-C(O)—CH3.

25 g of xylitol and 142.69 g of tert-butyl acetoacetate are placed in a 500 ml three-necked flask equipped with a mechanical stirrer and a distillation column. The reaction medium is heated using an oil bath (oil bath temperature 140° C.-150° C.) for 5 h. After 5 h, NMR confirmed the grafting of the acetoacetate function. The reaction medium is then concentrated on a rotavapor at 150° C. under continuous vacuum. A highly viscous brown liquid is obtained, corresponding after analysis to the expected product.

### Compound 5: Sorbitol Bearing Acetoacetate Functions (6 eq)

Compound 5 was synthesized according to the following scheme:

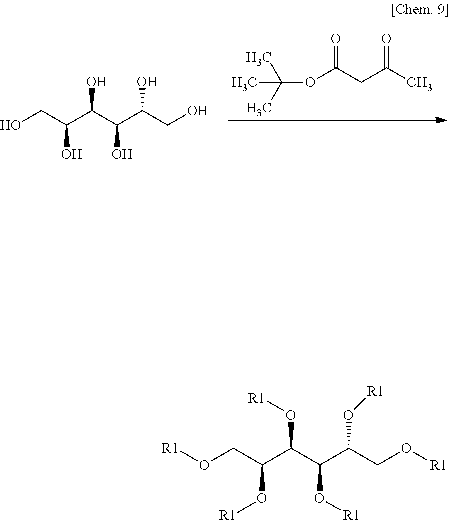

in which scheme R1, which may be identical or different, represents a hydrogen atom or a —C(O)—CH₂—C(O)—CH₃ group, it being understood that at least two radicals R1 represent —C(O)—CH₂—C(O)—.

100 g of sorbitol and 573.20 g of tert-butyl acetoacetate are placed in a 1-litre three-necked flask equipped with a mechanical stirrer and a distillation column. The reaction medium is heated using an oil bath (oil bath temperature 140° C.-150° C.) for 5 h. After 5 h, NMR confirmed the grafting of the acetoacetate function. The reaction medium is then concentrated on a rotavapor at 150° C. under continuous vacuum. A viscous yellow-orange liquid is obtained, corresponding after analysis to the expected product.

### Compound 6: Sorbitol Bearing Acetoacetate Functions (3 eq)

Compound 6 was synthesized according to the following scheme:

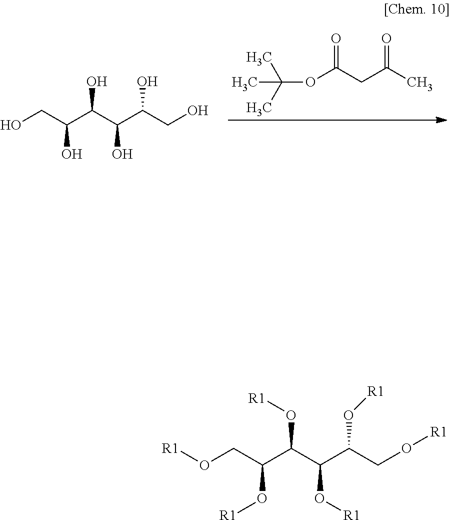

in which scheme R1, which may be identical or different, represents a hydrogen atom or a —C(O)—CH₂—C(O)—CH₃ group, it being understood that at least two radicals R1 represent —C(O)—CH2-C(O)—CH3.

100 g of sorbitol and 260.54 g of tert-butyl acetoacetate are placed in a 1-litre three-necked flask equipped with a mechanical stirrer and a distillation column. The reaction medium is heated using an oil bath (oil bath temperature 140° C.-150° C.) for 5 h. After 5 h, NMR confirmed the grafting of the acetoacetate function. The reaction medium is then concentrated on a rotavapor at 150° C. under continuous vacuum. A viscous yellow liquid is obtained, corresponding after analysis to the expected product.

### Compound 7: Dipentaerythritol Bearing Acetoacetate Functions (6 eq)

Compound 7 was synthesized according to the following scheme:

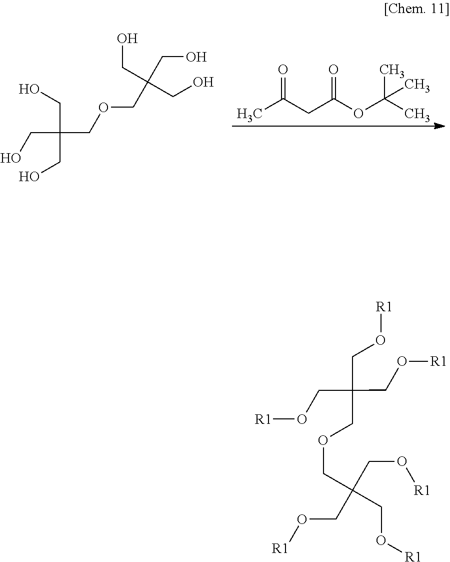

in which scheme R1, which may be identical or different, represents a hydrogen atom or a —C(O)—CH₂—C(O)—CH₃ group, it being understood that at least two radicals R1 represent —C(O)—CH2-C(O)—CH3.

50 g of dipentaerythritol and 195.33 g of tert-butyl acetoacetate are placed in a 1-litre three-necked flask equipped with a mechanical stirrer and a distillation column. The reaction medium is heated using an oil bath (oil bath temperature 140° C.-150° C.) for 4 h. After 4 h, NMR confirms the grafting of the acetoacetate function. The reaction medium is then concentrated on a rotavapor at 150° C. under continuous vacuum. A viscous orange-brown liquid is obtained, corresponding after analysis to the expected product.

### Compound 8: Pentaerythrityl Tetraacetoacetate

Compound 8 was synthesized according to the following scheme:

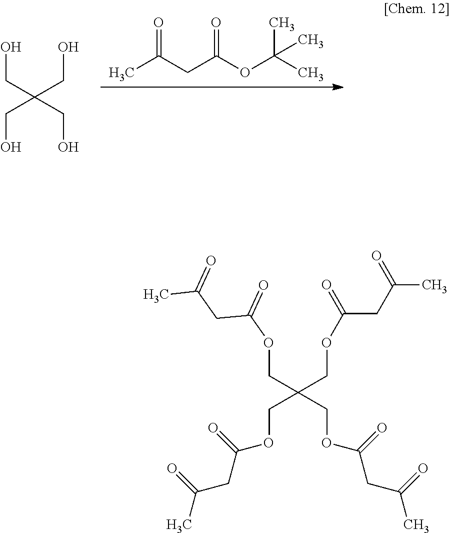 [SMILES: OCC(CO)(CO)CO ; CC(=O)CC(=O)OC(C)(C)C ; CC(=O)CC(=O)OCC(COC(=O)CC(C)=O)(COC(=O)CC(C)=O)COC(=O)CC(C)=O]

100 g of pentaerythritol and 510.89 g of tert-butyl acetoacetate are placed in a 1-litre three-necked flask equipped with a mechanical stirrer and a distillation column. The reaction medium is heated using an oil bath (oil bath temperature 140° C.-150° C.) for 4 h.

After 4 h, NMR confirms the grafting of the acetoacetate function. The reaction medium is then concentrated on a rotavapor at 150° C. under continuous vacuum. A clear viscous yellow liquid is obtained, corresponding after analysis to the expected product.

### Compound 9: Sucrose Bearing Acetoacetate Functions (8 eq)

Compound 9 was synthesized according to the following scheme:

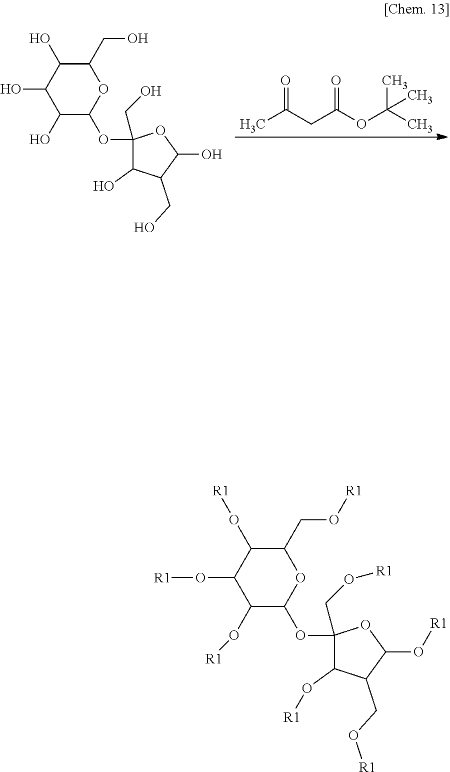

in which scheme R1, which may be identical or different, represents a hydrogen atom or a —C(O)—CH₂—C(O)—CH₃ group, it being understood that at least two radicals R1 represent —C(O)—CH₂—C(O)—CH₃.

100 g of sucrose and 406.55 g of tert-butyl acetoacetate are placed in a 1-litre three-necked flask equipped with a mechanical stirrer and a distillation column. The reaction medium is heated using an oil bath (oil bath temperature 140° C.-150° C.) for 4 h. After 4 h, NMR confirms the grafting of the acetoacetate function. The reaction medium is then concentrated on a rotavapor at 150° C. under continuous vacuum. A viscous yellow-orange liquid is obtained, corresponding after analysis to the expected product.

Amino crosslinking agent 1
Spermidine
CAS Number: 124-20-9
Supplier: Sigma-Aldrich

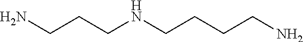

Amino crosslinking agent 2
(3-Aminopropyl)triethoxysilane
(APTES) CAS No.: 919-30-2
Supplier: Sigma-Aldrich

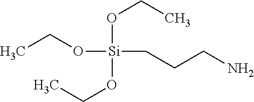

Amino crosslinking agent 3
4,7,10-Trioxa-1,1-tridecanediamine
CAS Number: 4246-51-9
Supplier: Sigma-Aldrich
51-9

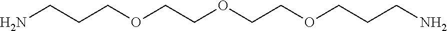 [SMILES: NCCCOCCOCCOCCCN]

Amino crosslinking agent 4
Bis-cetearyl amodimethicone
Supplier: Momentive Performance
Materials

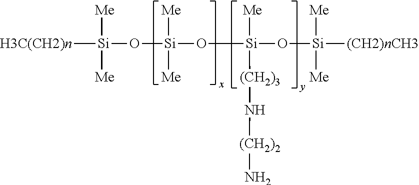

Thiol crosslinking agent 5
[4-6% (mercaptopropyl)methylsiloxane-
dimethylsiloxane copolymer
(CAS No.: 102783-03-9)
Supplier: Gelest
Molecular weight: 6000-8000

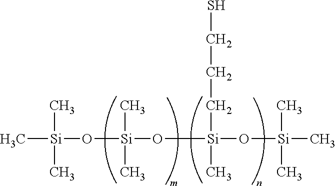

Polyacrylate crosslinking agent 6
Trimethylolpropane triacrylate (CAS
No.: 15625-89-5)
Supplier: Sigma-Aldrich

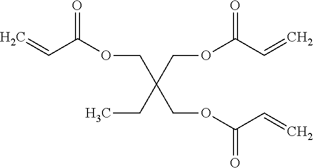 [SMILES: C=CC(=O)OCC(CC)(COC(=O)C=C)COC(=O)C=C]

Metal alkoxide crosslinking agent 7
Titanium butoxide
CAS number: 5593-70-4
Supplier: Sigma-Aldrich

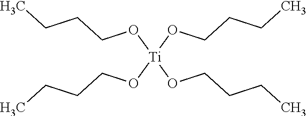

Amino crosslinking agent 8
Vegetable chitosan
Poly(D-glucosamine)
Commercial reference: Kiosmetine
CSG

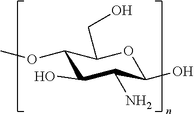

Amino crosslinking agent 9
Jeffamine T403
Polyetheramine (Mw = 440 g/mol)
AHEW (81 g/eq)

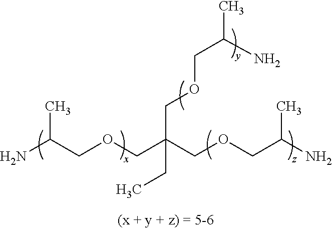

### Implementation of the Invention

### One-Gesture Application:

Compounds 1 to 9 bearing acetoacetate functions and crosslinking agents 1 to 9 are mixed together before application to the hair. The system remains fluid long enough to allow application to the hair. The new material generated on the hair makes it possible, after evaporation of the volatile solvents, to obtain transfer-resistant, non-tacky deposits that are resistant to at least two shampoo washes and to water, for application to the hair.

### Two-Gesture Application:

Base coat A: deposit of a compound 1 to 9 bearing acetoacetate functions, alone or diluted in volatile solvents such as ethanol, water, isododecane or a mixture of isododecane/ethanol or ethanol/water. Optionally, a crosslinking agent may be introduced into this phase to perform pre-crosslinking.
OR
Base coat B: deposit of a crosslinking agent 1 to 9, and then
Top coat A: deposit with a crosslinking agent 1 to 9 OR
Top coat B: deposit of a compound 1 to 9 bearing acetoacetate functions, alone or diluted in volatile solvents such as ethanol, water, isododecane or a mixture of isododecane/ethanol or ethanol/water.

### Example 1: Compound 1 with Amino Crosslinking Agent 1 (Acid Catalysis)

Compositions A and B as described below were prepared from mixtures of different solutions.

Solution 1, a 3-aminopropyltriethoxysilane (APTES) solution, is prepared according to the process below: pure APTES (APTES Silsoft A-1100 sold by the company Momentive Performance Materials) is mixed with water brought to pH=1 by addition of HCl, and ethanol is then added. Said mixture is then stirred using a VWR magnetic stirrer (rotation speed 500 rpm) for 24 hours at room temperature.

Solution 2, an aqueous-alcoholic solution comprising a pigment and the polyol acetoacetate, is prepared according to the process below: the pigment (iron oxide sold by the company Sun Chemical under the name SunPuro Red Iron Oxide) is dispersed in ethanol. The polyol acetoacetate compound sold by the company Kings Industries under the name K-Flex 7301 is then added to the pigment solution.

Composition A according to the invention was prepared by mixing solutions 1 and 2. Composition B is a polyol-free comparative composition.

The various compounds are present in the amounts below (expressed in g/100 g).

TABLE 1

| | | Composition | Composition |
|---|---|---|---|
| | | A | B |
| Compositions | | (Invention) | (comparative) |
| Solution 1 | 3-Aminopropyltriethoxysilane | 19.14 | 19.14 |
| | (APTES) | | |
| | Ethanol | 21.17 | 21.17 |
| | Water (pH = 1) | 4.67 | 4.67 |
| Solution 2 | Pigment (iron oxide sold | 2.39 | 2.39 |
| | by the company Sun | | |
| | Chemical under the name | | |
| | SunPuro Red Iron Oxide) | | |
| | Polyol (sold by the | 4.78 | — |
| | company Kings Industries | | |
| | under the name K-Flex | | |
| | 7301) | | |
| | Ethanol | qs | qs |

Each of the compositions A and B is applied to locks of dry natural hair containing 90% white hair strands, in a proportion of 1 g of composition per gram of lock. After applying compositions A and B to the locks of hair, the locks are combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the shampoo washing protocol described below.

Shampoo washing protocol: The locks of dyed hair are combed, moistened with water at 35° C. and then passed between the fingers five times for 5 seconds. The locks of hair are then squeezed dry between two fingers.

A standard shampoo (Garnier Ultra Doux) is applied uniformly to the dyed locks, in a proportion of 0.4 g of standard shampoo per gram of locks, the locks of hair being massaged gently along the length (6 passes) for 15 seconds, from the root to the end.

The locks of hair are then placed on a watch glass and left to stand for 1 minute.

Next, the locks of hair are rinsed with water while passing the locks between the fingers (15 passes). The locks of hair are then squeezed dry between two fingers before the next shampoo wash.

Once the tests of several shampoo washes have been performed, the locks of hair are combed and dried with a hairdryer.

Persistence protocol: The persistence of the colour of the locks was evaluated in the CIE L*a*b* system, using a Minolta Spectrophotometer CM3600A colorimeter (illuminant D65, angle 10°, specular component included).

In this L*a*b* system, L* represents the intensity of the colour, a* indicates the green/red colour axis and b* the blue/yellow colour axis.

The persistence of the colouring is evaluated by the colour difference ΔE between the dyed locks before shampooing, then after having undergone 1 and 3 or 5 shampoo washes according to the protocol described above. The lower the ΔE value, the more persistent the colour with respect to shampoo washing. The ΔE value is calculated according to the following equation:

$$Δ E = \sqrt{{( {L}^{⋆} - {L}_{o}^{⋆} )}^{2} + {( {a}^{⋆} - {a}_{o}^{⋆} )}^{2} + {( {b}^{⋆} - {b}_{o}^{⋆} )}^{2}}$$

In this equation, L*a*b* represent the values measured after dyeing the hair and after performing the shampoo washes, and L₀*a₀*b₀* represent the values measured after dyeing the hair but before shampoo washing.

The chromaticity is calculated according to the following equation:

$${C}^{*} = \sqrt{{a}^{* 2} + {b}^{* 2}}$$

The evaluation results are summarized in the table below:

### Colorimetric Measurements:

TABLE 2

| | Number of | | |
|---|---|---|---|
| | shampoo | | |
| | washes | C* | ΔE |
| Composition A | 0 | 27.38 | — |
| (invention) | 5 | 28.65 | 3.07 |
| Composition B | 0 | 35.26 | — |
| (comparative) | 5 | 19.39 | 26.13 |

The locks of hair dyed with composition A according to the invention have significantly lower ΔE values than the locks of hair dyed with comparative composition B. It is also seen that the chromaticity of the hair dyed with composition A according to the invention does not vary significantly, whereas the chromaticity of the hair dyed with comparative composition B decreases very significantly after five shampoo washes.

### Example 2: Compound 1 with an Amino Crosslinking Agent 1+PDMS-OH (Basic Catalysis)

Compositions C and D as described below were prepared from mixtures of different solutions.

Solution 3, a 3-aminopropyltriethoxysilane (APTES) solution, is prepared according to the process below: pure APTES (APTES Silsoft A-1100 sold by the company Momentive Performance Materials) is mixed with water brought to pH=1 by addition of HCl, and ethanol is then added. Said mixture is then stirred using a VWR magnetic stirrer (rotation speed 500 rpm) for 24 hours at room temperature.

Solution 4, which is an alcoholic solution of non-amino silicone comprising at least two reactive groups, is prepared according to the process below: the polydimethylsiloxane (PDMS) compound bearing hydroxyl end functions (481939 sold by the company Sigma-Aldrich) is diluted in ethanol in which the pigment (iron oxide sold by the company Sun Chemical under the name SunPuro Red Iron Oxide) is dispersed. The polyol acetoacetate compound sold by the company Kings Industries under the name K-Flex 7301 is then added to the pigment solution.

Composition C according to the invention was prepared by mixing solutions 3 and 4. Composition D is a comparative composition.

The various compounds are present in the amounts below (expressed in g/100 g).

TABLE 3

| | | Composition | Composition |
|---|---|---|---|
| | | C | D |
| Compositions | | (invention) | (comparative) |
| Solution 3 | 3-Aminopropyltriethoxysilane | 8.71 | 8.71 |
| | (APTES) | | |
| | Ethanol | 48.31 | 48.31 |
| | Water (pH = 1) | 1.06 | 1.06 |
| Solution 4 | PDMS-disilanol | 4.37 | 4.37 |
| | Pigment (iron oxide sold | 1.31 | 1.31 |
| | by the company Sun | | |
| | Chemical under the name | | |
| | SunPuro Red Iron Oxide) | | |
| | Polyol (sold by the | 1.74 | — |
| | company Kings Industries | | |
| | under the name K-Flex | | |
| | 7301) | | |
| | Ethanol | qs | qs |

Each of the compositions C and D is applied to locks of dry natural hair containing 90% white hair strands, in a proportion of 1 g of composition per gram of lock. After applying compositions C and D to the locks of hair, the locks are combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 4

| | Number of | | |
|---|---|---|---|
| | shampoo | | |
| | washes | C* | ΔE |
| Composition C | 0 | 32.21 | — |
| (invention) | 3 | 30.69 | 3.39 |
| | 5 | 30.09 | 5.32 |
| Composition D | 0 | 28.51 | — |
| (comparative) | 3 | 23.50 | 14.45 |
| | 5 | 17.89 | 22.44 |

The locks of hair dyed with composition A according to the invention have significantly lower ΔE values than the locks of hair dyed with composition B. It is also seen that the chromaticity of the hair dyed with composition C according to the invention does not vary significantly, whereas the chromaticity of the hair dyed with comparative composition D decreases very significantly after three shampoo washes or even five shampoo washes.

### Example 3: Compound 1 with Amino Crosslinking Agent 1+PDMS-OH (Acid Catalysis)

Compositions E and F as described below were prepared from mixtures of different solutions.

Solution 5, a 3-aminopropyltriethoxysilane (APTES) solution, is prepared according to the process below: pure APTES (APTES Silsoft A-1100 sold by the company Momentive Performance Materials) is mixed with water brought to pH=1 by addition of HCl, and ethanol is then added. Said mixture is then stirred using a VWR magnetic stirrer (rotation speed 500 rpm) for 24 hours at room temperature.

Solution 6, which is an alcoholic solution of non-amino silicone comprising at least two reactive groups, is prepared according to the process below: the polydimethylsiloxane (PDMS) compound bearing hydroxyl end functions (481939 sold by the company Sigma-Aldrich) is diluted in ethanol in which the pigment (iron oxide sold by the company Sun Chemical under the name SunPuro Red Iron Oxide) is dispersed. The polyol acetoacetate compound sold by the company Kings Industries under the name K-Flex 7301 is then added to the pigment solution.

Composition E according to the invention was prepared by mixing solutions 5 and 6. Composition F is a comparative composition.

The various compounds are present in the amounts below (expressed in g/100 g).

TABLE 5

| | | Composition | Composition |
|---|---|---|---|
| | | E | F |
| Compositions | | (invention) | (comparative) |
| Solution 5 | 3-Aminopropyltriethoxysilane | 14.43 | 14.43 |
| | (APTES) | | |
| | Ethanol | 12.77 | 12.77 |
| | Water (pH = 1) | 3.52 | 3.52 |
| Solution 6 | PDMS-disilanol | 7.22 | 7.22 |
| | Pigment (iron oxide sold by | 2.16 | 2.16 |
| | the company Sun Chemical | | |
| | under the name SunPuro | | |
| | Red Iron Oxide) | | |
| | Polyol (sold by the company | 2.89 | — |
| | Kings Industries under the | | |
| | name K-Flex 7301) | | |
| | Ethanol | qs | qs |

Each of the compositions E and F is applied to locks of dry natural hair containing 90% white hair strands, in a proportion of 1 g of composition per gram of lock. After applying compositions E and F to the locks of hair, the locks are combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 6

| | Number of | | |
|---|---|---|---|
| | shampoo | | |
| | washes | C* | ΔE |
| Composition E | 0 | 30.22 | — |
| (invention) | 5 | 28.28 | 3.00 |
| Composition F | 0 | 28.85 | — |
| (comparative) | 5 | 21.88 | 21.1 |

The locks of hair dyed with composition E according to the invention have lower ΔE values than the locks of hair dyed with composition F.

In addition, it is seen that the chromaticity of the hair coloured with composition E according to the invention does not vary significantly, whereas the chromaticity of the hair dyed with the comparative composition F decreases very significantly after five shampoo washes.

### Example 4: Compound 4 with Amino Crosslinking Agent 1 (without Solvent—One Gesture)

Composition G, as described below, was prepared from mixtures of different compounds.

### Composition G

TABLE 7

| Ingredients | % a.m.* |
|---|---|
| Compound 4 | 32.4 |
| Red iron oxide | 6 |
| APTES | 61.6 |
| Total | 100 |

*a.m.: active material

Composition G is applied to locks of dry natural hair containing 90% white hair strands, at a rate of 1 g of composition per gram of lock. After applying to the lock of hair, the lock is combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 8

| | Number of | | | |
|---|---|---|---|---|
| | shampoo | | | |
| | washes | L* | C* | ΔE |
| Composition G | 0 | 34.29 | 34.37 | — |
| | 5 | 34.93 | 32.88 | 1.49 |

The locks of hair dyed with composition G according to the invention do not show a significant reduction in colour even after five shampoo washes. It is thus seen that the process of the invention makes it possible to obtain colouring that is persistent with respect to shampoo washing. Furthermore, the colour intensity of composition G remains powerful after five shampoo washes.

### Example 5: Compound 5 with Amino Crosslinking Agent 1 (One Gesture)

Composition H, as described below, was prepared from mixtures of different compounds.

### Composition H

TABLE 9

| Ingredients | % a.m. |
|---|---|
| Compound 5 | 25 |
| Red iron oxide | 6 |
| APTES | 48 |
| Ethanol | 21 |
| Total | 100 |

Composition H is applied to locks of dry natural hair containing 90% white hair strands, at a rate of 1 g of composition per gram of lock. After applying to the lock of hair, the lock is combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 10

| | Number of | | | |
|---|---|---|---|---|
| | shampoo | | | |
| | washes | L* | C* | ΔE |
| Composition H | 0 | 34.71 | 30.67 | — |
| | 3 | 35.94 | 31.13 | 0.42 |
| | 5 | 36.04 | 29.41 | 1.76 |

The locks of hair dyed with composition H according to the invention do not show a significant reduction in colour even after three or even five shampoo washes. It is thus seen that the process of the invention makes it possible to obtain colouring that is persistent with respect to shampoo washing. Furthermore, the colour intensity of composition H remains powerful after three or even five shampoo washes.

### Example 6: Compound 5 Pre-Crosslinked with Amino Crosslinking Agent 2 Followed by a Second Gesture with Amino Crosslinking Agent 3

Compositions I and J as described below were prepared from mixtures of different compounds.

### Composition I

TABLE 11

| Ingredients | % a.m. |
|---|---|
| Compound 5 | 25 |
| Red iron oxide | 6 |
| Amino crosslinking agent 2 | 12 |
| Water/ethanol (50/50 v/v) | 57 |
| Total | 100 |

### Composition J (Second Gesture)

TABLE 12

| Ingredients | % a.m. |
|---|---|
| Amino crosslinking agent 3 | 5 |
| Isododecane | 95 |
| Total | 100 |

Composition I is applied to locks of dry natural hair containing 90% white hair strands, at a rate of 0.5 g of composition per gram of lock, and then combed. After this first gesture, composition J is applied to the lock of hair, at a rate of 0.5 g per gram of lock. After application, the locks are combed and dried with a hairdryer, and then left to rest for 24 hours in a fume hood at room temperature.

The hair is dyed uniformly and intensely. The locks of hair thus dyed are then subjected to a test of several repeated shampoo washes so as to evaluate the fastness (persistence) of the colouring obtained with respect to shampoo washing, according to the same protocol as that applied in Example 1.

The locks of hair dyed with the composition comprising Example 6 according to the invention have an intensity which remains powerful: L*=32.65 at 0 shampoo washes and L*=34.21 after three shampoo washes.

### Example 7: Compound 5 Pre-Crosslinked with Amino Crosslinking Agent 2 Followed by a Second Gesture with Thiol-Based Crosslinking Agent 5

Compositions K and L as described below were prepared from mixtures of different compounds.

### Composition K

TABLE 13

| Ingredients | % a.m. |
|---|---|
| Compound 5 | 25 |
| Red iron oxide | 6 |
| Amino crosslinking agent 2 | 12 |
| Water/ethanol (50/50 v/v) | 57 |
| Total | 100 |

### Composition L (Second Gesture)

TABLE 14

| Ingredients | % a.m. |
|---|---|
| Thiol-based crosslinking agent 5 | 5 |
| Isododecane | 95 |
| Total | 100 |

Composition K is applied to locks of dry natural hair containing 90% white hair strands, at a rate of 0.5 g of composition per gram of lock, and then combed. After this first gesture, composition L is applied to the lock of hair, at a rate of 0.5 g per gram of lock. After application, the locks are combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

After applying the composition of Example 7, it is seen that the colour obtained is powerful (L*=33.91) and chromatic (C*=25.36).

### Example 8: Styling Application: Styling Evaluation Protocol

The styling evaluation protocol is detailed below:

2 g of a solution containing the formulations described are sprayed onto the lock (90% Natural White (NW) hair, length 20.5 cm). The lock is weighed before and after application. About 0.5 g of the polymer-containing solution is effectively deposited on the lock.

A 1 g lock of keratin fibres (90% Natural White NW hair, length 20.5 cm) is wrapped around a brush (approximate diameter of 2 cm over a length of 3 cm). The lock is left at room temperature, at 25° C.

After 24 hours, the lock is released from the brush and suspended. The length of the lock is then measured. After 24 hours of suspension, the length of the lock is again measured.

A comparison is made with a lock on which only the reference formulations were sprayed.

Formulations 1, 2, 3, 5, 6, 8, 9, 11 and 12 are comparative “reference” formulations. Formulations 4, 7, 10 and 13 are the formulations according to the invention.

The compositions of the various formulations, as described below, were prepared from mixtures of different compounds.

### Formulation No. 1

TABLE 15

| Formulations | 1 | 2 | 3 | 4 | 5 | 6 | 7 |
|---|---|---|---|---|---|---|---|
| Water/ethanol 50/50 v/v | 100% | 90% | 90% | 89.5% | 90% | 90% | 90% |
| Compound 3 | — | 10% | — | 5.5% | — | — | — |
| Polyacrylate crosslinking agent 6 | — | — | 10% | 4.5% | — | — | — |
| 1,8-Diazabicyclo[5.4.0]undec-7-ene | — | — | — | 0.5% | — | — | — |
| Compound 2 | — | — | — | — | 10% | — | 6.2% |
| Amino crosslinking agent 4 | — | — | — | — | — | 10% | 3.8% |

TABLE 16

| Formulations | 8 | 9 | 10 | 11 | 12 | 13 |
|---|---|---|---|---|---|---|
| Water/ethanol | 90% | 90% | 90% | 90% | 90% | 90% |
| (50/50) | | | | | | |
| Compound 5 | 10% | | 5.1% | | | |
| Amino crosslinking | | 10% | 4.9% | | | |
| agent 2 | | | | | | |
| Compound 1 | | | | 10% | | 5% |
| Alkoxide | | | | | 10% | 5% |
| crosslinking agent 7 | | | | | | |

$$\begin{matrix}\% Boucle = \frac{( L 0 - Li )}{L 0} \\ L 0 = Length after application to a virgin lock \\ Li = Length after application to a lock i\end{matrix}$$

The evaluation results are summarized in the table below:

TABLE 17

| | Value after | |
|---|---|---|
| | 24 hours of | |
| | drying | |
| Formulations | (in cm) | % curl |
| Untreated lock | 20.5 | — |
| Formulation 1 | 8.5 | 58 |
| (comparative) | | |
| Formulation 2 | 7 | 65.8 |
| (comparative) | | |
| Formulation 3 | 11 | 46.3 |
| (comparative) | | |
| Formulation 4 | 5.5 | 73.1 |
| (invention) | | |
| Formulation 5 | 7 | 65.8 |
| (comparative) | | |
| Formulation 6 | 8.5 | 58.5 |
| (comparative) | | |
| Formulation 7 | 4.5 | 78 |
| (invention) | | |
| Formulation 8 | 7 | 65.8 |
| (comparative) | | |
| Formulation 9 | 8 | 61 |
| (comparative) | | |
| Formulation 10 | 3 | 85.3 |
| (invention) | | |
| Formulation 11 | 12 | 41.4 |
| (comparative) | | |
| Formulation 12 | 11.5 | 43.9 |
| (comparative) | | |
| Formulation 13 | 8 | 61 |
| (invention) | | |

TABLE 18

| | Value after | Value after | |
|---|---|---|---|
| | 24 hours of | 24 hours of | |
| | drying (in | suspension | Delta |
| Formulations | cm) | (in cm) | “relaxation” |
| Formulation 1 | 8.5 | 13 | 4.5 |
| (comparative) | | | |
| Formulation 5 | 7 | 10.5 | 3.5 |
| (comparative) | | | |
| Formulation 6 | 8.5 | 13 | 4.5 |
| (comparative) | | | |
| Formulation 7 | 4.5 | 6.5 | 2 |
| (invention) | | | |
| Formulation 8 | 7 | 11.5 | 4 |
| (comparative) | | | |
| Formulation 9 | 8 | 12 | 4 |
| (comparative) | | | |
| Formulation 10 | 3 | 3.5 | 0.5 |
| (invention) | | | |
| Formulation 11 | 12 | 15.5 | 3.5 |
| (comparative) | | | |
| Formulation 12 | 11.5 | 17 | 5.5 |
| (comparative) | | | |
| Formulation 13 | 8 | 11 | 3 |
| (invention) | | | |

It is seen that the locks treated with formulations 4, 7, 10 and 13 according to the invention significantly improve the shape of the curls, which appear sharper and closer together, unlike the comparative locks. In addition, even after 24 hours, the lock lost significantly less curl shape than the comparative locks.

### Example 9: Compound 7 Pre-Crosslinked with Amino Crosslinking Agent 1 Followed by a Second Gesture with Amino Crosslinking Agent 8

Compositions A1 and B1 as described below were prepared from mixtures of different compounds.

### Composition A1

TABLE 19

| Ingredients | % a.m. |
|---|---|
| Compound 7 | 10 |
| Red iron oxide | 6 |
| Amino crosslinking agent 1 | 4 |
| Water/isopropanol (70/30 v/v) | qs |

### Composition B1 (Second Gesture)

TABLE 20

| Ingredients | % a.m. |
|---|---|
| Amino crosslinking agent 8 | 5 |
| Water (2% lactic acid) | qs |

Composition A1 is applied to a lock of dry natural hair containing 90% white hair strands, at a rate of 0.5 g of composition per gram of lock, and then combed. After this first gesture, composition B1 is applied to the lock of hair, at a rate of 0.5 g per gram of lock.

After application, the lock is combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 21

| | Number of | | | | |
|---|---|---|---|---|---|
| | shampoo | | | | |
| | washes | L* | a* | b* | ΔE |
| Example 9 | 0 | 34.13 | 23.81 | 17.5 | — |
| | 3 | 33.98 | 23.33 | 17.57 | 0.57 |

The dyed lock shows good persistence of the colour after three shampoo washes.

### Example 10: Compound 8 Pre-Crosslinked with Amino Crosslinking Agent 9 Followed by a Second Gesture with Thiol-Based Crosslinking Agent 5

Compositions C1 and D1 as described below were prepared from mixtures of different compounds.

### Composition C1

TABLE 22

| Ingredients | % a.m. |
|---|---|
| Compound 8 | 10 |
| Yellow iron oxide | 6 |
| Amino crosslinking agent 9 | 7.45 |
| Water/isopropanol (70/30 v/v) | qs |

### Composition D1 (Second Gesture)

TABLE 23

| Ingredients | % a.m. |
|---|---|
| Thiol-based crosslinking agent 5 | 5 |
| Isododecane | qs |

Composition C1 is applied to a lock of dry natural hair containing 90% white hair strands, at a rate of 0.5 g of composition per gram of lock, and then combed. After this first gesture, composition D1 is applied to the lock of hair, at a rate of 0.5 g per gram of lock.

After application, the lock is combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 24

| | Number of | | | | |
|---|---|---|---|---|---|
| | shampoo | | | | |
| | washes | L* | a* | b* | ΔE |
| Example 10 | 0 | 47.55 | 13.44 | 38.29 | — |
| | 3 | 49.31 | 13.33 | 37.98 | 1.78 |

The dyed lock shows good persistence of the colour after three shampoo washes.

### Example 11: Compound 9 Pre-Crosslinked with Amino Crosslinking Agent 3 Followed by a Second Gesture with Thiol-Based Crosslinking Agent 5

Compositions E1 and F1 as described below were prepared from mixtures of different compounds.

### Composition E1

TABLE 25

| Ingredients | % a.m. |
|---|---|
| Compound 9 | 10 |
| Black iron oxide | 6 |
| Amino crosslinking agent 3 | 5.20 |
| Water/isopropanol (70/30 v/v) | qs |

### Composition F1 (Second Gesture)

TABLE 26

| Ingredients | % a.m. |
|---|---|
| Thiol-based crosslinking agent 5 | 5 |
| Isododecane | qs |

Composition E1 is applied to a lock of dry natural hair containing 90% white hair strands, at a rate of 0.5 g of composition per gram of lock, and then combed. After this first gesture, composition F1 is applied to the lock of hair, at a rate of 0.5 g per gram of lock.

After application, the lock is combed and dried with a hairdryer, and then left to rest for 24 hours in a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 27

| | Number of | | | | |
|---|---|---|---|---|---|
| | shampoo | | | | |
| | washes | L* | a* | b* | ΔE |
| Example 11 | 0 | 24.68 | 0.53 | 0.82 | — |
| | 3 | 28.41 | 0.52 | 1.97 | 3.90 |

The dyed lock shows good persistence of the colour after three shampoo washes.

### Example 12: Hair Dyeing with a Step of Applying a Steam Straightening Iron

Formulations G1, G1a, G1b, H1, H1a, H1b as described below, were prepared from mixtures of different compounds.

The contents are expressed as weight percentages of active material, relative to the total weight of the composition.

TABLE 28

| | Formulation | Formulation | Formulation | Formulation | Formulation | Formulation |
|---|---|---|---|---|---|---|
| Ingredients | G1 | G1a | G1b | H1 | H1a | H1b |
| Compound 9 | — | — | — | 20 | 20 | — |
| Compound 1 | 20 | 20 | — | — | — | — |
| Red iron oxide | 6 | 6 | 6 | — | — | — |
| Yellow iron oxide | — | — | — | 6 | 6 | 6 |
| Amino crosslinking agent 9 | 16 | — | 16 | — | — | — |
| Amino crosslinking agent 2 | — | — | — | 17.4 | — | 17.4 |
| Water/ethanol (50/50) | qs 100 | qs 100 | qs 100 | qs 100 | qs 100 | qs 100 |

Formulations G1, G1a, G1b, H1, H1a and H1b are applied to locks of dry natural hair containing 90% white hair strands, at a rate of 1 g of composition per gram of lock.

The locks of hair are then combed and dried with a hairdryer for 1 minute and 30 seconds, and then a steam straightener (Rowenta Steampod) is applied to said locks of hair with five passes at a temperature of 210° C.

The locks of hair are then subjected to a test of several repeated shampoo washes so as to evaluate the fastness (persistence) of the colouring obtained with respect to shampoo washes, according to the same protocol as that applied in Example 1.

The evaluation results are summarized in the table below:

TABLE 29

| | Number of | | | | |
|---|---|---|---|---|---|
| | shampoo | | | | |
| Formulations | washes | L* | a* | b* | ΔE |
| Formulation G1 | 0 | 30.31 | 22.94 | 16.28 | — |
| (invention) | 3 | 34.17 | 22.08 | 14.86 | 4.2 |
| Formulation G1a | 0 | 32.52 | 23 | 15.82 | — |
| (comparative) | 1 | 51.88 | 12.53 | 20.06 | 22.17 |
| Formulation G1b | 0 | 34.22 | 25.84 | 19.02 | — |
| (comparative) | 1 | 56.31 | 7.73 | 15.06 | 28.83 |
| Formulation H1 | 0 | 50.55 | 14.59 | 41.63 | — |
| (invention) | 5 | 51.42 | 16.21 | 39.63 | 2.71 |
| Formulation H1a | 0 | 50.39 | 13.1 | 35.23 | — |
| (comparative) | 1 | 51.68 | 3.41 | 13.89 | 23.64 |
| Formulation H1b | 0 | 49.93 | 13.87 | 36.68 | — |
| (comparative) | 3 | 61.14 | 3.58 | 18.26 | 23.89 |

The locks of hair dyed with compositions G1 and H1 according to the process of the invention have lower ΔE values than the locks of hair dyed with the comparative compositions G1a-G1b and H1a-H1b.

Thus, the process according to the invention affords a homogeneous coloured coating on the hair which shows improved resistance to shampoo washing.

### Example 13: Hair Dyeing

Compositions 11 to Q1 as described below were prepared from mixtures of different compounds.

The contents are expressed as weight percentages of active material, relative to the total weight of the composition.

TABLE 30

| Ingredients | I1 | J1 | K1 | L1 | M1 |
|---|---|---|---|---|---|
| Compound 9 | | | 9.8 | 5 | 9.8 |
| Compound 8 | | 9.8 | | | |
| Compound 7 | 9.8 | | | | |
| Chitosan oligosaccharide | 14.7 | 15.7 | 14.5 | | |
| (GU3511) sold by the | | | | | |
| company Glentham Life | | | | | |
| Sciences | | | | | |
| pH 9 | | | | | |
| Priamine 1075-LQ-(GD) | | | | 22.7 | |
| sold by the company Croda | | | | | |
| Polylysine (PL-25) sold | | | | | 10 |
| by the company JNC | | | | | |
| Red iron oxide | 6 | 6 | 6 | 6 | 6 |
| Water | | | | | 37 |
| Water/ethanol (50/50) | qs | qs | qs | qs | qs |
| | 100 | 100 | 100 | 100 | 100 |

TABLE 31

| Ingredients | N1 | O1 | P1 | Q1 |
|---|---|---|---|---|
| Compound 9 | | | 9.8 | 9.8 |
| Compound 8 | 9.8 | 9.8 | | |
| Compound 7 | | | | |
| Chitosan sold by the company Chibio | | | 14.2 | |
| Biotech - Aspergillus niger Chitosan | | | | |
| Oligosaccharide pH 9 | | | | |
| Chitosan oligosaccharide (GU3511) | | 15.4 | | |
| sold by the company Glentham Life | | | | |
| Sciences | | | | |
| pH 9 | | | | |
| (3-Aminopropyl)triethoxysilane | | 1.5 | | |
| (APTES) (CAS number: 919-30-2, | | | | |
| sold by Sigma-Aldrich) | | | | |
| Jeffamine T403 | | | | 11.3 |
| Polyetheramine (Mw = 440 g/mol) | | | | |
| AHEW (81 g/eq) | | | | |
| Amodimethicone | 10 | | | |
| Emulsion | | | | |
| Sold under the name Belsil ADM | | | | |
| LOG 1 by the company Wacker | | | | |
| Red iron oxide | 6 | 6 | 6 | |
| Yellow iron oxide | | | | 1.4 |
| Pearlescent Pigment Prestige Soft | | | | 4.6 |
| Bronze sold by the company Eckart | | | | |
| (CI 77491) | | | | |
| Water | 37 | | | |
| Water/ethanol (50/50) | qs | qs | qs | qs |
| | 100 | 100 | 100 | 100 |

### One-Gesture Application:

Compositions I1 to L1 and P1 to Q1 are applied to locks of dry natural hair containing 90% white hair strands, at a rate of 1 g of composition per gram of lock, then combed and dried with a hair dryer, and then left to rest for 24 hours under a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

### Two-Gesture Application:

A first composition, solution 1 (APTES at 10% in water at pH 10), is applied to locks of dry natural hair containing 90% white hair strands, in a proportion of 0.6 g of composition per gram of lock.

The locks of hair are combed and then left for 5 minutes. The locks are then lightly rinsed with water and then dried with a hairdryer for 1 minute and 30 seconds.

After this first gesture, a second composition M1, N1 or O1 is applied to the lock of hair, at a rate of 1 g of composition per gram of lock, and the locks are then combed and dried with a hairdryer, then left to rest for 24 hours under a fume hood at room temperature.

The hair is dyed uniformly and intensely.

The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 32

| | Number of | | | | |
|---|---|---|---|---|---|
| | shampoo | | | | |
| Compositions | washes | L* | a* | b* | ΔE |
| I1 | 0 | 28.18 | 24.99 | 16.65 | — |
| (one gesture) | 15 | 47.74 | 17.49 | 16.23 | 20.95 |
| J1 | 0 | 29.6 | 26.03 | 17.45 | — |
| (one gesture) | 15 | 44.38 | 19.63 | 17.05 | 16.11 |
| K1 | 0 | 28.36 | 25.26 | 16.95 | — |
| (one gesture) | 15 | 47.31 | 17.62 | 15.89 | 20.45 |
| L1 | 0 | 31.6 | 25.5 | 28.26 | — |
| (one gesture) | 15 | 38.39 | 19.3 | 14.7 | 16.38 |
| M1 | 0 | 29.49 | 25.65 | 17.78 | — |
| (two gestures) | 15 | 41.37 | 17.7 | 13.43 | 14.94 |
| N1 | 0 | 32.96 | 25.4 | 17.89 | — |
| (two gestures) | 15 | 52.71 | 13.14 | 15.05 | 23.41 |
| O1 (two gestures) | 0 | 29.36 | 25.87 | 17.77 | — |
| | 15 | 42.2 | 22.5 | 18.31 | 13.25 |
| P1 | 0 | 29.68 | 26.26 | 17.78 | — |
| (one gesture) | 15 | 45 | 20.7 | 18.42 | 16.31 |
| Q1 | 0 | 44.12 | 16.76 | 25.21 | — |
| (one gesture) | 15 | 45.98 | 17.48 | 26.27 | 2.25 |

The locks of hair dyed with compositions 11 to 01 show good persistence of the colouring after 15 shampoos washes.

### Example 14

### One-Gesture Application:

Compositions A2 and B2 as described below were prepared from mixtures of different compounds.

The contents are expressed as weight percentages of active material, relative to the total weight of the composition.

TABLE 33

| | A2 | B2 |
|---|---|---|
| Ingredients | (Invention) | (Comparative) |
| Compound 9 | 9.8 | — |
| Aqueous dispersion of | — | 9.8 |
| polyvinylpyrrolidone/isobornyl | | |
| acrylate/2-ethylhexyl | | |
| acrylate/acetoacetoxyethyl | | |
| methacrylate (2/59/29/10) | | |
| (aqueous dispersion 2 of | | |
| Example B of WO | | |
| 2022/136114) | | |
| Red iron oxide | 6 | 6 |
| Jeffamine T403 | 11.3 | 11.3 |
| Polyetheramine (Mw = 440 | | |
| g/mol) | | |
| AHEW (81 g/eq) | | |
| Water/ethanol (50/50) | qs 100 | qs 100 |

Composition A2 ad B2 are applied to locks of dry natural hair containing 90% white hair strands, at a rate of 1 g of composition per gram of lock, and then combed. After application, the locks are combed and dried with a hairdryer, and then left to rest for 24 hours in a fume hood at room temperature.

The hair is dyed uniformly and intensely. The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

TABLE 34

| | Number of | | | | |
|---|---|---|---|---|---|
| | shampoo | | | | |
| Compositions | washes | L* | a* | b* | ΔE |
| Composition A2 (invention) | 0 | 31.75 | 22.54 | 14.64 | — |
| | 3 | 31.91 | 21.97 | 15.59 | 1.11 |
| Composition B2 (comparative) | 0 | 33.83 | 27.01 | 20.22 | — |
| | 3 | 57.05 | 3.34 | 12.85 | 33.97 |

The locks of hair dyed with composition A2 according to the invention have a lower ΔE value than the locks of hair dyed with comparative composition B2.

Thus, the process according to the invention affords a coloured coating which shows improved resistance to shampoo washing, relative to the process using the comparative composition.

### Two-Gesture Application:

The following compositions A3, B3 and 03 as described below were prepared from mixtures of different compounds.

The contents are expressed as weight percentages of active material, relative to the total weight of the composition.

### Compositions A3 and B3

TABLE 35

| | A3 | B3 |
|---|---|---|
| Ingredients | (Invention) | (Comparative) |
| Compound 7 | 10 | — |
| Aqueous dispersion of | — | 10 |
| polyvinylpyrrolidone/acetoacetoxyethyl | | |
| methacrylate (10/90) | | |
| (aqueous dispersion 5 of Example E | | |
| of WO 2022/136114) | | |
| Red iron oxide | 6 | 6 |
| Amino crosslinking agent 1 | 4 | 4 |
| Water/isopropanol (70/30 v/v) | qs 100 | qs 100 |

### Composition C3 (Second Gesture)

TABLE 36

| Ingredients | C3 |
|---|---|
| Amino crosslinking agent 8 | 5 |
| Water (2% lactic acid) | qs 100 |

Composition A3 or B3 is applied to locks of dry natural hair containing 90% white hair strands, at a rate of 0.5 g of composition per gram of lock, and then combed. After this first gesture, composition C3 is applied to the locks of hair, at a rate of 0.5 g per gram of lock. After application, the locks are combed and dried with a hairdryer, and then left to rest for 24 hours in a fume hood at room temperature.

The hair is dyed uniformly and intensely. The locks of hair thus dyed are then subjected to a test of several repeated shampoo washes so as to evaluate the fastness (persistence) of the colouring obtained with respect to shampoo washing, according to the same protocol as that applied in Example 1.

The evaluation results are summarized in the table below:

### Colorimetric Measurements:

| | Number of | | | | |
|---|---|---|---|---|---|
| | shampoo | | | | |
| Compositions | washes | L* | a* | b* | ΔE |
| Composition A3 and then C3 | 0 | 34.13 | 23.81 | 17.5 | — |
| (Invention) | 3 | 33.98 | 23.33 | 17.57 | 0.51 |
| Composition B3 and then C3 | 0 | 34.5 | 21.78 | 14.98 | — |
| (Comparative) | 3 | 56.58 | 6.89 | 13.52 | 26.67 |

The locks of hair dyed with composition A3 and then composition C3 according to the process of the invention have a lower ΔE value than the locks of hair dyed with comparative composition B3 and then composition C3.

Thus, the process according to the invention affords a coloured coating which shows improved resistance to shampoo washing, relative to the process using the comparative composition.

1. A process for treating keratin fibres in one or more steps by applying to said fibres:
i) one or more compounds of formula (I), the optical isomers thereof, the geometrical isomers thereof, the salts thereof, the solvates thereof, the hydrates: 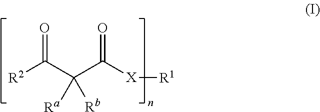
in which formula (I):
R¹ represents a linear or branched, saturated or unsaturated, conjugated or non-conjugated, acyclic or cyclic, aromatic or non-aromatic C₂ to C₁₄ polyvalent hydrocarbon-based radical,
R¹ also being:
a) optionally substituted with one or more hydroxyl OH or dialkylamino —N(R)₂ groups with R representing a (C₁-C₄)alkyl group, carboxyl —C(O)—OH, a vinyl group, and
b) optionally interrupted with one or more heteroatoms or groups chosen from O, S, carbonyl —C(O)—, amine —N(R′), or combinations thereof, in which R′ represents a hydrogen atom, a linear or branched alkyl group containing from 1 to 4 carbon atoms optionally substituted with at least one hydroxyl group (OH), or optionally substituted with —X—C(O)—C(Rₐ)(R_{b})—C(O)—R²;
R² represents a linear or branched, saturated or unsaturated C₁-C₆ monovalent hydrocarbon-based radical;
R^{a} and R^{b}, which may be identical or different, represent a hydrogen atom or a (C₁-C₄)alkyl group;
X is a heteroatom or group chosen from O, S, N(R′) or —S—CH₂—C(O)—O—, —O—C(O)—CH₂—S—, —O—C(O)—N(R′)—, —N(R′)—C(O)—O— or —N(R′)—C(O)—N(R′)—;
n denotes an integer ranging from 2 to 10, particularly ranging from 3 to 8; and
ii) at least one crosslinking agent; and
iii) optionally at least one cosmetic active agent.

2. The process according to claim 1, in which the compound(s) of formula (I) are chosen from those of formula (Ia), and also the optical isomers thereof, the geometrical isomers thereof, the salts thereof, and the solvates thereof, such as the hydrates: 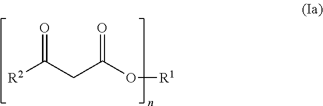
in which formula (Ta):
wherein R¹ represents a linear or branched, saturated or unsaturated, conjugated or non-conjugated, acyclic or cyclic, aromatic or non-aromatic C₂ to C₁₄, R¹ also being:
a) optionally substituted with one or more hydroxyl groups OH, and
b) optionally interrupted with one or more heteroatoms or groups chosen from O, S, —N(R′);
R² wherein R² represents a linear or branched (C₁-C₆)alkyl group; and
n being wherein n denotes an integer ranging from 2 to 8.

3. The process according to claim 1, in which the compound(s) of formula (I) or (Ta) are such that:
R¹ represents a linear or branched, unsaturated monocyclic or bicyclic C₆ to C₈ polyvalent hydrocarbon-based radical, or
R¹ represents a saturated monocyclic or bicyclic C₅ to C₁₂ polyvalent hydrocarbon-based radical, optionally interrupted with one or more heteroatoms or groups chosen from O, S, —N(R′), in which R′ represents a hydrogen atom or an alkyl group containing from 1 to 4 carbon atoms, or
R¹ represents a linear or branched, saturated or unsaturated, acyclic C₂ to C₁₂ polyvalent hydrocarbon-based radical.

4. The process according to claim 1, in which the compound(s) of formula (I) or (Ia) are such that:
n denotes an integer ranging from 2 to 8, and more particularly n denotes an integer ranging from 3 to 6, more particularly from 3 to 5.

5. The process according to claim 1, in which the compound(s) of formula (I) or (Ia) are chosen from the following compounds, and also the optical isomers thereof, the salts thereof, and the solvates thereof, such as hydrates:

| (Compound) | | |
|---|---|---|
| CAS No. | Chemical name | Chemical structure |
| (1) 700865-73-2 | butanoic acid 3-oxo- 1,1′-(1,7-heptanediyl) ester | 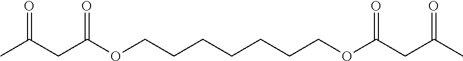 [SMILES: CC(=O)CC(=O)OCCCCCCCOC(=O)CC(C)=O] |
| (2) 632336-20-0 | butanoic acid 3-oxo- 1,1′-(1,8-octanediyl) ester | 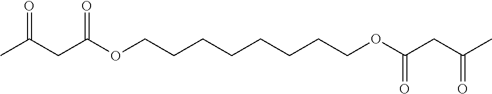 [SMILES: CC(=O)CC(=O)OCCCCCCCCOC(=O)CC(C)=O] |
| (3) 501426-98-8 | butanoic acid 3-oxo- 1,1′-(1,10-decanediyl) ester | 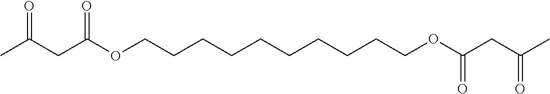 [SMILES: CC(=O)CC(=O)OCCCCCCCCCCOC(=O)CC(C)=O] |
| (4) 174498-06-7 | butanoic acid 3-oxo- 1,1′-(1,3-propanediyl) ester | 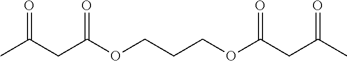 [SMILES: CC(=O)CC(=O)OCCCOC(=O)CC(C)=O] |
| (5) 160187-52-0 | butanoic acid 3-oxo- 1,2-dimethyl-1,2- ethanediyl ester | 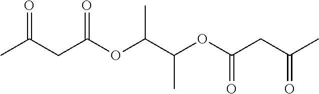 [SMILES: CC(=O)CC(=O)OC(C)C(C)OC(=O)CC(C)=O] |
| (6) 145020-19-5 | butanoic acid 3-oxo- 2-butyl-2-ethyl-1,3- propanediyl ester | 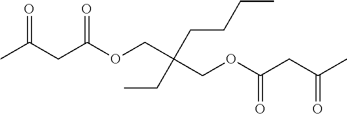 [SMILES: CCCCC(CC)(COC(=O)CC(C)=O)COC(=O)CC(C)=O] |
| (7) 87530-36-7 | butanoic acid 3-oxo- 1,1,2,2-tetramethyl- 1,2-ethanediyl ester | 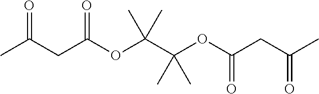 [SMILES: CC(=O)CC(=O)OC(C)(C)C(C)(C)OC(=O)CC(C)=O] |
| (8) 58213-75-5 | butanoic acid 3-oxo- 1,1′-(1-methyl-1,3- propanediyl)ester | 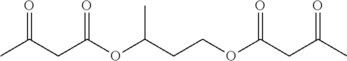 [SMILES: CC(=O)CC(=O)OCCC(C)OC(=O)CC(C)=O] |
| (9) 58213-74-4 | butanoic acid 3-oxo- 1,1′-[2,2-dimethyl-1- (1-methylethyl)-1,3- propanediyl]ester | 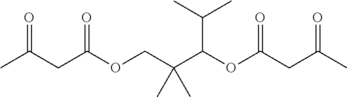 [SMILES: CC(=O)CC(=O)OCC(C)(C)C(OC(=O)CC(C)=O)C(C)C] |
| (10) 39564-28-8 | butanoic acid 3-oxo- 1,1′-(1,5-pentanediyl) ester | 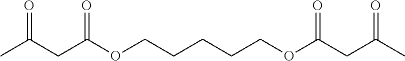 [SMILES: CC(=O)CC(=O)OCCCCCOC(=O)CC(C)=O] |
| (11) 14276-67-6 | butanoic acid 3-oxo- 1,1′-(2,2-dimethyl- 1,3-propanediyl) ester | 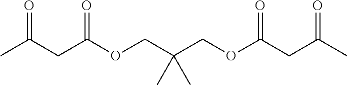 [SMILES: CC(=O)CC(=O)OCC(C)(C)COC(=O)CC(C)=O] |
| (12) 13018-41-2 | butanoic acid 3-oxo- 1,4-butanediyl ester | 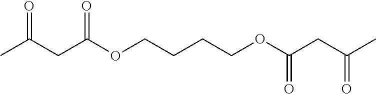 [SMILES: CC(=O)CC(=O)OCCCCOC(=O)CC(C)=O] |
| (13) 6079-90-9 | butanoic acid 3-oxo- 1-methyl-1,2- ethanediyl ester | 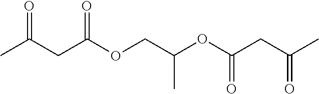 [SMILES: CC(=O)CC(=O)OCC(C)OC(=O)CC(C)=O] |
| (14) 5459-04-1 | butanoic acid 3-oxo- 1,2-ethanediyl ester | 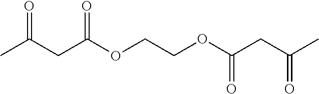 [SMILES: CC(=O)CC(=O)OCCOC(=O)CC(C)=O] |
| (15) 2388-18-3 | butanoic acid 3-oxo- 1,1′-(1,6-hexanediyl) ester butanoic acid 3-oxo- 1,6-hexanediyl ester (9CI) | 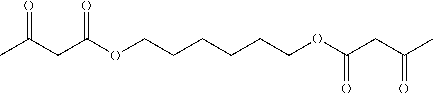 [SMILES: CC(=O)CC(=O)OCCCCCCOC(=O)CC(C)=O] |
| (16) 32818-60-3 | 1,1′-[2,2-bis[(1,3- dioxobutoxy)methyl]- 1,3-propanediyl] bis(3-oxobutanoate) | 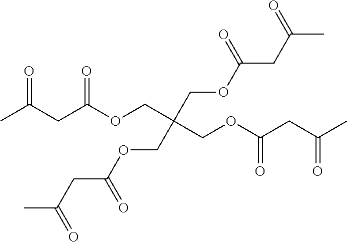 [SMILES: CC(=O)CC(=O)OCC(COC(=O)CC(C)=O)(COC(=O)CC(C)=O)COC(=O)CC(C)=O] |
| (17) 51980-20-2 | 1,1′-[2,2- bis(hydroxymethyl)- 1,3-propanediyl] bis(3-oxobutanoate) | 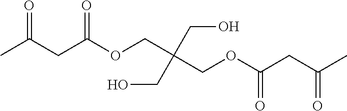 [SMILES: CC(=O)CC(=O)OCC(CO)(CO)COC(=O)CC(C)=O] |
| (18) | | 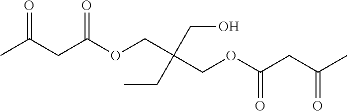 [SMILES: CCC(CO)(COC(=O)CC(C)=O)COC(=O)CC(C)=O] |
| (19) 130973-05-6 | butanoic acid 3-oxo- 1,3,5-benzenetriyl ester | 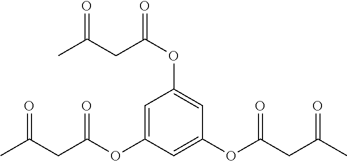 [SMILES: CC(=O)CC(=O)Oc1cc(OC(=O)CC(C)=O)cc(OC(=O)CC(C)=O)c1] |
| (20) 139264-86-1 | butanoic acid 3-oxo- 1,2,4-benzenetriyl ester | 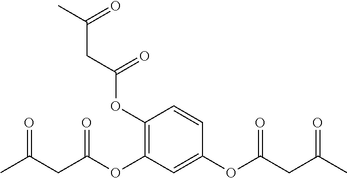 [SMILES: CC(=O)CC(=O)Oc1ccc(OC(=O)CC(C)=O)c(OC(=O)CC(C)=O)c1] |
| (21) 22208-25-9 | trimethylolpropane triacetoacetate K-FLEX 7301 | 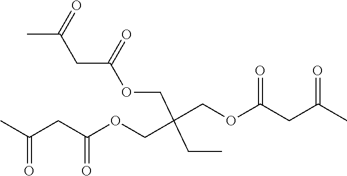 [SMILES: CCC(COC(=O)CC(C)=O)(COC(=O)CC(C)=O)COC(=O)CC(C)=O] |
| (22) 2360524-48- 5 | — | 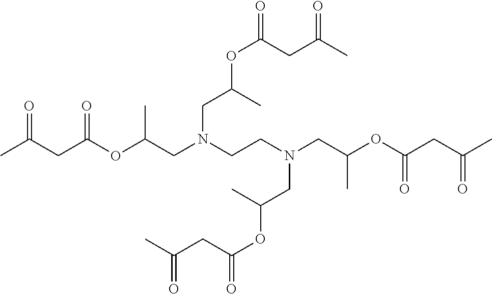 [SMILES: CC(=O)CC(=O)OC(C)CN(CCN(CC(C)OC(=O)CC(C)=O)CC(C)OC(=O)CC(C)=O)CC(C)OC(=O)CC(C)=O] |
| (23) 66229-33-2 | butanoic acid 3-oxo- 2-butyl-2-[(1,3- dioxobutoxy)methyl]- 1,3-propanediyl ester | 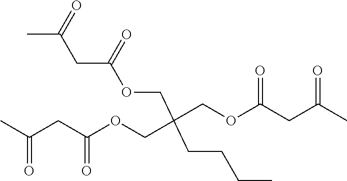 [SMILES: CCCCC(COC(=O)CC(C)=O)(COC(=O)CC(C)=O)COC(=O)CC(C)=O] |
| (24) 1579231-50- 7 | pentanoic acid 4,4- dimethyl-3-oxo-, 1,1′- [2,2-bis[[(4,4- dimethyl-1,3- dioxopentyl)oxy] methyl]-1,3- propanediyl]ester | 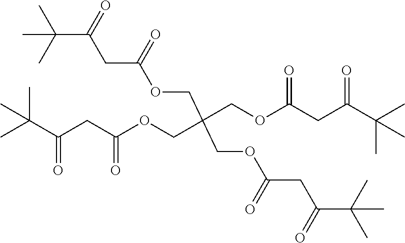 [SMILES: CC(C)(C)C(=O)CC(=O)OCC(COC(=O)CC(=O)C(C)(C)C)(COC(=O)CC(=O)C(C)(C)C)COC(=O)CC(=O)C(C)(C)C] |
| (25) 6079-98-7 | 1,1′,1″-(1,2,3- propanetriyl)tris(3- oxobutanoate) | 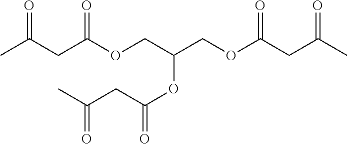 [SMILES: CC(=O)CC(=O)OCC(COC(=O)CC(C)=O)OC(=O)CC(C)=O] |
| (26) — | meso-erythrityl tetraacetoacetate | 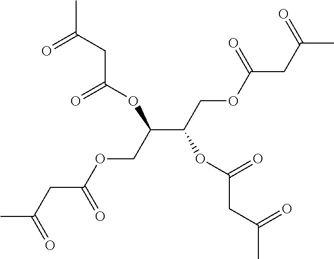 [SMILES: CC(=O)CC(=O)OC[C@H](OC(=O)CC(C)=O)[C@@H](COC(=O)CC(C)=O)OC(=O)CC(C)=O] |
| (27) 169533-29-3 | α-D-glucopyranose, 1,2,3,4,6-pentakis(3- oxobutanoate) | 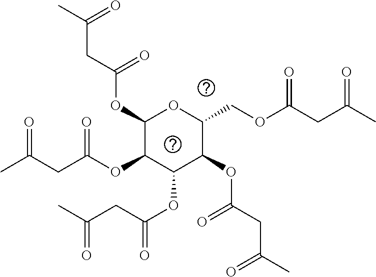 [SMILES: CC(=O)CC(=O)OC[C@H]1O[C@H](OC(=O)CC(C)=O)[C@H](OC(=O)CC(C)=O)[C@@H](OC(=O)CC(C)=O)[C@@H]1OC(=O)CC(C)=O] |
| (28) 2254710-55- 7 | benzoic acid 5-amino- 2-[[2,3,4-tris-O-(1,3- dioxobutyl)-D- xylopyranosyl]oxy]- | 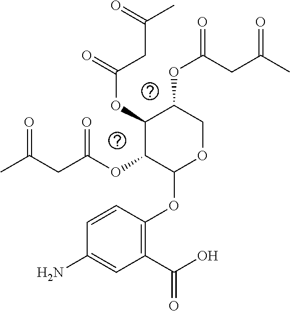 [SMILES: CC(=O)CC(=O)O[C@H]1[C@H](OC(=O)CC(C)=O)COC(Oc2ccc(N)cc2C(=O)O)[C@@H]1OC(=O)CC(C)=O] |
| (29) 1236300-10- 9 | 1,3,4,6-tetrakis-O- (1,3-dioxobutyl)-β-D- fructofuranosyl 2,3,4,6-tetrakis(3- oxobutanoate) α-D- glucopyranoside | 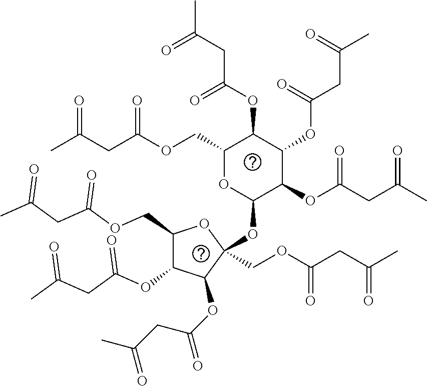 [SMILES: CC(=O)CC(=O)OC[C@H]1O[C@@](COC(=O)CC(C)=O)(O[C@H]2O[C@H](COC(=O)CC(C)=O)[C@@H](OC(=O)CC(C)=O)[C@H](OC(=O)CC(C)=O)[C@H]2OC(=O)CC(C)=O)[C@@H](OC(=O)CC(C)=O)[C@@H]1OC(=O)CC(C)=O] |
| (30) 96481-26-4 | D-glucopyranose pentakis(3- oxobutanoate) | 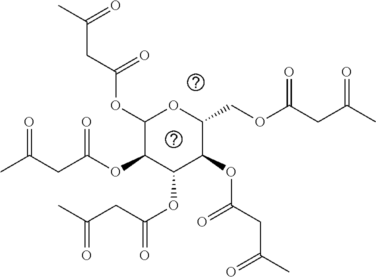 [SMILES: CC(=O)CC(=O)OC[C@H]1OC(OC(=O)CC(C)=O)[C@H](OC(=O)CC(C)=O)[C@@H](OC(=O)CC(C)=O)[C@@H]1OC(=O)CC(C)=O] |
| (31) 944473-12-5 | D-glucitol, 1,4:3,6- dianhydro-, 2,5-bis(3- oxobutanoate) | 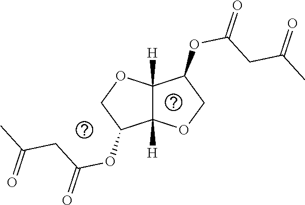 [SMILES: CC(=O)CC(=O)O[C@H]1CO[C@H]2[C@@H]1OC[C@H]2OC(=O)CC(C)=O] |
| (32) 2254710-49- 9 | — | 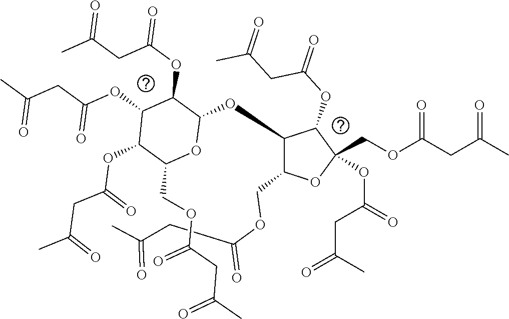 [SMILES: CC(=O)CC(=O)OC[C@H]1O[C@@H](O[C@@H]2[C@@H](COC(=O)CC(C)=O)O[C@@](COC(=O)CC(C)=O)(OC(=O)CC(C)=O)[C@H]2OC(=O)CC(C)=O)[C@H](OC(=O)CC(C)=O)[C@@H](OC(=O)CC(C)=O)[C@H]1OC(=O)CC(C)=O] |
| (33) — | sorbitol hexaacetoacetate | 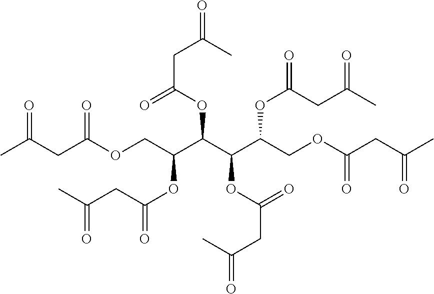 [SMILES: CC(=O)CC(=O)OC[C@H](OC(=O)CC(C)=O)[C@@H](OC(=O)CC(C)=O)[C@H](OC(=O)CC(C)=O)[C@@H](COC(=O)CC(C)=O)OC(=O)CC(C)=O] |
| (34) 216756-89-7 | D-glucitol, 4-O-β-D- galactopyranosyl-, polyacetoaceate | 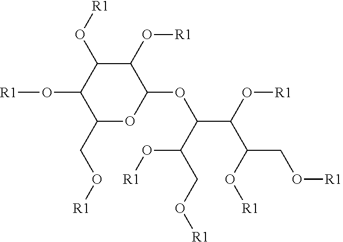 |
| | | with R1 chosen from —C(O)—CH₂— |
| | | C(O)—CH₃, H |
| | | with at least two radicals R1 |
| | | representing —C(O)—CH₂—C(O)—CH₃— |
| (35) 101655-52-1 | sucrose polyacetoacetate | 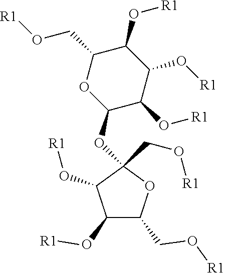 |
| | | with R1 chosen from —C(O)—CH₂—C(O)—CH₃, H |
| | | with at least two radicals R1 representing |
| | | —C(O)—CH₂—C(O)—CH₃ |
| (36) | glucose polyacetoacetate | 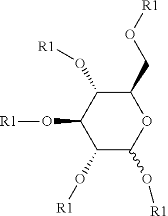 |
| (37) | mannose polyacetoacetate | 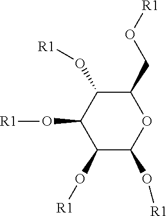 |
| (38) | xylitol polyacetoacetate | 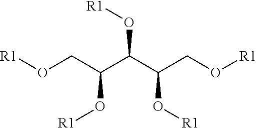 |
| (39) | sorbitol polyacetoacetate | 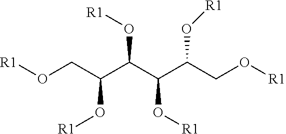 |
| | | six radicals R1 representing |
| | | —C(O)—CH₂—C(O)—CH₃ |
| (40) | | 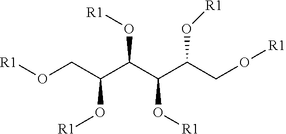 |
| | | three radicals R1 representing |
| | | —C(O)—CH₂—C(O)—CH₃, |
| | | and three radicals R1 representing a |
| | | hydrogen atom |
| (41) | 1471-94-9 | 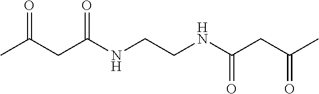 [SMILES: CC(=O)CC(=O)NCCNC(=O)CC(C)=O] |
| (42) | 2552-36-5 | 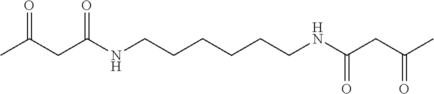 [SMILES: CC(=O)CC(=O)NCCCCCCNC(=O)CC(C)=O] |
| (43) | 139614-56-5 | 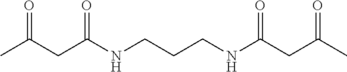 [SMILES: CC(=O)CC(=O)NCCCNC(=O)CC(C)=O] |
| (44) | 96169-30-1 | 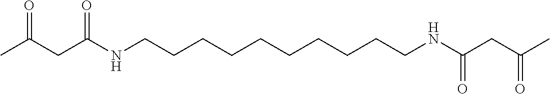 [SMILES: CC(=O)CC(=O)NCCCCCCCCCCNC(=O)CC(C)=O] |
| (45) | 75639-01-9 | 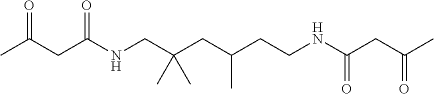 [SMILES: CC(=O)CC(=O)NCCC(C)CC(C)(C)CNC(=O)CC(C)=O] |
| (46) | dipentaerythritol polyacetoacetate | 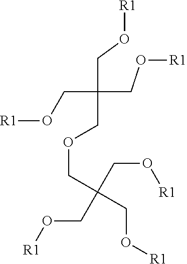 |
| | | five or six radicals R1 |

in which R1, which may be identical or different, represents a hydrogen atom or a —C(O)—CH₂—C(O)—CH₃ group, it being understood that at least two radicals R1 represent —C(O)—CH₂—C(O)—CH₃.

6. Process according to claim 1, in which the crosslinking agent(s) ii) are chosen from:
(poly)amine, (poly)thiol, (poly)carbonyl, (poly)acrylate, metal alkoxide compounds, or mixtures thereof.

7. The process according to claim 1, in which the crosslinking agent(s) are chosen from:
(poly)amine compounds chosen from a) chitosans, such as poly(D-glucosamine), b) polyether diamines, particularly polyethylene glycol α,ω-diamines (bearing an amine function at the end of the chain) and 4,7,10-trioxa-1,1-tridecanediamine, c) polyether triamines, such as polyetheramines (or Jeffamine), d) aminoalkoxysilanes, such as APTES, e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH2) and amodimethicones comprising amine groups on side chains, such as bis-cetearyl amodimethicone, f) polyamine alkylenes such as those of formula (XII′) H₂N-Alk-NH₂, Alk being a (C₁-C₆)alkylene group optionally substituted with one or more amino group(s) and optionally interrupted with one or more —N(H)— such as spermidine; and
(poly)thiol compounds chosen from a) polydialkylsiloxanes bearing thiol functions, and b) alkoxysilanes bearing thiol functions, and in particular are chosen from a) polydialkylsiloxanes bearing thiol functions comprising polydimethylsiloxanes comprising thiol groups on the side chain, notably those of formula (XIII) 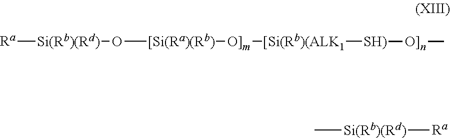
in which formula (XIII):
R^{a} and R^{b}, which may be identical or different, amd represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy; and R^{d} is as defined for R^{a} and R^{b}
R^{d} may also represent a (C₁-C₆)alkyl group substituted with a (C₁-C₄)alkylamino or amino or thiol group;
ALK₁ represents a linear or branched, optionally cyclic, saturated or unsaturated divalent hydrocarbon-based chain comprising from 1 to 100 carbon atoms, optionally interrupted with one or more heteroatoms selected oxygen, sulfur or nitrogen, a (thio)carbonyl group C(X) with X representing O or S, or combinations thereof;
n and m, which may be identical or different, representing an integer greater than 2 wherein the values of m and n are such that the weight-average molecular weight of said polyorganosiloxane is between 1000 and 55 000 g·mol⁻¹;
(poly)acrylate of formula (XIV) L[-Y—C(O)—C(R^{e})═CH₂]q (XIV)
in which formula (XIV):
q represents an integer greater than or equal to 2;
L denotes a saturated or unsaturated linear or branched, or a saturated or unsaturated (hetero)cyclic, multivalent (at least divalent) group comprising from 1 to 500 carbon and silicon atoms;
L being optionally interrupted or terminated with one or more heteroatoms or groups chosen from O, S, N, Si and C(X), or combinations thereof; and
L being optionally substituted with one or more groups chosen from: 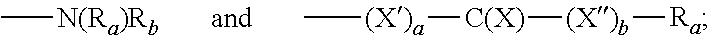
with X, X′ and X″, which may be identical or different, representing an oxygen or sulfur atom, or a group N(R_{b}); a and b being equal to 0 or 1;
Rₐ and R_{b}, which may be identical or different, represent a hydrogen atom or a (C₁-C₆)alkyl or aryl(C₁-C₄)alkyl group;
R^{e} represents a hydrogen atom or a (C₁-C₄)alkyl; and
Y representing an oxygen atom or an amino group —N(H)— L represents a di- or trivalent hydrocarbon-based chain comprising from 1 to 8 carbon atoms, q is 2 or 3;
metal alkoxides chosen from those of formula (XIVa) 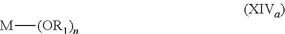
in which formula (XIVₐ):
M represents an atom chosen from alkaline-earth metals, transition metals, metals of the lanthanide family, post-transition metals such as aluminium or tin or metalloids such as boron;
n represents the valency of the atoms represented by M;
R1 represents a linear or branched, saturated or unsaturated hydrocarbon-based group containing from 1 to 30 carbon atoms, said hydrocarbon-based group being optionally interrupted with 1 to 20 heteroatoms chosen from O, N, S or P; and said hydrocarbon-based group being optionally substituted with one or more hydroxyl or carbonyl group,
wherein M represents an atom chosen from transition metals.

8. The process according to claim 1, which uses iii) one or more cosmetic active agent(s) chosen from a) dyestuffs such as pigments, direct dyes or mixtures thereof, b) active agents for caring for keratin fibres, c) UV-screening agents, or d) mixtures thereof.

9. The process according to claim 1, which uses iv) one or more fatty substances.

10. The process according to claim 1, which uses iv) one or more volatile oil(s) chosen from:
hydrocarbon-based oils containing from 8 to 16 carbon atoms selected from:
branched C₈-C₁₆ alkanes;
linear alkanes, for example C₁₁-C₁₆ alkanes, alone or as mixtures; or
volatile, non-aromatic cyclic C₅-C₁₂ alkanes;
short-chain esters containing from 3 to 8 carbon atoms in total
carbonate hydrocarbon-based oils of structure R′₁—O—C(O)—O—R′₂ in which R′₁ and R′₂ independently denote a linear, branched or cyclic C₄-C₈ alkyl group;
ether oils of formula R₁—O—R₂ in which R¹ and R₂ independently denote a linear, branched or cyclic C₄-C₈ alkyl group; or
silicone oils comprising from 2 to 7 silicon atoms, the silicone oils optionally including alkyl or alkoxy groups containing from 1 to 10 carbon atoms.

11. The process according to claim 1, in particular for caring for, styling or dyeing keratin fibres which uses:
a composition, termed “C1”, comprising i) at least one compound of formula (I) or (Ia) and optionally iii) at least one cosmetic active agent;
a composition, termed “C2”, comprising i) at least one compound of formula (I) or (Ia) and ii) at least one crosslinking agent and optionally iii) at least one active agent;
a composition, termed “C3”, comprising i) at least one compound of formula (I) or (Ia) ii) at least one crosslinking agent and iii) at least one cosmetic active agent;
a composition “C4” comprising ii) at least one crosslinking agent and optionally iii) at least one cosmetic active agent; or
a composition “C5” comprising iii) at least one cosmetic active agent,
wherein
the process uses, together or separately, i) at least one compound of formula (I) or (Ia) and ii) at least one crosslinking agent and,
wherein
compositions C1, C2, C3, C4 and C5 may be anhydrous, aqueous or aqueous-alcoholic and may comprise one or more fatty substances iv).

12. The process according to claim 11, which is performed in one gesture by applying composition C2 or C3 to keratin fibres.

13. The process according to claim 11, which is performed in two gestures by successive application, to keratin fibres, of at least two different compositions selected from C1 and C4, C2 and C4, C3 and C4, C1 then C4, C2 then C4, C3 then C4, or_C2 or C3 and then C4.

14. The process according to claim 11, which is a process for dyeing keratin fibres, in particular hair, comprising at least the sequential application:
of a composition C2 as defined in claim 11;
of a composition C4 as defined in claim 11; and
of a composition C5 as defined in claim 11;
wherein compositions C2 and/or C4 and/or C5 contain at least one dyestuff as defined in claim 8.

15. A method for treating keratin fibres with a combination of ingredients according to claim 1, comprising use of at least one ingredient i) combined with at least one ingredient ii) and optionally combined with at least one ingredient iii) and optionally combined with at least one ingredient iv) for caring for, styling, holding shape (curls) of or colouring keratin fibres.
